(19) United States
(12) Patent Application Publication
(10) Pub. No.: US 2024/0018594 A1
(43) Pub. Date: Jan. 18, 2024

(54) CIRCULATING miRNA AND PROTEIN BIOMARKERS FOR FACIOSCAPULOHUMERAL DYSTROPHY

(71) Applicant: CHILDREN'S NATIONAL MEDICAL CENTER, Washington, DC (US)

(72) Inventors: Yi-Wen CHEN, Washington, DC (US); Christopher HEIER, Washington, DC (US)

(73) Assignee: CHILDREN'S NATIONAL MEDICAL CENTER, Washington, DC (US)

(21) Appl. No.: 18/251,654

(22) PCT Filed: Nov. 3, 2021
(86) PCT No.: PCT/US2021/057881

§ 371 Date: May 3, 2023

Related U.S. Application Data
(60) Provisional application No. 63/109,561, filed on Nov. 4, 2020.

(51) Int. Cl. C12Q 1/6883; G01N 33/68; C12N 15/113; A61K 45/06

(52) U.S. Cl. CPC C12Q 1/6883; G01N 33/6893; C12N 15/113; A61K 45/06; C12Q 2600/158; C12Q 2600/178; G01N 2800/2878; G01N 2800/50

(57) ABSTRACT

A method for detecting or monitoring FSHD comprising detecting one or more biomarkers, such as miRNA biomarkers or protein biomarkers that are significantly decreased or increased in subjects having FSHD compared to normal control subjects. Methods for treatment of subjects at risk of having, or having FSHD.

### CROSS-REFERENCE TO RELATED APPLICATION

This application claims priority to U.S. Provisional No. 63/109,561, filed Nov. 4, 2020, which is hereby incorporated by reference for all purposes.

### BACKGROUND OF THE INVENTION

Field of the Invention. The invention pertains to the fields of medicine and specifically to markers and therapeutics for muscle diseases such as facioscapulohumeral dystrophy (“FSHD”).

Description of Related Art Facioscapulohumeral muscular dystrophy (FSHD) is an autosomal dominant muscle disorder with no accepted current therapy, a variable prognosis, and complex genetic and molecular mechanisms. FSHD is caused by aberrant expression of double homeobox 4 (DUX4) due to epigenetic changes of the D4Z4 repeat region at chromosome 4q35 [1-3].

Roughly 95% of patients have type 1 FSHD (“FSHD1”) due to contraction of the D4Z4 array; a small portion (˜5%) of patients have type 2 FSHD (“FSHD2”) caused by mutations in the structural maintenance of chromosomes flexible hinge domain containing 1 (SMCHD1) gene, the DNA methyltransferase 3B (DNMT3B) gene, or the ligand-dependent nuclear receptor-interacting factor 1 (LRIF1) gene [4-6].

The aberrant expression of DUX4 causes misregulation of genes involved in germline function, oxidative stress responses, myogenesis, post-transcriptional regulation, and additional cellular functions [7-13]. These downstream molecular changes are believed to cause FSHD, although the exact mechanisms are not clear.

While the onset of FSHD is generally around adolescent years, a small portion (˜4%) of patients present with an early-onset or infantile form of FSHD [14]. Previous studies have shown that the disease severity of FSHD is negatively correlated with the size of D4Z4 repeats [15, 16]. Individuals with early onset FSHD tend to have smaller D4Z4 repeats and more severe disease phenotypes, including more profound muscle weakness, younger age at loss of independent ambulation, and extramuscular manifestations such as retinal vasculopathy or hearing loss [14, 15, 17, 18].

In clinical practice, particularly with pediatric onset FSHD, there is a low utilization of serial histological assessments because patients require painful biopsies of muscle tissue which typically has patchy or uneven pathology. Given this, many patients opt to no longer undergo muscle biopsy once a genetic diagnosis is made. Functional motor scales provide a non-invasive alternative to study neuromuscular disease progression; however, they can show great variability, can be age- or disease stage-limited, and they can be subject to placebo or coaching effects in clinical trials [19, 20].

Circulating molecular biomarkers provide an exciting alternative to these clinical assessments because they provide objective measurements that can be assayed repeatedly over time using minimally invasive methods. Blood-based miRNAs or proteins that measure the progression of disease or a patient response to therapy over time are known as a monitoring biomarkers [21].

In clinical trials, monitoring biomarkers may also be used as pharmacodynamic biomarkers to identify patients who are early responders to therapy, to demonstrate exposure-response relationships, or to improve statistical power and modeling. However, as patient populations are sensitive and limited for this relatively rare pediatric disease, the development of less invasive monitoring or pharmacodynamic biomarkers will be important for detection and characterization of early-onset FSHD, as frequent serial biopsies are especially problematic in this population.

In view of the above limitations and drawbacks of present methods, the inventors sought to identify circulating biomarkers for FSHD and develop tests which could easily and non-invasively detect or monitor patients having or at risk of developing FSHD, improve clinical management of patients having FSHD, and which would facilitate the identification of new treatments for FSHD.

As disclosed herein, plasma samples from a cohort of individuals with early-onset FSHD1 were obtained and evaluated using both miRNA and proteomic profiling approaches with the objective of identifying molecules that can be used to monitor FSHD disease activity, and facilitate the development of therapeutics for FSHD. Initial analysis of a discovery group by the inventors identified a panel of miRNAs and proteins as correlating with FSHD. Bioinformatic analyses of ChIP-seq data provided a rationale for changes in these biomarkers as their behavior was found to be consistent with changes in transcription factor pathways that are disrupted in FSHD1. Subsequent characterization in separate, non-overlapping groups of mild FSHD1 patients validated nine biomarkers whose expression could be conveniently assayed by qRT-PCR or ELISA, and are increased in early-onset FSHD.

### BRIEF SUMMARY OF THE INVENTION

The foregoing paragraphs have been provided by way of general introduction, and are not intended to limit the scope of the following claims. The described embodiments, together with a description of further advantages, will be best understood by reference to the following detailed description taken in conjunction with the accompanying drawings.

One aspect of this technology is directed to detection, diagnosis, monitoring, or prognosis of facioscapulohumeral dystrophy (“FSHD”) using biomarkers that are easily, rapidly and minimally invasively obtainable from blood or other biofluid samples.

Another aspect of this technology is directed to methods for identifying therapeutic products and methods for prevention, reduction of the severity of, or treatment of FSHD by identifying or targeting abnormal levels of these biomarkers.

Embodiments of this technology include, but are not limited to the following.

A method for detecting or monitoring FSHD or DUX4 activities in a subject comprising, consisting essentially of, or consisting of: detecting at least one biomarker for FSHD present in a biofluid or liquid biopsy sample of the subject; comparing quantity of the at least one biomarker in the subject to a control value, preferably to the quantity in an age and gender matched subject who does not have FSHD, or to the quantity in a serially collected sample from the same subject at a different point in time; and optionally, treating the subject for FSHD when said biomarker is elevated or depressed compared to the control value. In one embodiment, this method will detect the miR-100 marker in plasma or serum. Other specific embodiments detect miR-29b, miR-34a, miR-505; and/or miR-576; and/or S100A8, F13A1, IGF1, PFN1, FBLN1, CFL1, TMSB4X, TPM4, EFEMP1, KRT16, SPP2, PROC, and/or PRG4.

In some embodiments, this method will further comprise selecting a particular subject for treatment based on significant differences in the quantity of one or more biomarkers, for example, when a biomarker is depressed or elevated compared to a control value. One or more subjects may be selected and separated from other subjects at lower risk for FSHD or not having FSHD. Selection may be done by a medical professional reviewing measurement of biomarkers disclosed herein as well as other clinical or medical data on a subject or automatically by algorithm or computer program provided with the results of tests for the biomarkers and other genetic, medical or clinical data. In some embodiments, a medical or surgical procedure or protocol will comprise measurement or detection of the FSHD biomarkers disclosed herein. In other embodiments, the detection of measurement of the FSHD biomarkers will occur independently of a medical or surgical procedure or protocol. Such independent uses include measurement of FSHD biomarkers in vitro or remote (away from physician or medical practitioner), office, mail-in or at-home biomarker testing.

Unless otherwise specified or limited, the phrase “detecting or monitoring FSHD” includes detecting or monitoring FSHD, detecting or monitoring an early disposition toward FSHD or risk of acquiring FSHD, monitoring development or progression of FSHD, monitoring a subject's response to prevention or treatment of FSHD, or use of prognostic biomarkers, such as those disclosed herein to provide a FSHD prognosis.

Another embodiment is directed to a method for detecting, diagnosing, monitoring or prognosing FSHD or a risk of FSHD in a subject comprising, consisting essentially of, or consisting of detecting at least one biomarker for FSHD present in blood, plasma or serum of a subject, comparing a quantity of said biomarker to a control value or to the quantity of the same biomarker in an age and gender matched subject who does not have FSHD; selecting a subject having, or at risk of having FSHD, when the quantity of said biomarker is significantly elevated or decreased compared to the control value; and, optionally, treating the subject for FSHD.

In some embodiments, the control value will be that of one or more subjects not having FSHD, at a low risk of developing FSHD based on other diagnostic or genetic criteria, or having mild or severe FSHD.

In some embodiments, the method is performed in order to diagnose the risk of or presence of FSHD. In other embodiments, the method is performed in order to establish the absence of, or a low risk of, FHSD, mild FSHD, or severe FSHD.

Qualitative or quantitative measurements of biomarkers may be obtained and used to assess the subject's status, preferably from blood, plasma or serum. In some alternative embodiments, the biomarker may be obtained from urine, sweat, tears, breast milk, bile, interstitial fluid, cytosol, peritoneal fluid, pleural fluid, amniotic fluid, semen, synovial (joint) fluid, CSF (cerebrospinal fluid), lymph, mucous, saliva, or other bodily fluids, stool or fecal matter, or epithelium, hair follicles, or mucosal cells or secretions (such as from bronchial, nasal, buccal, or cheek swabs), or biopsy, such as a muscle biopsy.

In some embodiments, the biomarker may be measured in a liquid biopsy, defined as sample obtained through a diagnostic procedure performed in order to detect or quantify biomarkers contained in the blood, plasma, serum, or other biofluids of a subject. Samples may be obtained from liquid biopsies such as those described by and incorporated by reference to Picher, Andy. Liquid Biopsy, Key for Precision Medicine. GENETIC ENGINEERING & BIOTECHNOLOGY NEWS, 2018).

This method may be practiced with a kit comprising reagents suitable for detecting or quantifying the specific miRNAs or proteins disclosed herein, such as complementary oligonucleotides, or with reagents suitable for detecting or quantifying the biomarker proteins disclosed herein, such as peptide specific antibodies.

A nucleic acid, complementary oligonucleotide to a miRNA, antibody to biomarker protein or pharmaceutical composition may be provided in any suitable form, e.g. in solid, lyophilized, liquid or substrate-bound form.

A kit or kit-of-parts may be a kit of two or more parts and typically comprises its components in suitable containers. For example, each container may be in the form of vials, bottles, squeeze bottles, jars, sealed sleeves, envelopes or pouches, tubes or blister packages or any other suitable form provided the container is configured so as to prevent premature mixing of components. Each of the different components may be provided separately, or some of the different components may be provided together (i.e. in the same container).

A container may also be a compartment or a chamber within a vial, a tube, a jar, or an envelope, or a sleeve, or a blister package or a bottle, provided that the contents of one compartment are not able to associate physically with the contents of another compartment prior to their deliberate mixing by a pharmacist or physician.

A kit may contain two, three, four or more containers, packs, or dispensers together with instructions for preparation of a sample for detection of miRNA or an FSHD biomarker protein.

In some embodiments, the kit comprises at least one container comprising the oligonucleotides complementary to the miRNAs disclosed herein, such as the nine validated miRNA biomarkers disclosed herein, and/or one or more antibodies that recognize the biomarker proteins, such as F13A1, IGF1, S100A8, PFN1, FBLN1, CFL1, TMSB4X, TPM4, EFEMP1, KRT16, SPP2, PROC, and/or PRG4 described herein.

The kit may contain a second container comprising a means for isolation, purification, maintenance, use, and/or storage of a sample comprising the miRNAs or biomarker proteins such as storage buffer.

A kit for detection of biomarker proteins may comprise a containing secondary antibodies or chemical indicators for antibody binding which may be lyophilized or in solution. The compositions included in the kit may be supplied in containers of any sort such that the shelf-life of the different components are preserved, and are not adsorbed or altered by the materials of the container. For example, suitable containers include simple bottles that may be fabricated from glass, organic polymers, such as polycarbonate, polystyrene, polypropylene, polyethylene, ceramic, metal or any other material typically employed to hold reagents or food; envelopes, that may consist of foil-lined interiors, such as aluminum or an alloy. Other containers include test tubes, vials, flasks, and syringes. The containers may have two compartments that are separated by a readily removable membrane that upon removal permits the components of the compositions to mix. Removable membranes may be glass, plastic, rubber, or other inert material.

Kits may also be supplied with instructional materials. Instructions may be printed on paper or other substrates, and/or may be supplied as an electronic-readable medium, such as a floppy disc, CD-ROM, DVD-ROM, zip disc, flash drive, videotape, audio tape, remote server, or other readable memory storage device. Detailed instructions need not be physically associated with the kit; instead, a user may be directed to an internet web site specified by the manufacturer or distributor of the kit, or supplied as electronic mail.

Designs for the kit include, but are not limited to, materials to quantify one or more RNAs determined to be differentially expressed as a result of the FSHD disease process, including but not limited to: miR-138, miR-486, miR-9, miR-32, miR-146b, miR-92a, miR-576, miR-142-3p, miR-505, miR-29b, miR-502-3p, miR-103, miR-98, miR-141, miR-34a, miR-140-3p, miR-100, miR-329, miR-454, miR-95, and/or miR-886-3p.

Designs for the kit may include, but are not limited to, materials to detect or quantify one or more proteins determined to be differentially expressed as a result of the FSHD disease process, including but not limited to: F13A1, IGF1, S100A8, PFN1, FBLN1, CFL1, TMSB4X, TPM4, EFEMP1, KRT16, SPP2, PROC, and/or PRG4.

Designs for the kit include, but are not limited to, materials to obtain, process, and/or extract biomarkers from patient samples, including materials to extract and/or store biofluids, separate components that make up biofluids, and/or extract or purify RNA, miRNA, or protein molecules from the samples.

Kit components for extraction of miRNA from a variety of different samples are known and are commercially available, for example from Qiagen, see worldwide web.qiagen.com/us/product-categories/discovery-and-translational-research/dna-rna-purification/rna-purification/mirna/(last accessed Nov. 1, 2020, incorporated by reference).

Kits for detecting miRNAs are commercially available and are incorporated by reference to hypertext transfer protocol secure://www.bing.com/search?q=detecting+mirna&src=IE-SearchBox&FORM=IESR3N (last accessed Oct. 20, 2020) or to worldwideweb.thermofisher.com/us/en/home/life-science/per/real-time-pcr/real-time-pcr-learning-center/real-time-per-basics/real-time-per-troubleshooting-tool/gene-expression-quantitation-troubleshooting/no-amplification/mirna- detection.html (last accessed Oct. 31, 2020). Such kits may be modified by replacement or inclusion of reagents suitable for detecting the miRNAs or marker proteins disclosed herein and/or with instructions for their use in detecting, diagnosing or monitoring FSHD.

Protein biomarkers may be detected by ELISA or other antibody-based assays using commercially available antibodies that detect the corresponding biomarker protein. Kits and kit components for purification of serum proteins including the biomarker proteins disclosed herein are known and commercially available, for example, from Norgen Biotek, see hypertext transfer protocol secure://norgenbiotek.com/product/abundant-serum-protein-depletion-kit (last accessed Nov. 1, 2020, incorporated by reference).

Kits for detection of proteins, such as proteins that are differentially expressed as a result of the FSHD disease process, including Staphylococcus protein A ELISA kits, are commercially available, for example, from ThermoFischer Scientific (worldwide web.thermofisher.com/us/en/home/life-science/antibodies/immunoassays/elisa-kits.html, last accessed Oct. 31, 2020, incorporated by reference).

Such kits may be customized to contain antibodies specific to FSHD associated proteins such as F13A1, IGF1, S100A8, PFN1, FBLN1, CFL1, TMSB4X, TPM4, EFEMP1, KRT16, SPP2, PROC, and/or PRG4. ELISAs include the various Invitrogen ELISA formats such as uncoated and antibody pair kits, coated ELISA kits, instant ELISA kits and ProQuantum kits and their equivalents. Antibodies, including monoclonal, monospecific or polyclonal antibodies that recognize or bind to one, two, three, four, five or more of these FSHD protein biomarkers may be incorporated into a kit for the detection or quantification of these biomarker proteins. Different kinds of ELISA suitable for detection of FSHD biomarker proteins are described below.

Direct ELISA. The steps of direct ELISA follow the mechanism below: A buffered solution of the antigen to be tested or detected (e.g. sample comprising a protein marker for FSHD) is added to each well usually 96-well plates of a microtiter plate, where it is given time to adhere to the plastic through charge interactions. A solution of nonreacting protein, such as bovine serum albumin or casein, is added to each well in order to cover any plastic surface in the well which remains uncoated by the antigen. The primary antibody with an attached (conjugated) enzyme is added, which binds specifically to the test antigen coating the well. A substrate for this enzyme is then added. Often, this substrate changes color upon reaction with the enzyme. The higher the concentration of the primary antibody present in the serum or other sample, the stronger is the color change. Often, a spectrometer is used to give quantitative values for color strength.

The enzyme acts as an amplifier; even if only few enzyme-linked antibodies remain bound, the enzyme molecules will produce many signal molecules. Within common-sense limitations, the enzyme can go on producing color indefinitely, but the more antibody is bound, the faster the color will develop. A major disadvantage of the direct ELISA is that the method of antigen immobilization is not specific; when serum is used as the source of test antigen, all proteins in the sample may stick to the microtiter plate well, and so small concentrations of analyte in serum must compete with other serum proteins when binding to the well surface. The sandwich or indirect ELISA provides a solution to this problem, by using a “capture” antibody specific for the test antigen to pull it out of the serum's molecular mixture

ELISA may be run in a qualitative or quantitative format. Qualitative results provide a simple positive or negative result (yes or no) for a sample. The cutoff between positive and negative is determined by the analyst and may be statistical. Two or three times the standard deviation (error inherent in a test) is often used to distinguish positive from negative samples. In quantitative ELISA, the optical density (OD) of the sample is compared to a standard curve, which is typically a serial dilution of a known-concentration solution of the target molecule. For example, if a test sample returns an OD of 1.0, the point on the standard curve that gave OD=1.0 must be of the same analyte concentration as the sample.

The use and meaning of the names “indirect ELISA” and “direct ELISA” differ in the literature and on web sites depending on the context of the experiment. When the presence of an antigen is analyzed, the name “direct ELISA” refers to an ELISA in which only a labelled primary antibody is used, and the term “indirect ELISA” refers to an ELISA in which the antigen is bound by the primary antibody which then is detected by a labeled secondary antibody. In the latter case a sandwich ELISA is clearly distinct from an indirect ELISA. When the “primary” antibody is of interest, e.g. in the case of immunization analyses, this antibody is directly detected by the secondary antibody and the term “indirect ELISA” applies to a setting with two antibodies.

Sandwich ELISA. A plate is coated with a capture antibody; a sample is added (e.g. containing a protein marker for FSHD), and any antigen present binds to capture antibody; a detecting antibody is added, and binds to antigen; an enzyme-linked secondary antibody is added, and binds to detecting antibody then substrate is added, and is converted by enzyme to detectable form. A “sandwich” ELISA is used to detect sample antigen. The steps are: A surface is prepared to which a known quantity of capture antibody is bound. Any nonspecific binding sites on the surface are blocked. The antigen-containing sample is applied to the plate, and captured by antibody. The plate is washed to remove unbound antigen. A specific antibody is added, and binds to antigen (hence the ‘sandwich’: the antigen is stuck between two antibodies). This primary antibody could also be in the serum of a donor to be tested for reactivity towards the antigen. Enzyme-linked secondary antibodies are applied as detection antibodies that also bind specifically to the antibody's Fc region (nonspecific). The plate is washed to remove the unbound antibody-enzyme conjugates. A chemical is added to be converted by the enzyme into a color or fluorescent or electrochemical signal. The absorbance or fluorescence or electrochemical signal (e.g., current) of the plate wells is measured to determine the presence and quantity of antigen. Without the first layer of “capture” antibody, any proteins in the sample (including serum proteins) may competitively adsorb to the plate surface, lowering the quantity of antigen immobilized. Use of the purified specific antibody to attach the antigen to the plastic eliminates a need to purify the antigen from complicated mixtures before the measurement, simplifying the assay, and increasing the specificity and the sensitivity of the assay. A sandwich ELISA used for research often needs validation because of the risk of false positive results.

Competitive ELISA. Another use of ELISA is through competitive binding. The steps for this ELISA are somewhat different from the first two examples: Unlabeled antibody is incubated in the presence of its antigen (sample). These bound antibody/antigen complexes are then added to an antigen-coated well. The plate is washed, so unbound antibodies are removed. (The more antigen in the sample, the more Ag-Ab complexes are formed and so there are less unbound antibodies available to bind to the antigen in the well, hence “competition”.) The secondary antibody, specific to the primary antibody, is added. This second antibody is coupled to the enzyme. A substrate is added, and remaining enzymes elicit a chromogenic or fluorescent signal. The reaction is stopped to prevent eventual saturation of the signal. Some competitive ELISA kits include enzyme-linked antigen rather than enzyme-linked antibody. The labeled antigen competes for primary antibody binding sites with the sample antigen (unlabeled). The less antigen in the sample, the more labeled antigen is retained in the well and the stronger the signal. Commonly, the antigen is not first positioned in the well.

One skilled in the art can select an appropriate enzymatic marker for ELISA. Enzymatic markers for ELISA include OPD (o-phenylenediamine dihydrochloride) turns amber to detect HRP (Horseradish Peroxidase), which is often used to as a conjugated protein. TMB (3,3′,5,5′-tetramethylbenzidine) turns blue when detecting HRP and turns yellow after the addition of sulfuric or phosphoric acid. ABTS (2,2′-Azinobis [3-ethylbenzothiazoline-6-sulfonic acid]-diammonium salt) turns green when detecting HRP. PNPP (p-Nitrophenyl Phosphate, Disodium Salt) turns yellow when detecting alkaline phosphatase.

In other embodiments, miRNA markers are detected or quantified. In one embodiment of such a method the at least one biomarker is one or more miRNAs that are dysregulated or quantitatively altered in subjects with or at risk of FSHD.

In another embodiment of this method the biomarker is one or more miRNA biomarkers selected from the group consisting of miR-9, miR-29b, miR-32, miR-34a, miR-92a, miR-98, miR-100, miR-103, miR-138, miR-140-3p, miR-141, miR-142-3p, miR-146b, miR-329, miR-454, miR-486, miR-502-3p, miR-505 and miR-576 or a variant thereof that may have 1 or 2 deletions, substitutions or additions of a nucleotide, and which binds to the same target site or crossblocks binding of the corresponding miRNA marker. Oligonucleotides complementary to one or more of these miRNA biomarkers may be incorporated into a kit for the detection or quantification of these biomarker miRNAS.

In one embodiment of this method the biomarker is selected from the group consisting of at least one of miR-100, miR-29b, miR-34a, miR-505 and miR-576. Oligonucleotides complementary to one or more of these miRNA biomarkers may be incorporated into a kit for the detection or quantification of these biomarker miRNAs.

In another embodiment of this method the biomarker is at least one miRNA selected from the group consisting miR-92a, miR-138, and miR-486, wherein the subject is selected as having or as at risk of having FSHD when one or more of these miRNAs is decreased or down-regulated compared to a control value; and/or wherein the biomarker is at least one miRNA selected from the group consisting of miR-9, miR-29b, miR-32, miR-142-3p, miR-146b, miR-505 and miR-576, wherein the subject is selected as having or as at risk of having FSHD when one or more of these miRNAs is increased or upregulated compared to a control value. Oligonucleotides complementary to one or more of these miRNA biomarkers may be incorporated into a kit for the detection or quantification of these biomarker miRNAs.

In another embodiment of this method, the biomarker is at least one miRNA selected from the group consisting of miR-140-3p and miR-502-3p, wherein the subject is selected as having or as at risk of having FSHD when one or more of these miRNAs is decreased or down-regulated compared to a control value; and/or wherein the biomarker is at least one miRNA selected from the group consisting of miR-29b, miR-32, miR-34a, miR-98, miR-100, miR-103, miR-141, miR-329, miR-454, and miR-505, wherein the subject is selected as having or as at risk of having FSHD when one or more of these miRNAs is increased or up-regulated compared to a control value. Oligonucleotides complementary to one or more of these miRNA biomarkers may be incorporated into a kit for the detection or quantification of these biomarker miRNAs.

In one embodiment, the biomarker comprises miR-100 and a kit for detecting this miRNA can comprise oligonucleotides complementary to miR-100. Similarly, kits for detecting the other miRNAs one or more of miR-140-3p and miR-502-3p; or complementary to one or more of miR-29b, miR-32, miR-34a, miR-98, miR-100, miR-103, miR-141, miR-329, miR-454, and miR-505.

In another embodiment, the biomarker comprises miR-502-3p, miR-95, and/or miR886-3p, wherein the subject has FSHD and the level of expression of the biomarker is indicative of the severity of the disease or may reflect the prognosis of the disease. Each of miR-95 and miR886-3p are increased in severe FSHD as compared to patients with mild FSHD; miR-502-3p is decreased in severe compared to mild FSHD patients; and miR-502-3p is downregulated in patients with severe FSHD as compared to healthy volunteers.

In another embodiment, each of miR-95 and miR886-3p are increased in severe FSHD as compared to patients with mild FSHD or normal controls; miR-502-3p is decreased in severe compared to mild FSHD patients or normal controls; and miR-502-3p is downregulated in patients with severe FSHD as compared to mild FSHD or healthy volunteers.

Another aspect of this technology is the method as disclosed above, wherein the biomarker is one or more protein biomarkers selected from the group consisting of F13A1, IGF1, S100A8, PFN1, FBLN1, CFL1, TMSB4X, TPM4, EFEMP1, KRT16, SPP2, PROC, and PRG4. As mentioned above, antibodies, including monoclonal, monospecific or polyclonal antibodies that recognize or bind to one, two, three, four, five or more of these FSHD protein biomarkers may be incorporated into a kit for the detection or quantification of these biomarker proteins

In some embodiments, the biomarker is one or more protein biomarkers selected from the group consisting of F13A1, IGF1, S100A8, PFN1, FBLN1, CFL1, TMSB4X, TPM4, EFEMP1, KRT16, and SPP2, and wherein said comparing comprises detecting an increase in expression of said protein biomarker(s) in a subject having or at risk of developing FSHD; and/or the biomarker is one or more protein biomarkers selected from the group consisting of PROC and PRG4 and wherein said comparing comprises detecting an decrease in expression of said protein biomarker(s) in a subject having or at risk of developing FSHD. The biomarker may also be at least one selected from the group consisting of IGF1, PRG4, PFN1, TPM4 and S100A8 and wherein said comparing comprises detecting an increase in expression of said at least one biomarker(s) in a subject having or at risk of developing FSHD.

In one embodiment, the biomarker is S100A8 wherein said comparing comprises detecting an increase in expression of S100A8 protein and/or calprotectin in a subject having or at risk of developing FSHD.

In another embodiment of the methods disclosed herein two or more biomarkers are detected which are selected from the groups consisting of miR-9, miR-29b, miR-32, miR-34a, miR-92a, miR-98, miR-100, miR-103, miR-138, miR-140-3p, miR-141, miR-142-3p, miR-146b, miR-329, miR-454, miR-486, miR-502-3p, miR-505 and miR-576, and F13A1, IGF1, S100A8, PFN1, FBLN1, CFL1, TMSB4X, TPM4, EFEMP1, KRT16, SPP2, PROC, and PRG4. For example, at least two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, or thirty two, biomarkers are detected. Such multiple biomarkers may be exclusively miRNA biomarkers, exclusively protein biomarkers or a mixture of both. Those skilled in the art may select an appropriate combination of biomarkers based on how a patient presents or select a combination of biomarkers that correlate best with FSHD in a particular patient or cohort of patients. Such methods may include detecting at least 2, 3, 4 or 5 of miR-100, miR-29b, miR-34a, miR-505 or miR-576; detecting at least 2, 3, 4, 5, 6, 7, 8, 9, 10, 11, 12 or 13 of S100A8, F13A1, IGF1, PFN1, FBLN1, CFL1, TMSB4X, TPM4, EFEMP1, KRT16, SPP2, PROC, and PRG4; or detecting at least 2, 3, 4, 5, 6, 7, 8, 9, 10, 11, 12, 13, 14, 15, 16, 17, or 18 of miR-100, miR-29b, miR-34a, miR-505, miR-576, S100A8, F13A1, IGF1, PFN1, FBLN1, CFL1, TMSB4X, TPM4, EFEMP1, KRT16, SPP2, PROC, and PRG4.

Some non-limited examples of marker combinations comprise or consist of miR-100 and miR-29b; miR-100 and miR-34a; miR-100 and miR-505; miR-100 and miR-576; miR-29b and miR-34a, miR-29b and miR-505 or miR-29b and miR-576; miR-34a and miR-505, or miR-34a and miR-576; and miR-576; or miR-505 or miR-576. These combinations may further comprise one other additional marker selected from the group consisting of miR-100, miR-29b, miR-34a, miR-505 and miR-576.

Other non-limited examples of marker combinations comprise or consist of miR-100, miR-29b, miR-34a, and miR-505; miR-100, miR-29b, miR-34a, and miR-576; miR-100, miR-29b, miR-505 or miR-576; miR-100, miR-34a, miR-505rand miR-576.

Other non-limited examples of marker combinations comprise or consist of S100A8 and F13A1, S100A8 and IGF1, S100A8 and PFN1, S100A8 and FBLN1, S100A8 and CFL1, S100A8 and TMSB4X, S100A8 and TPM4, S100A8 and EFEMP1, S100A8 and KRT16, S100A8 and SPP2, S100A8 and PROC, and S100A8 and PRG4.

Other non-limited examples of marker combinations comprise or consist of miR-100 and S100A8; miR-100 and one of F13A1, IGF1, PFN1, FBLN1, CFL1, TMSB4X, TPM4, EFEMP1, KRT16, SPP2, PROC, and PRG4.

In another embodiment of this technology, the method disclosed herein may further comprise treating the patient to prevent progression of, or to treat FSHD or ameliorate its symptoms. Treatments disclosed herein may be conducted in conjunction with detecting FSHD or independently.

In one embodiment, a subject is selected as having or being at risk of developing FSHD, mild FSHD, severe FSHD, FSHD-1 or FSHD-2 when one or more biomarkers disclosed herein are increased or decreased compared to control values.

In one embodiment, when a subject is selected as having or being at risk of developing FSHD, mild FSHD, severe FSHD, FSHD-1 or FSHD-2, then one or more treatments as disclosed herein are administered.

In one embodiment, the method further comprises, consists essentially or consists of administering to the selected subject an antisense oligonucleotide including those described by U.S. Ser. No. 16/649,122 or EU 18859092.1 which treatments are incorporated by reference to these documents.

Additional examples of specific treatment strategies include administration of antagomirs or antisense oligonucleotides; antisense oligos delivered via viral vectors, plasmids, bacteria, or nanoparticles; administration of small molecule inhibitors of miRNAs or proteins; protein replacement therapy; and use of antibody-based biologic treatments (e.g., Remicade and Humira) targeting the proteins such as S100A8. Other specific strategies are described by and incorporated by reference to Heier & Fiorillo, Patent Application Number PCT/US2019/035264 (WO2019232548), 2019, Compositions and methods for treating and preventing muscular disorders and dystrophies, steroid side effects, and inflammation.

In another embodiment, this method further comprises administering at least one miRNA selected from the group consisting of miR-9, miR-29b, miR-32, miR-34a, miR-92a, miR-98, miR-100, miR-103, miR-138, miR-140-3p, miR-141, miR-142-3p, miR-146b, miR-329, miR-454, miR-486, miR-502-3p, miR-505 and miR-576; or at least one inhibitor of said miRNAs such as oligonucleotides that hybridize to a corresponding mRNA and inhibit its function, degrade it or render it unavailable for binding to a target site, including RNA oligonucleotides comprising 2′-O methyl residues that confer increased binding affinity to RNA targets and resistance to endonuclease degradation or ZEN (naphthyl-azo) modifications that block exonuclease degradation.

In some embodiments, the method further comprises administering to the selected subject an agent that enhances epigenetic repression of D4Z4, targets DUX4 mRNA, blocks activity of the DUX4 protein or inhibits DUX4-induced processes leading to pathology; or further comprises administering to the selected subject Losmapimod or other selective inhibitor of p38α/β mitogen-activated protein kinases, antisense oligonucleotides that reduce DUX4 expression, or gene therapy, such as administration of miRNAs directed against DUX4. It may also further comprise administering to the selected subject an inhibitor of hyaluronic acid biosynthesis such as 4-methylumbellifoerone, a BET inhibitor, a casein kinase 1 inhibitor and/or vitamin C, vitamin E, acetylcysteine, zinc gluconate, selenomethionine or other antioxidants; or further comprise treating the selected subject for FSHD with surgical correction of facial weakness, scapular bracing, scapular fusion, scapuloplexy, tendon transfer such as pectoralis major transfer or Eden-Lange procedure or for the foot the Bridle procedure, correction of foot drop, ankle-foot orthoses, physiotherapy, occupational therapy, an assistive device, aerobic exercise, strength training, or cognitive behavioral therapy (CBT); or further comprise conducting an eye exam to identify retinal abnormalities, a hearing test to identify hearing loss, or pulmonary function testing to establish a baseline pulmonary function or changes from a prior established baseline.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIGS. 1A and 1B. DUX4 binding sites at loci surrounding miRNAs dysregulated in FSHD patients. The 19 miRNAs dysregulated in FSHD patient plasma samples were queried for potential DUX4 regulation using a DUX4 ChIP-seq dataset [22].

FIG. 1A: Overview of all DUX4 binding sites within regions capable of acting as regulatory elements (100 kb) of the 19 miRNAs and their home genes.

FIG. 1B: Schematic of DUX4 binding sites within the miR-100 locus and its surrounding home gene (MIR100HG) variants. Corresponding epigenetic modification maps display the location of histone modifications associated with active promoters (H3K4me3) and poised/active enhancers (H3K4me1 and H3K27Ac, respectively).

FIGS. 2A and 2B. Candidate miRNA loci are consistent with regulation via factors dysregulated by FSHD mutations.

FIG. 2A: Table listing a subset of transcription factors which are each increased in human skeletal muscle cells in response to DUX overexpression [22], along with the number of binding sites they show within potential regulatory distance (100 kb) of the 19 candidate miRNAs.

FIG. 2B: The miR-576 locus shows binding consistent with regulation by FOS, EGR1, MYC, YY1, and DUX4. Corresponding epigenetic modification maps display the location of histone modifications associated with active promoters (H3K4me3) and poised/active enhancers (H3K4me1 and H3K27Ac) in the vicinity of the miR-576 locus and its surrounding home gene, SEC24 homolog B (SEC24B). (DUX4 binding sites identified using ChIP-seq data uploaded from Geng et al [22]; additional transcription factor binding sites identified using UCSC Genome Browser and respective ChIP-seq datasets accessed via the Factorbook and ENCODE3 regulation tracks [23-27]).

FIG. 3. Pathway analysis of miRNAs and transcription factors dysregulated by FHSD mutations. Ingenuity Pathway Analysis software was used to identify established connections between candidate miRNAs from this study with transcription factors known to be dysregulated by FSHD-causing overexpression of DUX4 [22]. Red-shaded (light-speckled or diagonal stippling, e.g. miR-34a-5- and MYC) miRNAs and transcription factors were observed to increase, while those shaded blue (dark-speckled, or fine speckling); e.g. miR-501-3p and IRF1) were observed to decrease. Solid arrows denote direct relationships, while dashed arrows denote indirect relationships.

FIG. 4. Expression of candidate miRNAs in a validation group of FSHD patients. Candidate miRNAs that increased in the FSHD discovery experiment were assayed via individual qRT-PCR assay in a separate validation group of mild FSHD1 patient plasma samples. Expression levels of each miRNA are expressed as fold change versus healthy control volunteers. (values are mean±SEM, p≤0.05, one-tailed t-test comparing FSHD to control in direction of Discovery experiment; one outlier removed from miR-34a and miR-576 after significant Grubb's outlier test; n=7 healthy control volunteers, 12 mild FSHD).

FIG. 5. Validation and pathway analysis of elevated S100A8 protein in FSHD.

FIG. 5A: ELISA of S100A8 protein in plasma from a separate validation set of mild FSHD1 patients. Control, first bar, white. FSHD, second bar, speckled.

FIG. 5B: Bioinformatic pathway analysis was used to identify known connections between candidate protein markers with S100A8 pathway proteins involved in TLR4 signaling. PROC (fine speckles) and others in red (diagonal stippling and light speckling; e.g. S100A8 and TLR4).

FIG. 5C: Bioinformatic pathway analysis was used to identify established connections between candidate miRNAs with S100A8 pathway proteins involved in TLR4 signaling. miR-92a-30, miR-501-3p and mir-138 (blue, dark speckling), rest in red light speckles or diagonal stippling).

FIG. 5D: Bioinformatic analysis of binding sites for key S100A8 pathway transcription factors, AP-1 (FOS and JUN) and NF-κB (RELA), at potential regulatory regions of candidate miRNAs found to increase in FSHD plasma. Binding sites represent the combined number of potential promoter (within 2 kb of promoter) and enhancer (within 10 kb) regulatory regions with ChIP-seq-confirmed transcription factor binding for each miRNA home gene. (**p≤0.01; n=13 healthy control volunteers, 19 mild FSHD1; panels (b-c) produced using Ingenuity Pathway Analysis software, red=increased, blue=decreased).

### DETAILED DESCRIPTION OF THE INVENTION

The development of therapeutics for muscle diseases such as facioscapulohumeral dystrophy (FSHD) has been impeded by a lack of objective, minimally invasive biomarkers. The inventors have now identified blood-based biomarkers for FSHD such as circulating miRNAs and proteins that are dysregulated in early-onset FSHD patients. These biomarkers are decreased or increased in blood plasma of patients having FSHD as compared to values in sex and age matched subjects who do not have FSHD. More specifically, the inventors used low density quantitative PCR-based arrays to identify 19 such miRNAs and mass spectrometry proteomic analysis to identify 14 such proteins. Bioinformatic analysis of ChIP-seq data showed that the FSHD-dysregulated DUX4 transcription factor bound to regulatory regions of several candidate miRNAs. This panel of miRNAs also showed ChIP signatures consistent with regulation by additional transcription factors which are upregulated by FSHD-causing DUX4 mutations (FOS, EGR1, MYC, and YY1). Among other findings, validation studies in a separate group of mild FSHD patients showed consistent upregulation of miR-100, miR-103, miR-146b, miR-29b, miR-34a, miR-454, miR-505 and miR-576. An increase in expression of S100A8 protein, an inflammatory regulatory factor and subunit of calprotectin, was also validated by ELISA and bioinformatic analyses of proteomics and miRNA data further supported involvement of calprotectin and toll-like receptor 4 (TLR4) pathway dysregulation in FSHD.

A biofluid is defined as a biological fluid which is made by and/or can be extracted from the body or similar in vitro sources. Biofluids can be excreted by the body, extracted with a needle, or developed in response to a pathological process. A biofluid can be, but is not limited to, serum, plasma, or lymph; see Meng et al. Proteomic analysis of serum, plasma, and lymph for the identification of biomarkers. PROTEOMICS CLIN APPL, 2007 (incorporated by reference). Besides blood, plasma and serum, a biofluid can comprise bile, blood, breastmilk, cerebrospinal fluid, mucus, plasma, saliva, semen, serum, synovial (joint) fluid, stool, sweat, tears, or urine (Jordan et al. Semi-automated literature mining to identify putative biomarkers of disease from multiple biofluids. J CLIN BIOINFORMA, 2014.), or interstitial fluids (Nilsson et al. Lipid profiling of suction blister fluid: comparison of lipids in interstitial fluid and plasma. LIPIDS HEALTH DIS, 2019.), or peritoneal fluid (Lemoine et al. A validated inductively coupled plasma mass spectrometry (ICP-MS) method for the quantification of total platinum content in plasma, plasma ultrafiltrate, urine and peritoneal fluid. J PHARM BIOMED ANAL, 2018.), pleural or pericardial fluids (Provencio et al. Dynamic circulating tumor DNA quantification for the individualization of non-small-cell lung cancer patients treatment. ONCOTARGET, 2017.), or amniotic fluid (Orczyk-Pawilowicz et al. Metabolomics of Human Amniotic Fluid and Maternal Plasma during Normal Pregnancy. PLOS ONE, 2016.), or other bodily fluids. When a diagnostic procedure is performed to obtain a biofluid to detect molecular biomarkers, such as FSHD miRNA or protein markers, in the blood or other biofluids of a subject, this may be referred to as a liquid biopsy (Picher, Andy. Liquid Biopsy, Key for Precision Medicine” GENETIC ENGINEERING & BIOTECHNOLOGY NEWS. 23 Jul. 2018. Retrieved 12 Mar. 2019.) Each of the references above is incorporated by reference especially with regard to the source or mode of extraction or isolation of a biofluid.miR01

The term “FSHD” refers to “Facioscapulohumeral muscular dystrophy” which typically presents with weakness of the facial muscles, the stabilizers of the scapula, or the dorsiflexors of the foot. Severity is highly variable. Weakness is slowly progressive and approximately 20% of affected individuals eventually require a wheelchair. Life expectancy is not shortened. FSHD1 and FSHD2 are inherited in an autosomal dominant manner. In FSHD 1, approximately 70%-90% of individuals have inherited the disease-causing deletion from a parent, and approximately 10%-30% of affected individuals have FSHD as the result of a de novo deletion. Offspring of an affected individual have a 50% chance of inheriting the deletion. Prenatal testing for pregnancies at increased risk is possible if the D4Z4 pathogenic contraction has been identified in the family. Both FSHD1 and FSHD2 are inherited in a digenic manner.

FSHD1 represents about 95% of FSHDs and is characterized by a heterozygous pathogenic contraction of the D4Z4 repeat array in the subtelomeric region of chromosome 4q35 on the permissive chromosome 4 haplotype.

FSHD2 represents about 5% of FSHDs and is characterized by hypomethylation of the D4Z4 repeat array in the subtelomeric region of chromosome 4q35 on the permissive chromosome 4 haplotype due to one of the following: A heterozygous SMCHDJ pathogenic variant (<5% of individuals with FSHD; −85% of individuals with FSHD2); a heterozygous DNMT3B pathogenic variant (3 families reported); or unknown cause of hypomethylation of D4Z4 repeat array at 4q35 (2 families).

FSHD symptoms include inability to whistle; inability to sip through a straw; eyes that don't close fully during sleep; difficulty with sit-ups and pull-ups; shoulder blades that “wing” out; difficulty raising arm above shoulder height; weakness in hands and fingers; Foot drop (foot dorsiflexion weakness); weak lower abdominal muscles, “pregnant” belly; loss of chest (pectoral) muscles; curved spine (lordosis, kyphosis, scoliosis); chronic fatigue; and pain, often severe (reported in 70% of patients).

Severity of FSHD may be determined by those of skill in the art. One method for assessing severity is described by, and incorporated by reference to, Ricci, G., et al., J NEUROL. 2016; 263: 1204-1214 which describes Categories A1-A3, B1-B2, C1-C2 and D1-D2. Category A1 Severe facial weakness (unable both to close eyes and to protrude lips)+impairment of upper limb abduction with winged scapula (scapular FSHID score ˜1)+absence of uncommon features. Category A2: Facial weakness tapper and lower facial weakness) impairment of upper limb abduction with winged scapula (scapular FSHD score ≥1)+absence of uncommon features. Category A3: Facial weakness (upper or lower facial weakness)+impairment of upper limb abduction with winged scapula (scapular FSHD score ≥1)+absence of uncommon features, Category B1:impairment of upper limb abduction with winged scapula (scapular FSHD score ≥1) no facial weakness+absence of uncommon features. Category B2: facial weakness (facial FSHD score ≥1), no impairment of upper limb abduction+absence of uncommon features. Category C1: subject with presence of at least one typical sign FSHD score=0. Category C2: subject without signs of muscle weakness+FSHD score=0. Category D1: subject criterial of Categories A1, A2, A3, B1 or B2+at least one uncommon feature. Category D2: subject fulfilling criteria of categories C1 or C2-f at least one uncommon feature. Subject not fulfilling criteria of any of Categories A1 A3, B1-B2, C1-C2 or D1. An FSHD clinical score as described by, and incorporated by reference to, Lamperti, et al., MUSCLE NERVE, 2010, 42(2), 213-217 were used to classify subjects in Heier, et al., J Pers Med. 2020, 10(4):236, which reported the biomarker findings included in this application. In the study, the group with severe disease phenotypes is defined by having the disease severity score equal to or greater than 8; while the group with mild disease phenotypes is defined by having the disease severity score less than 8. These Classifications may be used to assess the mildness or severity of FSHD in a subject.

FSHD treatments include untargeted treatments such as drugs or regimens to improve muscle function through anabolic or anti-inflammatory effects. Treatments include those described by, and incorporated by reference to, Cohen, et al., TRENDS IN MOLECULAR MEDICINE, 2021, 27(2), 123-137. These include administration of β2-andrenergic agonists, which may be short-acting, long-acting, or ultralong acting, including drugs like albuterol or clenbuterol. Other such drugs or drug compositions may comprise one or more of bitolterol, fenoterol, isoprenaline, levosalbutamol, orciprenaline pirbuterol, procaterol, ritodrine, salbutamol, terbutaline, albuterol arformoterol, bambuterol, clenbuterol, formoterol, salmeterol, abediterol armoterol, indacaterol, olodaterol, vilanterol, isoxsuprein, mabuterol, and zilpaterol.

Other treatments include administration of glucocoticoids including prednisone, prednisolone, methylprednisolone, dexamethasone, betamethasone, tri am cinolone, fludrocotisone acetate and beclomethasone.

Aminoacyl-tRNA synthetases or physiocrines may be administered.

Testosterone or other anabolic steroids, or human growth hormone (HGH), rHGH (e.g., somatropin), HGH analogs or HGH releasers (e.g., GHRH) may be administered.

Creatine may be administered.

Antioxidants or nutraceuticals may be administered including flavonoids, omega-3 based compounds or other molecules exerting anti-inflammatory or anti-oxidant effects.

Targeted treatments such as those which reduce DUX4 expression, levels, or toxicity and gene therapy may also be used, such as administration of losmapimod a p38 mitogen-activated protein kinase inhibitor or other such inhibitors which inhibit p38alpha/beta MAPK-mediated signaling. Gene or stem cell therapies may also be employed.

Myostatin inhibitors including antibodies targeting myostatin like MYO-029 which are preferably humanized may be administered. Derivatives of follistatin such as the follastatin inhibitor ACE-083 may be administered.

Other modes of treatment for FSHD include an exercise program preferably directed by a professional, such as a physical or occupational therapist, who has experience with neuromuscular disorders. The program should emphasize exercising muscles that are still relatively strong and resting those that have weakened.

### Examples

Materials and Methods

Ethics Statement. Institutional approval for these clinical studies was obtained from the Institutional Review Board of Children's National Hospital and other participating Cooperative International Neuromuscular Research Group (CINRG) sites in accordance with all requirements. Where applicable, informed consent and/or assent was obtained from all patients or legal guardians before enrollment.

Patients and Sample Collection. Plasma samples were collected and biobanked from a previous early-onset FSHD study conducted by the Cooperative International Neuromuscular Research Group (CINRG) as described by, and incorporated by reference to, Mah et al [28].

For the discovery experiments, FSHD patients aged 10 to 51 years old were included (n=16 for miRNA discovery, n=25 for proteomics discovery), along with healthy control volunteers (n=8 for miRNA discovery, n=17 for proteomics discovery) aged 16 to 54 years old. All patients had Type 1 FSHD caused by epigenetic changes due to D4Z4 contraction which results in upregulation of DUX4.

miRNA Profiling. RNA was isolated and quantified from the discovery cohort of patients as incorporated by reference to, and as described previously by [29]. Briefly, RNA was isolated from 150 μl of plasma using Trizol LS reagent (ThermoFisher), then converted to cDNA using the High Capacity Reverse Transcription Kit with multiplexed RT primers (ThermoFisher). Synthesized cDNA was then preamplified using PreAmp MasterMix with multiplexed TM primers corresponding to the RT primers used in initial cDNA reaction. Quantitative analysis of miRNA was performed via TaqMan Low Density Array Cards (TAQMAN™ Array Human MicroRNA A Cards v2.0; ThermoFisher).

The ThermoFisher Cloud software suite with the Relative quantification (Rq) application was used to perform statistical analysis and determine expression of miRNA in either mild or severe FSHD patient groups versus healthy controls. A value >1 indicates an increase and a value <1 indicates a decrease in miRNA expression in FSHD versus healthy controls, with p-values ≤0.05 considered significant.

To reduce false-positive discovery in this setting, an evidence-based approach was used where candidate miRNAs that significantly increased in the discovery groups were cross-referenced to a separate set of non-overlapping CINRG patients used as a validation group.

Bioinformatics of miRNA Regulation via DUX4 and FSHD-associated Factors. Surrounding DNA regulatory regions of candidate miRNA genes were queried in ChIP-seq datasets for binding by transcription factors known to be impacted by FSHD. These analyses were performed using the UC Santa Cruz (UCSC) Genome Browser with alignment to the GECh37/hg19 genome build. For primary effects, due to the underlying mutation that causes FSHD, DUX4 binding was queried. For this, a user-supplied DUX4 ChIP-seq track published by, and incorporated by reference to, Geng et al was uploaded to determine which candidate miRNAs displayed physical binding of DUX4 at potential regulatory regions within 100 kb of the gene for each miRNA.

To investigate secondary factors whose dysregulation is associated with FSHD-causing mutations, DNA binding by transcription factors shown to be significantly up-regulated in cultured human muscle cells were investigated using microarray data by Geng et al [22]. For this, ChIP-seq data from the Encyclopedia of DNA Elements (ENCODE) [25,26] was used. From a master list of DUX4-regulated genes published in [22], a list of 34 transcription factors was identified with ChIP-seq data from ENCODE available within the UCSC Txn Factor ChIP Track and 47 transcription factors from the Txn Factor ChIP E3 Track [23,24,27]. After an initial survey of these full transcription factor lists for the 19 candidate miRNAs, a shorter focus list of 9 transcription factors whose binding was most frequently associated with the candidate miRNAs was narrowed down. DNA binding by transcription factors was queried in datasets produced using ChIP-seq from all 9 available cell line tracks, including GM12878 (lymphoblasts), H1-hESC (embryonic stem cells), HeLa-S3 (cervical cancer cells), HepG2 (liver cancer cells), HSMNI (skeletal muscle myoblasts), HUVEC (umbilical vein endothelial cells), K562 (immortalized myelogenous leukemia cells), NHEK (epidermal keratinocytes), and NHLF (lung fibroblasts).

In addition to binding by DUX4 and the transcription factors described above, ChIP-seq data for histone modifications were queried to gain insight into potential promoter or enhancer regulatory functions for the identified transcription factor binding sites. For this, histone H3K4 tri-methylation (found near promoters), H3K4 mono-methylation (found near regulatory elements), and H3K27 acetylation (found near active regulatory elements) were included. These histone modifications were queried in ChIP-seq datasets using all 9 available cell line tracks.

Pathway analysis was performed using Ingenuity Pathway Analysis software version 52912811. Candidate miRNAs from these studies were uploaded along with transcription factors whose dysregulation is associated with FSHD. Defined network connections were identified using the Pathway Builder application. Molecules confirmed to have established relationships were used to visualize a novel network built from these FSHD expression data.

Expression of individual miRNAs in a validation sample set. Circulating miRNAs that were significantly upregulated in patients having FSHD caused by DUX4-upregulating mutations were examined in a separate set of non-overlapping CINRG patients used as a validation group. For this cohort, FSHD patients were classified as having mild FSHD1 (n=12; 9 females, 3 males) and compared to healthy volunteer control samples (n=7; 4 females, 3 males). RNA was isolated from 150 μl of plasma using Trizol LS liquid extraction. Total RNA was converted to cDNA using a High Capacity Reverse Transcription Kit with multiplexed RT primers, preamplified using PreAmp MasterMix with multiplexed TM primers, and quantified with individual TaqMan assays on an ABI QuantStudio 7 real time PCR machine (Applied Biosystems; Foster City, CA).

Assay IDs used are: miR-32-002109, miR-103-000439, miR-505-002089, miR-146b-001097, miR-29b-000413, miR-34a-000426, miR-141-000463, miR-98-000577, miR-576-3p-002351, miR-9-000583, and miR-142-3p-000464. Expression levels of all miRNAs were normalized to the geometric mean of multiple control genes (miR-150 and miR-342-3p) determined previously to be stable circulating miRNA controls [30,31]. Expression was analyzed in FSHD versus healthy control patients via t-test analysis, including assessment of directionality. A p-value of <0.05 was considered significant. Data are presented as mean±SEM unless otherwise noted.

Proteomics profiling. Plasma samples were first processed using PIERCE™ Top12 Abundant Protein Depletion Spin Columns (Thermo Scientific) before mass spectrometry analyses using the Q Exactive HF mass spectrometer. Briefly, the 12 most abundant proteins from 5 μl of plasma sample were affinity depleted by incubating with Top12 protein depletion resin. Following this, the unbound fraction was collected according to the manufacturer's protocol. Proteins were precipitated with pre-cooled acetone (1:5 vol) for 30 minutes at −20° C. and centrifuged at 4° C. for 15 min at max speed in a micro-centrifuge. The liquid was decanted and the pellet was air dried briefly and resuspended with 8 M Urea, followed by reduction and alkylation with 5 mM DDT and 15 mM idodoacetamide for 30 min at room temperature. Samples were diluted with 100 mM Ammonia bicarbonate to final Urea concentration of less than 2 M. Afterwards, the samples were digested with 1 μg of trypsin (Promega) at 37° C. overnight. Trypsin was inactivated by 0.1% TFA and samples were desalted by capturing the peptides onto C18 100 ul bed tips (Pierce®C18 tips, Thermo Scientific) following the manufacture's protocol. The bound peptides were eluted with 60% Acetonitrile, 0.1% TFA, then dried using a SpeedVac, and resuspended in 20 μl buffer containing 2% Acetonitrile with 0.1% Acetic Acid.

The peptide mixtures from each fraction were sequentially analyzed by liquid chromatography tandem mass spectrometry (LC-MS/MS) using Thermo Ultimate 3000 RSLCnano—Q Exactive mass spectrometry platform nano-LC system (Easy nLC1000) connected to Q Exactive HF mass spectrometer (Thermo Scientific). This platform is configured with nano-electrospray ion source (Easy-Spray, Thermo Scientific), Acclaim PepMap 100 C18 nanoViper trap column (3 μm particle size, 75 μm ID×20 mm length), EASY-Spray C18 analytical column (2 μm particle size, 75 μm ID×500 mm length). The data from each sample was collected in triplicate at 2 μl per injection, following which the peptides were eluted at a flow rate of 300 nL/min using linear gradients of 7-25% Acetonitrile (in aqueous phase and Formic Acid) for 80 min, followed by to 45% for 25 min, and static flow at 90% for 15 min. The mass spectrometry data was collected in data-dependent manner switching between one full scan MS mode (m/z 380-1600, resolution 70,000, AGC 3e6) and 10 MS/MS mode (resolution 17,500); where MS/MS analysis of the top 10 target ions were performed once and dynamically excluded from the list for 30 seconds.

The MS raw data sets were searched against UniProt human database that included common contaminants using MaxQuant software (version 1.5.5.1) [32]. Default parameters were used for the searches, first search peptide tolerance 20 ppm, main search peptide tolerance 4.5 ppm, maximum two missed cleavage; and the peptide and resulting protein assignments were allowed at 0.01 FDR (thus 99% confidence level). Protein levels were quantified in 25 FSHD patients and 17 healthy controls and reported for each protein as the number of unique peptides detected and the intensity measured. Proteins with altered abundance with greater than 2-fold were selected for further inquiry.

Several pre-processing steps were performed on the raw data values before statistical analysis. Each sample had either 2 or 3 replicates which were averaged to yield a single quantification for each subject for each protein. When a value of zero occurs, it can indicate either a true zero or an assay that did not work properly for that protein. To accurately reflect protein levels, zeroes were incorporated into the analysis in the following way. If one replicate yielded a zero value, that zero was left as is and treated as a true zero. If two replicates yielded a zero, all values for that protein/sample were set to missing as we cannot distinguish true zeroes from artificial ones. A normalization factor was applied to the average values to account for differences in the amount assayed per sample. The protein counts were summed for all proteins for each sample and used the maximum value to normalize all other samples. This allowed us to ensure that the amount of proteins assayed were proportional for all samples.

All values were log-transformed for analysis. The relationship between protein levels and disease severity in the FSHD patients was assessed using a linear regression model where protein level was the dependent variable, severity was the independent variable, and age and gender were covariates. Regression models were performed only for proteins found in 5 or more samples. Model estimates were reported for each protein and included the coefficient and p-value for all terms in the model (severity, age and gender) along with an indication of the direction of each effect. This same method was used to assess the relationship between protein level and the number of D4Z4 repeats. The difference in protein expression was assessed between FSHD patients and healthy controls using a linear regression model where protein level was the dependent variable, a categorical indicator of disease was the independent variable, and age and gender were covariates. Again, regression models were performed only for proteins found in 5 or more samples. Model estimates were reported for each protein and included the coefficient and p-value for all terms in the model (disease status, age and gender), an indication of the direction of each effect, and age and gender adjusted means for each disease group. As this part of the analysis was discovery in nature, the resulting p-values for multiple testing were not adjusted. Our intention was to find those proteins showing some evidence of an effect and to move those proteins forward for an additional evidence-based validation experiment. The significance level for all analyses was set at 0.05.

Enzyme-linked immunosorbent assay (ELISA). Five proteins were chosen for further validation in a separate set of patients via protein-specific ELISA assays. Human specific protein ELISA kits for human insulin-like growth factor-1 (IGF1) (R&D), profilin 1 (PFN1) (LSBio), S100 Calcium Binding Protein A8 (S100-A8) (Biotechne), Proteoglycan 4 (PRG4) (AVIVA Systems Biology), Human Tropomyosin alpha-4 chain (TPM4) (MyBioSource) were performed to determine protein level in FSHD and unaffected controls. Plasma (20 μL) from mild FSHD1 patients (n=19) and healthy volunteer (n=13) controls (age and gender matched) were tested in duplicate following the manufacturer's recommended protocols. ELISA values were assessed for normality and a log-transformation applied where appropriate.

The relationship between protein level and severity was assessed using, as described above, a linear regression model where protein level was the dependent variable, severity was the independent variable, and age and gender were covariates.

The difference in protein expression between FSHD patients and healthy controls was assessed using a linear regression model where protein level was the dependent variable, a categorical indicator of disease was the independent variable, and age and gender were covariates. All analyses were performed at the 0.05 significance level.

Discovery of miRNA biomarkers associated with FSHD. Sixteen FSHD patients with pediatric onset, matched for sex and age, were selected into two groups of a discovery sample set for circulating biomarker studies: one mild FSHD group (n=8), and one severe FSHD group (n=8), as determined by an FSHD disease severity score. These two groups were each compared to a group of healthy control volunteers (n=8). Demographics are displayed in Table 1. Patients with severe FSHD showed a significantly higher FSHD severity score (12.25±2.76; p≤0.00001) than patients with mild FSHD (4.88±1.46), with any value of nine or higher being classified as severe FSHD.

TABLE 1
Clinical characteristics of study group patients

| | Healthy | | |
|---|---|---|---|
| | Control | Mild FSHD | Severe FSHD |
| N | 8 | 8 | 8 |
| Age in years | 28.29 ± 15.82 | 24.84 ± 10.46 | 27.58 ± 15.11 |
| (mean ± SD) | | | |
| Males:Females | 4:4 | 4:4 | 4: 4 |
| FSHD Severity Score | N/A | 4.88 ± 1.46 | 12.25 ± 2.76** |

**p ≤ 0.00001, t-test of mild FSHD versus severe FSHD severity score

Groups were age matched such that there was not a significant difference in age between each group and so that the mean ages did not differ significantly when examined by statistical analysis. Age and severity score data is summarized by Table 1B below.

| | | | Severity |
|---|---|---|---|
| | | Age | score |
| Mild | mean: | 24.84 | 4.88 |
| FSHD: | SD | 10.46 | 1.46 |
| | Median: | 21.38 | 5.00 |
| | t-test vs. healthy: | 0.61 | |
| Severe | mean: | 27.58 | 12.25 |
| FSHD: | SD | 15.11 | 2.76 |
| | Median: | 24.27 | 13.50 |
| | t-test vs. healthy: | 0.93 | |
| Healthy: | mean: | 28.29 | N/A |
| | SD | 15.82 | N/A |
| | Median: | 22.00 | N/A |

Ten miRNAs showed a significant change in expression level in mild FSHD plasma versus healthy controls, and twelve miRNAs showed a significant change in expression level in severe FSHD samples versus controls (Table 2). Of these, three miRNAs showed a significant increase in both mild and severe FSHD in comparison to healthy controls: miR-32, miR-505, and miR-29b. Each of these three miRNAs showed an approximately two-fold higher change in expression in severe FSHD patients than in mild FSHD patients versus healthy controls. Of the 19 unique miRNAs identified, several have been previously found to play a role in muscle disease pathways. miR-29b, which is associated with TGFβ-signaling and fibrosis, was upregulated in both mild and severe FSHD patients. Both miR-146b and miR-142-3p, which are known to be upregulated in inflammatory disease states, were upregulated in mild FSHD patients and have previously been shown to be upregulated in dystrophinopathy (Becker and Duchenne muscular dystrophy) patients and/or animal models [33,34]. miR-486 has previously been defined as a muscle-enriched microRNA or “myomiR” [35], and was found here to be downregulated in mild FSHD patients (p<0.005).

TABLE 2
Discovery of nineteen circulating miRNAs with
altered expression in mild or severe FSHD

| | | | | |
|---|---|---|---|---|
| | | P- | | |
| miRNA | ↑ or ↓ | value | Rq* | Known roles in muscle/disease pathways |

Mild FSHD versus healthy controls

| | | | | |
|---|---|---|---|---|
| 138 | ↓ | 0.004 | 0.05 | Heart development; hypoxia and S100A1 [36-38] |
| 486 | ↓ | 0.009 | 0.26 | myomiR; steroid-response in IBD blood [30, 35] |
| 9 | ↑ | 0.017 | 9.58 | Inhibits satellite cells; COPD weakness [39, 40] |
| 32 | ↑ | 0.020 | 8.45 | Cardiac fibrosis; VSMC calcification [41, 42] |
| 146b | ↑ | 0.034 | 2.18 | Upregulated in DMD and BMD [33, 34] |
| 92a | ↓ | 0.039 | 0.31 | Inhibits myogenic differentiation via Sp1 [43] |
| 576 | ↑ | 0.043 | 3.64 | Upregulated in smooth muscle tumors [44] |
| 142-3p | ↑ | 0.044 | 2.69 | Elevated in models of DMD and myositis [34, 45] |
| 505 | ↑ | 0.046 | 9.69 | Cardiac development and regeneration [46] |
| 29b | ↑ | 0.050 | 17.48 | Muscle atrophy, therapeutic target [47, 48] |

Severe FSHD versus healthy controls

| | | | | |
|---|---|---|---|---|
| 32 | ↑ | 0.001 | 17.09 | Cardiac fibrosis; VSMC calcification [41, 42] |
| 505 | ↑ | 0.007 | 19.51 | Cardiac development and regeneration [46] |
| 502-3p | ↓ | 0.009 | 0.36 | Myogenic differentiation; ACAD marker [49, 50] |
| 103 | ↑ | 0.013 | 4.29 | Myogenic differentiation [50] |
| 98 | ↑ | 0.014 | 21.65 | Muscle differentiation [51] |
| 141 | ↑ | 0.016 | 7.52 | Biomarker for prostate and bladder cancer [52] |
| 29b | ↑ | 0.018 | 28.78 | Muscle atrophy, therapeutic target [47, 48] |
| 34a | ↑ | 0.024 | 8.12 | Up in FSHD and myotonic dystrophy [53, 54] |
| 140-3p | ↓ | 0.028 | 0.54 | Plasma biomarker of myotonic dystrophy [55, 56] |
| 100 | ↑ | 0.029 | 3.58 | Upregulated in LMNA dystrophy biopsies [57] |
| 329 | ↑ | 0.030 | 4.63 | Counteracts muscle hypertrophy [58] |
| 454 | ↑ | 0.046 | 2.02 | Plasma biomarker of myotonic dystrophy [55, 56] |

Italics = dysregulated in both mild and severe FSHD; ACAD = acute coronary artery disease, BMD = Becker muscular dystrophy, COPD = chronic obstructive pulmonary disease, DMD = Duchenne muscular dystrophy, IBD = inflammatory bowel disease, LMNA = Lamin A/C, TGFβ = Transforming Growth Factor β, VSMC = vascular smooth muscle cell.

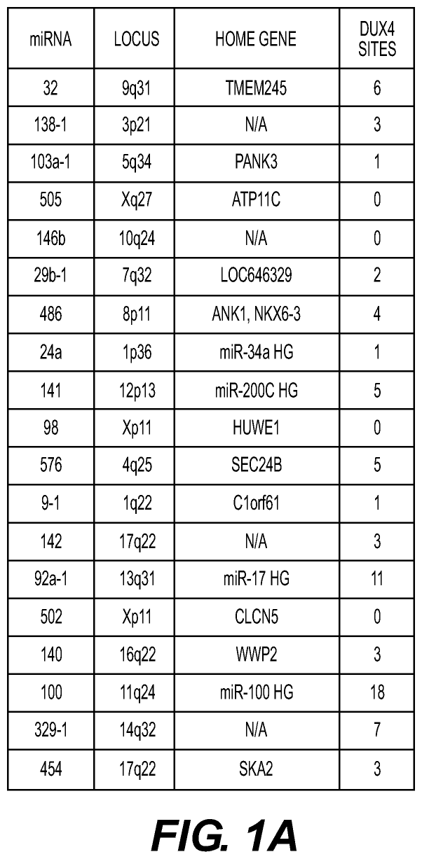

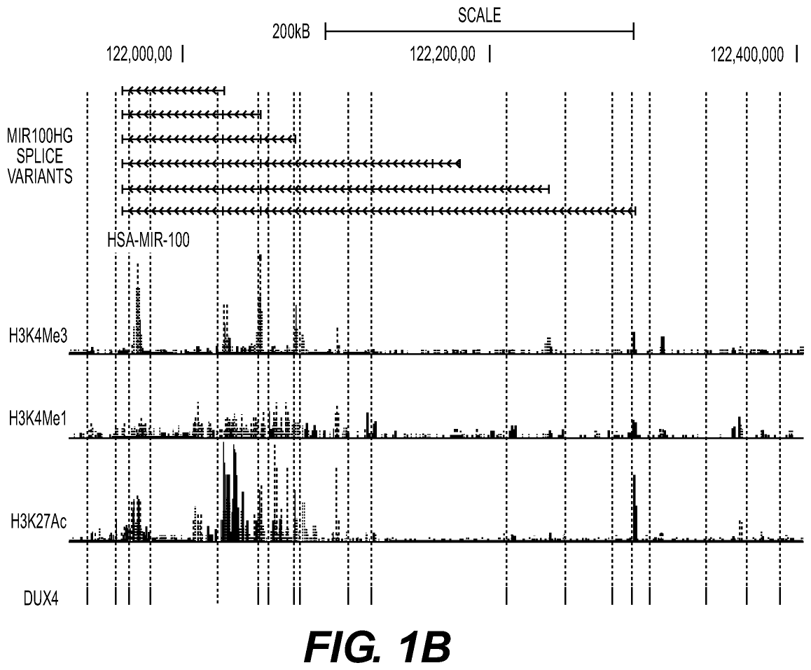

Bioinformatic analysis of miRNA regulation and pathways. To examine their regulation by transcription factors which are dysregulated by the FSHD disease process, a bioinformatic analyses of ChIP-seq data for DNA binding by transcription factors in proximity to each candidate miRNA's genomic locus was performed. To gain insight into direct consequences of DUX4 mutations that cause FSHD, ChIP-seq data for DUX4 (FIG. 1) was analyzed. Genes for sixteen of the candidate miRNAs had at least one binding site within distances capable of providing gene enhancer functions. Examination of the miR-100 home gene (MIR100HG) locus was particularly interesting. In total, we found 18 DUX4 binding sites in the area surrounding MIR100HG, and many of these clearly overlapped with histone modifications associated with active promoters (H3K4 tri-methylation) and regulatory elements (H3K27Ac). These data are consistent with regulation of miR-100 expression by DUX4 (FIG. 1B).

To gain insight into additional pathways that may drive expression of candidate miRNAs and contribute to FSHD molecular pathophysiology, bioinformatic analyses of ChIP-seq data for transcription factors that are dysregulated as a result of DUX4 mutations was performed. For this, a list of transcription factors which are expressed at significantly different levels in human skeletal muscle cells as a result of DUX4 overexpression was obtained. Of the transcription factors in this dataset, 34 had ChIP-seq datasets available in the Factorbook repository and 47 had ChIP-seq datasets available in the Encode 3 repository. Genomic binding by each of these transcription factors was surveyed for each of these transcription factors for all candidate miRNAs (Table S1).

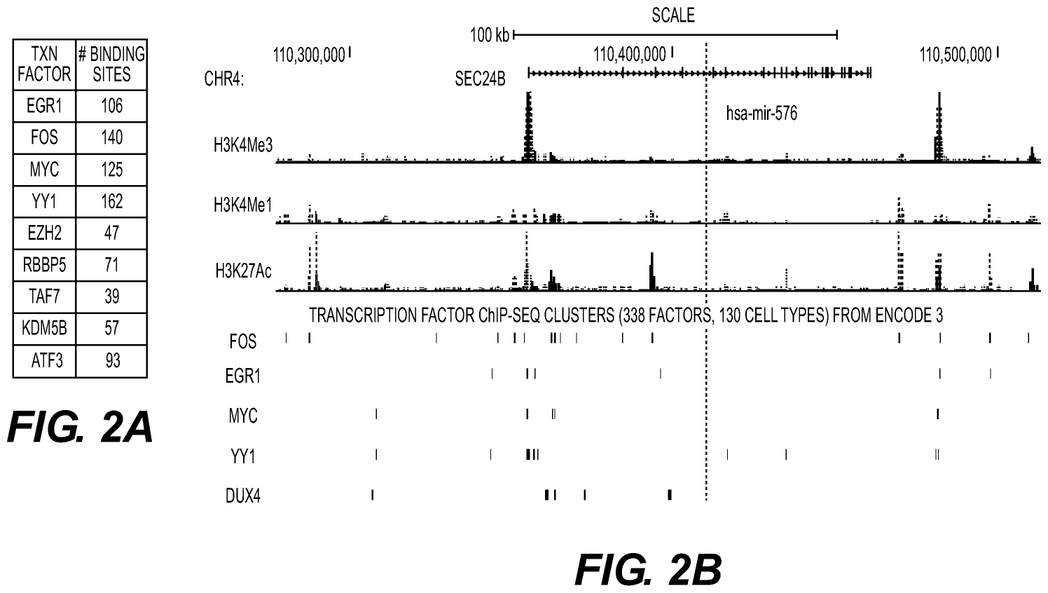

Transcription factors that were increased in response to overexpression of toxic, full-length DUX4 but not increased in response to a non-toxic, truncated isoform of DUX4 were considered to be of particular interest (FIG. 2A). Of these factors, four showed a particularly high number of binding sites within regulatory distance of the candidate miRNAs: EGR1, FOS, MYC, and YY1. As an example of these findings, miR-576 was upregulated in FSHD patients, has five DUX4 binding sites neighboring its home gene (SEC24B), and has a high number of binding sites for the secondary transcription factors described here (FIG. 2B). EGR1, FOS, MYC and YY1 all showed a large number of binding sites around miR-576, and these frequently overlapped with histone modifications which mark active promoter and enhancer regions, consistent with these four transcription factors driving gene expression signatures in FSHD.

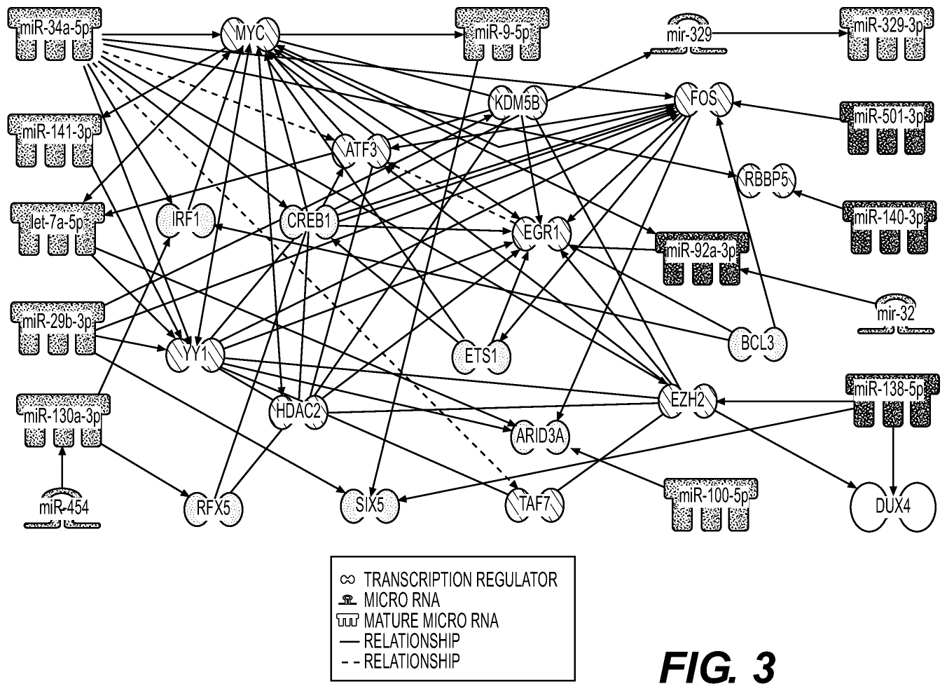

Additionally, a bioinformatic pathway analysis was performed on the candidate miRNAs and transcription factors previously known to be dysregulated in FSHD, to see if there are defined signaling pathways or interactions shared by these factors. Interestingly, this analysis showed that there are previously established connections between many of the miRNAs and transcription factors examined, with 15 of the miRNAs and 18 of the transcription factors found to make up a network with previously defined interactions (FIG. 3). Together, these bioinformatics data show our candidate miRNA markers are consistent with a change in transcriptional programming that results from FSHD-causing DUX4 overexpression mutations.

Confirmation of miRNA increases in mild FSHD patients. Next, expression of candidate miRNA biomarkers was assayed in samples from a separate and non-overlapping group of patients. Upon clinical examination, all patients in this validation group were determined to have mild FSHD. Fourteen miRNAs that significantly increased in the discovery experiments for follow-up study in the validation group were selected. Three of these miRNAs (miR-9, miR-32 and miR-329) were not expressed at consistently high enough levels for detection within plasma from the validation cohort of mild FSHD patients, leaving 11 miRNAs for validation. Here, these 11 individual candidate miRNAs were quantified in mild FSHD (n=12; 9 females, 3 males) versus healthy volunteer control samples (n=7; 4 females, 3 males).

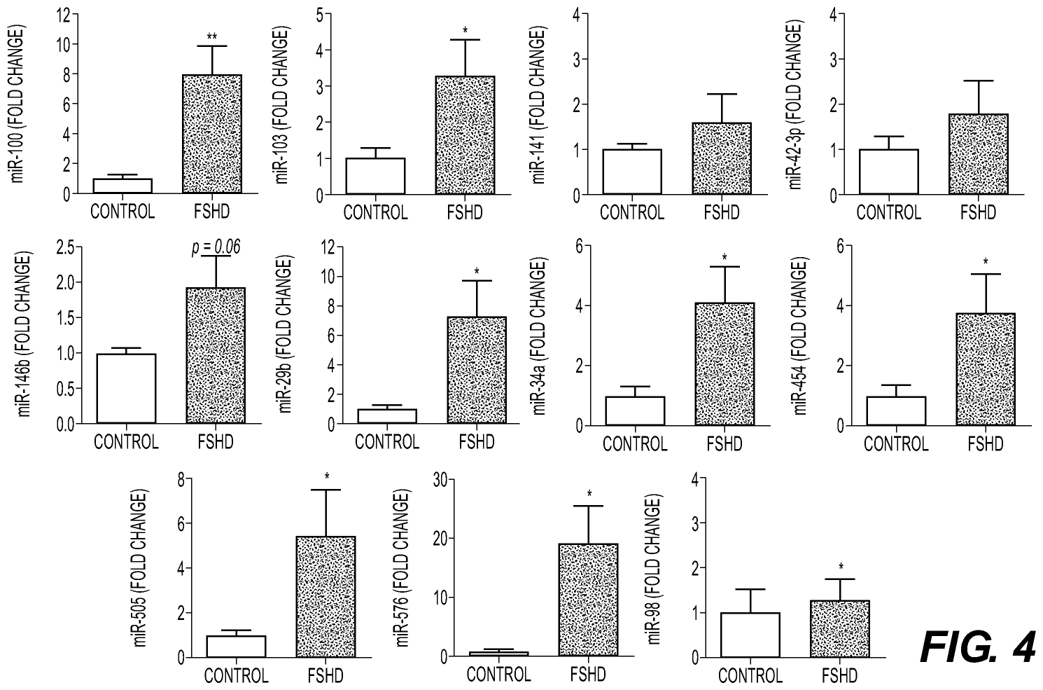

Upon quantification, we found 8 of these 11 candidate miRNAs also showed a clear increase in samples from the FSHD validation cohort in comparison to healthy controls (FIG. 4). miR-100, miR-103, miR-29b, miR-34a, miR-454, miR-505 and miR-576 were all expressed at significantly higher levels (p≤0.05) in FSHD serum. miR-100, miR-29b, miR-34a, miR-505 and miR-576 were the most highly upregulated in FSHD, showing upregulation from approximately 4- to 20-fold higher than healthy controls.

miR-146b was also expressed at an approximately 2-fold higher level in this set of FSHD patients, however it did not reach significance (p=0.06). Of the remaining three miRNA candidates, miR-98 showed no apparent change, while miR-141 and miR-142-3p showed an approximately fifty percent increase that did not reach significance. As a majority of candidate miRNAs showed consistent behavior in this separate validation set of mild FSHD samples, this panel of miRNAs merits further investigation as biomarkers moving forward.

Proteomics profiling. To identify protein candidate biomarkers, we performed LC-MS/MS based proteomic profiling of samples from a discovery group of FSHD patients (Table 3). For this, plasma from FSHD patients (n=25) was compared to healthy volunteer controls (n=17), with a roughly even mix of males and females, and an average age of early- to mid-twenties for each group. All FSHD patients were confirmed to have FSHD1 resulting from D4Z4 contraction mutations that alter epigenetic regulation of DUX4.

TABLE 3
Clinical characteristics of patients in proteomics discovery group

| | Healthy Control | FSHD |
|---|---|---|
| N | 17 | 25 |
| Age in years (mean ± SD) | 23.45 ± 13.18 | 25.68 ± 14.71 |
| Males:Females | 9:8 | 13:12 |
| FSHD Severity Score | N/A | 8.54 ± 4.10 |

Based on signal intensity, 33 proteins that were significantly different between FSHD and healthy control samples were identified (Table S2).

TABLE S2
Circulating proteins identified as dysregulated in FSHD plasma via LC-MS/MS, based on signal intensity

| | | | Total Unique | ↑ | | |
|---|---|---|---|---|---|---|
| | | Gene | peptides N | or | | |
| UniProt ID | Protein name | Name | (Control/FSHD) | ↓ | p-value | Known roles in muscle/disease pathways |
| Q15848 | Adiponectin | ADIPOQ | 12(5/7) | ↑ | 0.0142 | increased in DMD; adipokine that regulates |
| | | | | | | metabolism in muscle |
| P04114 | Apolipoprotein B-100 | APOB | 39(14/25) | ↓ | 0.0167 | lipid transport, elevated in heart disease |
| P55056 | Apolipoprotein C-IV | APOC4 | 20(9/11) | ↓ | 0.0139 | lipid transport from intestine to muscle |
| P02655 | Apolipoprotein C-II | APOC2 | 33(13/20) | ↓ | 0.0058 | lipid transport; genetic marker for myotonic |
| | | | | | | dystrophy |
| P23528 | Cofilin-1 | CFL1 | 18(8/10) | ↑ | 0.0037 | actin filament organization and depolymerization |
| P02741 | C-reactive protein | CRP | 11(3/8) | ↑ | 0.0328 | elevated in myositis; elevated/biomarker for IBD |
| P01034 | Cystatin-C | CST3 | 23(10/13) | ↓ | 0.0283 | biomarker for cardiovascular and kidney diseases |
| Q12805 | Fibulin-3 | EFEMP1 | 13(5/8) | ↑ | 0.0013 | plasma biomarker for mesothelioma; retinal |
| P03951 | Coagulation factor XI | F11 | 30(12/18) | ↓ | 0.0381 | Noonan syndrome & hypotonia; near D4Z4 |
| | | | | | | genomic locus; coagulation factor |
| P00488 | Coagulation factor | F13A1 | 13(5/8) | ↑ | 0.0227 | hypertension, angiotensin II, coagulation |
| | XIII A chain | | | | | |
| P23142 | Fibulin-1 | FBLN1 | 23(9/14) | ↑ | 0.0037 | positive regulation of fibroblast proliferation |
| P00738 | Haptoglobin | HP | 39(14/25) | ↑ | 0.0485 | up in DMD plasma; associated with IBD, arthritis |
| | | | | | | and other inflammatory diseases |
| P05019 | Insulin-like growth | IGF1 | 26(11/15) | ↑ | 0.0398 | hypertrophy, development, satellite cells, |
| | factor I | | | | | regeneration |
| P01857 | Ig gamma-1 chain C | IGHG1 | 27(10/17) | ↓ | 0.0262 | down in endothelial corneal dystrophy |
| | region | | | | | |
| P03952 | Plasma kallikrein | KLKB1 | 38(14/240 | ↓ | 0.0408 | inflammation and coagulation; near D4Z4 |
| P07737 | Profilin-1 | PFN1 | 19(7/12) | ↑ | 0.0003 | actin cytoskeleton organization |
| Q92954 | Proteoglycan 4; | PRG4 | 23(8/15) | ↓ | 0.0377 | TRL4; anti-inflammatory, down in arthritis |
| P04070 | Vitamin K-dependent | PROC | 22(9/13) | ↓ | 0.0376 | anti-inflammatory, down in chronic inflammatory |
| | protein C | | | | | diseases such as IBD |
| P41222 | Prostaglandin-H2 D- | PTGDS | 15(7/8) | ↑ | 0.0463 | neuromodulator; smooth muscle contraction |
| | isomerase | | | | | |
| P61224 | Ras-related protein | RAP1B | 15(6/9) | ↑ | 0.0111 | GTP-binding protein |
| | Rap-1b, 1a | | | | | |
| P05109 | Protein S100-A8 | S100A8 | 18(5/13) | ↑ | 0.0042 | TLR4; pro-inflammation, up in rheumatic diseases |
| | | | | | | and IBD |
| P0DJI9 | Serum amyloid A-2 | SAA2 | 5(1/4) | ↑ | 0.0339 | IBD, Induce Pathogenic Th17 Cells |
| | protein | | | | | |
| Q13103 | Secreted | SPP2 | 13(7/6) | ↑ | 0.0431 | pro-inflammatory, NF-κB; blood pressure; bone |
| | phosphoprotein 24 | | | | | health |
| P37802 | Transgelin-2 | TAGLN2 | 15(7/8) | ↑ | 0.0416 | marker of differentiated smooth muscle |
| P60174 | Triosephosphate | TPI1 | 8(2/6) | ↑ | 0.0479 | glycolysis |
| | isomerase | | | | | |
| P67936 | Tropomyosin alpha-4 | TPM4 | 17(8/9) | ↑ | 0.0421 | actin organization, muscle contraction |
| | chain | | | | | |
| Q6EMK4 | Vasorin | VASN | 23(9/14) | ↓ | 0.0481 | binds TGF-β; vascular smooth muscle |
| P15924 | Desmoplakin | DSP | 16(7/9) | ↓ | 0.020 | down in mdx muscle; intercellular junctions; |
| P29401 | Transketolase | TKT | 6(2/4) | ↑ | 0.030 | connects pentose phosphate pathway to glycolysis |
| F5H7V9 | Tenascin | TNC | 4(1/3) | ↓ | 0.041 | extracellular matrix, adhesion modulation |
| O95497 | Pantetheinase | VNN1 | 10(4/6) | ↑ | 0.008 | upregulated in IBD |
| P08779 | Keratin, type I | KRT16 | 13(7/6) | ↑ | 0.009 | elevated with S100A8 in skin disorders, psoriasis |
| | cytoskeletal 16 | | | | | |

DMD = Duchenne muscular dystrophy, IBD = inflammatory bowel disease, mdx = mouse model for Duchenne muscular dystrophy X-linked.

To further filter the protein list, unique peptide count data was used to identify proteins that had significantly different counts between FSHD and control samples. This narrowed the candidates down to 14 proteins (Table 4); among these, twelve proteins were higher in FSHD samples versus healthy controls, while two proteins were lower in the FSHD samples versus healthy controls.

TABLE 4
Fourteen circulating proteins identified as dysregulated in FSHD plasma via LC-MS/MS

| Gene | UniProt | | p- | |
|---|---|---|---|---|
| Name | ID | ↑ or ↓ | value | Known roles in muscle/disease |
| F13A1 | P00488 | ↑ | 0.031 | Hypertension, angiotensin II, coagulation |
| IGF1 | P05019 | ↑ | 0.043 | hypertrophy, development, satellite cells, regeneration |
| S100A8 | p05109 | ↑ | 0.009 | TLR4; pro-inflammation, up in rheumatic diseases [59-62] |
| PFN1 | P07737 | ↑ | 0.010 | actin cytoskeleton organization |
| FBLN1 | p23142 | ↑ | 0.011 | positive regulation of fibroblast proliferation |
| CFL1 | P23528 | ↑ | 0.031 | actin filament organization and depolymerization |
| TMSB4X | P62328 | ↑ | 0.017 | actin filament organization |
| TPM4 | P67936 | ↑ | 0.015 | actin organization, muscle contraction |
| EFEMP1 | Q12805 | ↑ | 0.001 | plasma biomarker for mesothelioma; retinal dystrophy [63] |
| KRT16 | P08779 | ↑ | 0.009 | Elevated with S100A8 in skin disorders, psoriasis [60, 64-66] |
| SPP2 | Q13103 | ↑ | 0.017 | Pro-inflammatory, NF-κB; blood pressure; bone health [67] |
| PROC | P04070 | ↓ | 0.048 | anti-inflammatory, down in chronic inflammation [68, 69] |
| PRG4 | Q92954 | ↓ | 0.024 | TLR4; anti-inflammatory, down in arthritis [70, 71] |

CFL1 = Cofilin 1, EFEMP1 = EGF-containing fibulin-like extracellular matrix protein 1, F13A1 = Coagulation factor XIII A chain, FBLN1 = fibulin-1, IBD = inflammatory bowel disease, IGF1 = Insulin-like growth factor 1, KRT16 = Keratin 16, PFN1 = Profilin-1, PRG4 = Proteoglycan 4 or lubricin, PROC = Protein C, S100A8 = S100 calcium-binding protein A8, SPP2 = Secreted phosphoprotein 24, TLR4 = Toll-like receptor 4, TMSB4X = Thymosin beta-4, TPM4 = Tropomyosin alpha-4 chain.

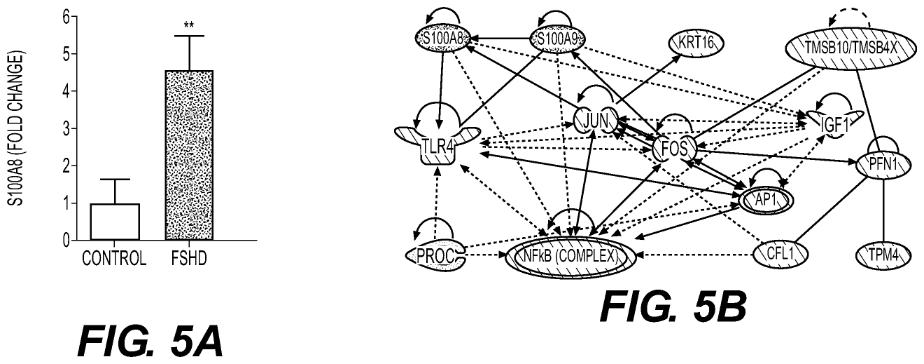

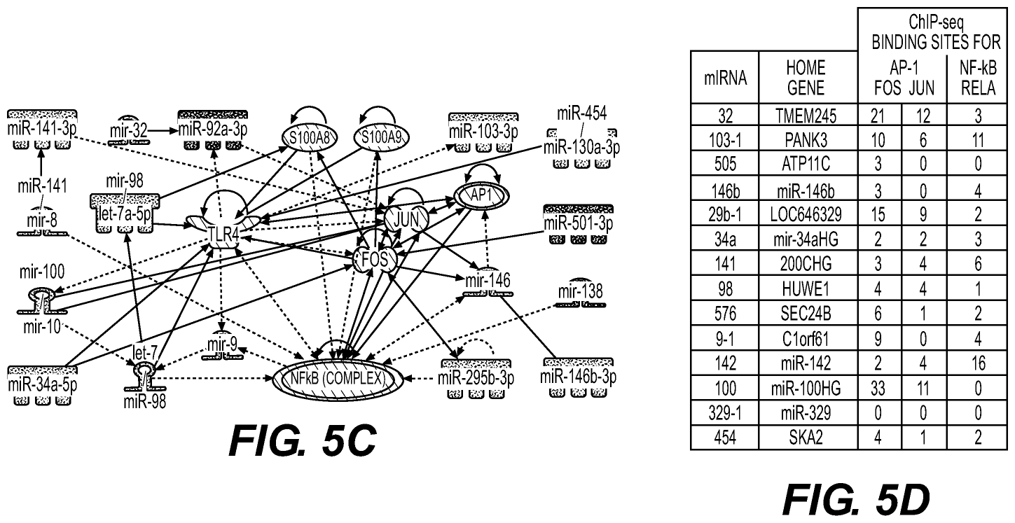

Five candidate protein markers were selected for subsequent quantification via protein-specific ELISA analysis of a non-overlapping validation group of FSHD samples. These included Insulin-Like Growth Factor 1 (IGF1), proteoglycan 4 (PRG4), profilin 1 (PFN1), tropomyosin 4 (TPM4), and S100 calcium-binding protein A8 (S100A8). Of these candidate proteins, S100A8 showed a significant increase in FSHD plasma of approximately 4.5-fold over healthy controls in the validation group (FIG. 5A), consistent with its behavior in the discovery experiment. To determine if elevated S100A8 signaling was consistent with the overall proteomic and miRNA profiling results, bioinformatic pathway analyses was performed focused on the S100A8 pathway along with the full list of candidate protein (FIG. 5B) and miRNA (FIG. 5C) markers. Nine proteins and thirteen miRNAs were shown to have previously established connections to the toll-like receptor 4 (TLR4) signaling pathway, which is activated by S100A8 and drives increased inflammatory (NF-κB and AP-1) gene expression. As miRNAs can reflect a direct readout of transcription factor activity, we also surveyed ChIP-seq data to analyze DNA regions encoding miRNAs elevated in FSHD for binding by the NF-κB and AP-1 transcription factors activated by S100A8 (FIG. 5D). All miRNAs except for one (miR-329) showed binding by NF-κB and/or AP-1 subunits at DNA regions capable of acting as regulatory promoter or enhancer elements. As S100A8 is a well-established biomarker of inflammatory disease processes (reviewed in [61]) and these can be upregulated in the muscular dystrophies, this protein merits further investigation as a biomarker for FSHD.

Treatments for FSHD remain elusive. However, research advances in FSHD are now beginning to yield promising and novel therapeutic strategies that will require well-designed clinical trials to evaluate effectiveness. Potential therapeutic strategies including antisense oligonucleotides (AON) and small molecules have been reported or are being actively pursued [72-75]. Changes in biomarkers following a treatment can be a powerful tool for evaluating the efficacy and safety of the treatment. Previous studies seeking to identify circulating miRNA biomarkers in muscular dystrophy have focused exclusively on assaying myomiRs, which are a defined group of miRNAs with muscle-specific or muscle-enhanced expression [76,77]. Previously, a study by Statland et al identified 7 potential protein biomarkers in 22 FSHD serum samples, using a commercial multiplex assay [78]. A multi-site study using aptamer-based SomaScan proteomics to assay two FSHD populations identified a total of 115 proteins that were dysregulated, four of which behaved consistently between the two independent cohorts (creatine kinase M M, creatine kinase 1\4B, carbonic anhydrase III, and troponin I type 2) [79]. In this work omics approaches were applied to identify additional circulating miRNA and protein biomarker candidates using samples collected from individuals with early-onset FSHD.

Circulating miRNAs offer many advantages as biomarkers in diseases affecting muscle, as they are stable, objective, minimally invasive, and well-conserved between human patients and preclinical animal models. Here the inventors identify eight circulating miRNAs that were associated with FSHD in patient plasma samples. The prevalence of DNA binding by DUX4 and FSHD-associated transcription factors, within regions capable of regulating the candidate miRNAs, provides a molecular rationale for their upregulation in FSHD. Several of the markers have also been previously shown to play a role in muscle diseases and associated pathological pathways. These biomarkers may be used for monitoring biomarkers in early-onset FSHD.

Several candidate miRNAs we identified have previously been proposed as circulating biomarkers and have shown similar behavior in other diseases. Plasma miR-454 has been identified as a biomarker of myotonic dystrophy [55,56]. Serum miR-146b is a pharmacodynamic biomarker in inflammatory bowel disease (IBD) [29,30]. Intriguingly, miR-146b is also known to downregulate dystrophin in multiple muscle diseases, is increased in dystrophinopathies and in myositis, and is also drug-responsive in the mdx mouse model of DMD [33,45]. Urinary miR-141 provides a promising diagnostic biomarker for the identification of both prostate and bladder cancers [52]; it will be interesting to determine if this or other candidate miRNAs are also dysregulated in urine from dystrophic patients, as this sampling method could provide a completely non-invasive biomarker.

Increases in circulating S100A8, a subunit of calprotectin, were consistent with an inflammatory signature playing a role in FSHD. The inflammatory calprotectin protein consists of a heterodimer (S100A8/S100A9) which binds to toll-like receptor 4 (TLR4) to activate pro-inflammatory gene expression pathways through the NF-κB and AP-1 transcription factors. Consistent with such an inflammatory gene signature in FSHD, bioinformatic analyses here showed five of the candidate miRNAs have established connections with TLR4 signaling, are increased in FSHD patients, and have gene promoters that are bound by AP-1 and/or NF-κB.

Outside of FSHD, calprotectin is already a well-established biomarker across rheumatic diseases. Fecal calprotectin is a widely used diagnostic, monitoring and pharmacodynamic biomarker for IBD, and recent studies indicate serum calprotectin levels are also well-correlated with IBD disease state [62,80]. Serum calprotectin is used as a monitoring and pharmacodynamic biomarker for rheumatoid arthritis, and intriguingly S100A8/S100A9 may have further utility in arthritis as a molecular imaging marker of inflammatory activity [59,81,82]. Of particular relevance to the present work, calprotectin in both muscle and serum is a biomarker for disease activity in juvenile dermatomyositis [83]. The use of S100A8 or calprotectin as completely non-invasive or local biomarkers for FSHD and other muscle diseases such as myositis is proposed.

Several of the molecular markers we identified here as elevated in FSHD may provide a new therapeutic target. In various states of muscle atrophy miR-29b is also upregulated, while preventing its expression shows efficacy in mouse models of muscle atrophy [47,48]. In myositis and Becker muscular dystrophy, the inflammatory marker miR-146b is known to downregulate dystrophin expression, whereas the reduction of miR-146b via anti-inflammatory drugs or via miRNA-targeting oligos is proposed as a method to increase dystrophin levels to help improve muscle health [33,45]. In various rheumatological disease states, the inhibition of S100A8 or calprotectin via small molecule inhibitors or antibodies is a very attractive therapeutic strategy; early studies of such inhibitors are already showing therapeutic efficacy in both human trials and/or in mouse models, including in studies for arthritis, asthma, IBD, and multiple sclerosis (reviewed in [61]). Similarly, decreases in PROC seen here in FSHD are also seen in a number of rheumatological disorders, where treatment with PROC activators are already being pursued as a therapeutic option (reviewed in [69]).

Bioinformatic analyses of the -omics results support muscle and inflammatory gene expression pathways as being dysregulated in FSHD. A number of muscle pathology-associated miRNAs are dysregulated in FSHD patients: miR-486 is a defined myomiR, miR-29b upregulation promotes muscle atrophy, miR-146b is dysregulated in dystrophinopathies and myositis, miR-329 counteracts muscle hypertrophy, and three others are known to be dysregulated in myotonic dystrophy, lamin A (LMNA) dystrophy, and/or FSHD (miR-34a, miR-140-3p, miR-100, and miR-454). Consistent with these findings, several of the proteins that were dysregulated are known to function in muscle contraction, actin filament organization and/or muscle regeneration (TOM4, PFN1, CFL1, TMSB4X and IGF1).

S100A8 and its associated inflammatory signaling pathway (TLR4, NF-κB and AP-1) appear to be a substantial hub for dysregulated expression of the candidate markers we identified. Nine of the candidate miRNAs have previously established connections to this TLR4-centered pathway. ChIP-seq analysis of the miRNAs upregulated in FSHD shows all but one have promoters bound by NF-κB or AP-1, which are activated by S100A8-induced TLR4. In the proteomics data, several of the proteins that increased are pro-inflammatory (S100A8, KRT16 and SPP2) while in contrast the two proteins that decreased have anti-inflammatory (PROC and PRG4) roles. Consistent with our FSHD findings, KRT16 and S100A8 were also upregulated together in inflammatory skin disorders; additionally, the pattern of increased S100A8 with decreased PROC is seen here in FSHD as well as in IBD and a number of other chronic inflammatory disorders [68,69]. Pathway analysis further establishes a link between the protein markers, as nine out of fourteen have established connections to the S100A8 and TLR4 signaling pathway. Together these data confirm that circulating FSHD biomarkers reflect muscle pathogenesis, and suggest inflammatory S100A8/TLR4 signaling plays a role in pediatric onset FSHD as well.

FSHD is chronic genetic muscle disease with a variable prognosis. There is no cure, and no pharmaceuticals for FSHD have shown efficacy in altering the disease course. Development of objective biomarkers will facilitate the clinical and preclinical development of novel therapies, as well as our ability to monitor disease activity.

Among others, eight circulating miRNAs (miR-100, miR-103, miR-146b, miR-29b, miR-34a, miR-454, miR-505, and miR-576) were identified as biomarkers for FSHD. Additionally, the S100A8 subunit of calprotectin was identified as a primary protein marker of interest for FSHD, consistent with its utility in numerous rheumatic diseases. These molecular markers also will be useful for further investigation of additional cohorts, preclinical drug testing, and for clinical trials.

Table S1 (appended) describes the ChIP-seq of FSHD-disrupted transcription factors at candidate miRNA loci. Table S1 is an integral part of this disclosure.

Table S2 (above) describes proteomic changes in plasma from patients with FSHD.

Table S1 (appended) describes the ChIP-seq of FSHD-disrupted transcription factors at candidate miRNA loci.

To build a biological rationale for the changes in biomarker expression that we observed in FSHD plasma samples, the molecular mechanisms by which those biomarkers are regulated at the genomic level were investigated. ChIP-seq is a method that identifies precise DNA sequences in the genome that are physically bound by a transcription factor protein in order to regulate the expression levels of nearby genes. If the level of a transcription factor is increased by the FSHD disease process, then molecular biomarkers (e.g. miRNAs and proteins) whose expression is regulated by that transcription factor will also likely be changed. To investigate this, we performed a bioinformatic analysis where we queried publicly available ChIP-seq data to detect DNA binding sites for each transcription factor that are in close proximity to the biomarkers we identified as significantly changed in FSHD plasma. The list of transcription factors we queried were selected from a previous publication (Geng et al. “DUX4 activates germline genes, retroelements, and immune mediators: implications for facioscapulohumeral dystrophy.” DEV CELL, 2012, incorporated by reference.) which reported a list of transcription factors which show altered expression as a result of DUX4 overexpression, consistent with the cause of FSHD. A second criterion for our list of transcription factors was that they must have ChIP-seq data available through the UCSC genome browser “Factorbook” or “ENCODE 3” tracks. The data from this bioinformatic analysis showed that the genomic loci around many of the miRNA biomarkers we identified as altered in FSHD samples are indeed consistent with their regulation by the same transcription factors upregulated by the FSHD disease process. For example, the proteins FOS, MYC and YY1 are all upregulated by DUX4 overexpression and all showed >100 binding sites within regions capable of regulating FSHD miRNA biomarkers we identified. Consistent with regulation by transcription factors disrupted in FSHD, the genomic loci for many of the miRNA biomarkers we identified also showed a large number of binding sites by these transcription factors, such as miR-100 which showed over 70 binding sites for these transcription factors. Together, these findings are consistent with a model where DUX4 overexpression causes FSHD, then transcription factors are dysregulated by the FSHD disease process, this dysregulation of transcription factors then causes significant changes in the expression of molecular markers (e.g. miRNAs and proteins) which can be detected in biofluids, and we can thus detect/quantify levels of these molecules in biofluids or liquid biopsies to use them as biomarkers indicative of FSHD disease pathology.

### Terminology

Terminology used herein is for the purpose of describing particular embodiments only and is not intended to limit the invention.

Unless defined otherwise, all technical and scientific terms used herein have the same meaning as commonly understood by one of ordinary skill in the art to which this invention belongs. In case of conflict, the present application including the definitions will control.

Nomenclature for miRNAs is known in the art and is incorporated by reference to Griffiths-Jones, et al., miRBase: microRNA sequences, targets and gene nomenclature, NUCLEIC ACIDS RES. 2006 Jan. 1; 34 (Database issue): D140-D144. The registry for miRNA sequences, miRBase (hypertext transfer protocol://worldwideweb.mirbase.org), is constantly updated. Unless otherwise specified the sequences of the miRNAs disclosed herein are incorporated by reference to the last version of miRBase prior to the effective filing date of this application.

As used herein, the term “microRNA” (“miRNA) refers to a small (e.g., 10-50 nucleotide) RNA (or nucleotide analogs) which can be genetically encoded or synthetically produced and is capable of directing or mediating RNA silencing. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing with the miRNA and most commonly results in translational inhibition or destabilization of the target mRNA. In some embodiments, a variant of a particular miRNA will have insertions, substitutions, or deletions of one or two bases of the miRNA and bind to the same target site as the referent miRNA. Unless otherwise specified the miRNAs disclosed herein are human and may be prefixed with “hsa-”. In some embodiments, an immature microRNA, such as one having a step-loop structure, corresponding to a particular miRNA, may be used instead of a mature form.

As used herein, a “miRNA disorder” refers to a disease or disorder characterized by an aberrant expression or activity of a miRNA.

Commercial genetic tests are available for FSHD Type 1 and Type 2. Other tests for FSHD include blood tests to measure levels of serum creatine kinase (CK), an enzyme that is released into the bloodstream when muscle fibers are deteriorating, and serum aldolase, an enzyme that helps break down sugars into energy. Elevated levels of either of these enzymes can indicate a problem with muscles and a need for additional testing. However, a normal CK level does not rule out FSHD; neurological tests to rule out other nervous system disorders, identify patterns of muscle weakness and wasting, test reflexes and coordination, and detect muscle contractures; muscle biopsies, which involve the removal of muscle tissue using a biopsy needle or during a simple surgical procedure. The tissue is then examined under a microscope. In FSHD, a muscle biopsy might reveal several abnormalities, but none are uniquely characteristic for the disease, or the muscle might even appear normal. To confirm a diagnosis of FSHD with certainty, a genetic test is needed.

Such tests may be used in conjunction with the invention to diagnose or monitor FSHD or to correlate the biomarkers disclosed herein with FSHD or its different forms or patient populations. Severity of FSHD can be determined by one skilled in the art using an FSHD severity score, such as that described by and incorporated by reference to Mah, et al., Cooperative International Neuromuscular Research Group I. A multinational study on motor function in early-onset FSHD. NEUROLOGY 2018; 90: e1333-e1338 or by Lamperti C, et al., A standardized clinical evaluation of patients affected by facioscapulohumeral muscular dystrophy: the FSHD clinical score. MUSCLE NERVE 2010; 42:213-217.

Alternatively, severity scores may be determined as described by and incorporated by reference to: hypertext transfer protocol secure://worldwideweb.urmc.rochester.edu/MediaLibraries/URMCMedia/fields-center/documents/ClinicalSeverityScoring.pdf (last accessed Oct. 20, 2020).

Lamperti, et al., MUSCLE NERVE, 2010, 42(2), 21.3-217 criteria were used to classify subjects in Heier, et al., Pers Med. 2020, 10(4):236, which reported the biomarker findings included in this application. The disease severity scale consists of continuous numbers from 1 to 15, where 1 indicates the mildest disease severity and 15 indicates the most severity disease presentation. In Heier, et al., J Pers Med. 2020, 10(4):236, the group with severe disease phenotypes is defined by having the disease severity score equal to or greater than 8; while the group with mild disease phenotypes is defined by having the disease severity score less than 8.

An age-matched control subject may be within ±0, 1, 2, 3, 4, 5, 6, 7, 8, 9, or 10 years of age as subject being assessed for FSHD.

Preferably, groups are age matched such that there was not a significant difference in age between each group and so that the mean ages did not differ by more than 5 years.

Gender matched subjects are matched based on biological sex as male or female.

The terms “increased” or “decreased” describe the relative levels of a biomarker as compared to a control value which may be an age and gender matched control or an average control value obtained from a larger cohort or population of subjects not having FSHD. Increases or decreases may range from <5, 5, 10, 20, 30, 40, 50, 100, 150, 200, 250, 300% or more (or any intermediate subrange or value) of the control value, such as a level of the same biomarker in an age and gender matched subject.

The terms “down-regulated” and “up-regulated” describe decreases or increases in the rate of expression, rate of degradation and generally in the rate of decrease or increase of a biomarker compared to a control value.

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, intrans, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after synthesis or polymerization, such as by conjugation with a labeling component.

In the context of this disclosure, the term “oligonucleotide” also refers to a plurality of nucleotides joined together in a specific sequence from naturally and nonnaturally occurring nucleobases. Nucleobases of the disclosure are joined through a sugar moiety via phosphorus linkages, and may include any one or combination of adenine, guanine, cytosine, uracil, thymine, xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, other aza and deaza thymidines, other aza and deaza cytosines, other aza and deaza adenines, other aza and deaza guanines, 5-trifluoromethyl uracil and 5-trifluoro cytosine. The sugar moiety may be deoxyribose or ribose. The sugar moiety may be a modified deoxyribose or ribose with one or more modifications on the C1, C2, C3, C4, and/or C5 carbons.

“Modified oligonucleotide” means an oligonucleotide having one or more modifications relative to a naturally occurring terminus, sugar, nucleobase, and/or internucleoside linkage. A modified oligonucleotide may comprise unmodified nucleosides at one or a plurality of any of the positions of the disclosed nucleic acids. The oligonucleotides of the disclosure may also comprise modified nucleobases or nucleobases having other modifications consistent with the spirit of this disclosure and in particular modifications that increase their nuclease resistance in order to facilitate their use as therapeutic, diagnostic or research reagents. A modified oligonucleotide may also carry one or more epigenetic modifications.

Real-Time qRT-PCR (Real-Time Quantitative Reverse Transcription PCR) is a major development of PCR technology that enables reliable detection and measurement of products generated during each cycle of PCR process. This technique became possible after introduction of an oligonucleotide probe which was designed to hybridize within the target sequence. Cleavage of the probe during PCR because of the 5′ nuclease activity of Taq polymerase can be used to detect amplification of the target-specific product. qRT-PCR techniques useful for characterizing miRNAs are known in the art and are incorporated by reference to hypertext transfer protocol secure://wordwideweb.ncbi.nlm.nih.gov/probe/docs/techqper/ (last accessed Oct. 21, 2020) or to Heid, C A, et al., Quantitative Real Time PCR. GENOME RES 1996, 6:986-994.

In some embodiments, a regression model can be used to identify the significantly associated miRNAs targeting a set of candidate genes, mRNAs, or genetic or metabolic pathways frequently involved in FSHD. Multiple linear regression analysis can be used to construct the model and find the significant mRNA-miRNA associations; see Fengfeng Wang, et al., Multiple Regression Analysis of mRNA-miRNA Associations in Colorectal Cancer Pathway, BIOMED RES INT. 2014; 2014: 676724 (incorporated by reference) or Yan Yao, et al., Integrative Analysis of miRNA and mRNA Expression Profiles Associated With Human Atrial Aging, FRONT. PHYSIOL., 19 Sep. 2019 hypertext transfer protocol secure://doi org/10.3389/fphys.2019.01226 (incorporated by reference).

ThermoFisher Cloud software was used to analyze the real-time qRT-PCR miRNA data. This uses a ddCt method to analyze expression levels of the miRNAs by normalizing them to the geometric mean of multiple control genes (miR-150 and miR-342-3p were chosen as the control normalization genes) and then normalizing this to a control sample or group (healthy volunteers) in the experiment. The logarithmic ddCt (or DDCq) value is then converted to a Relative quantification (Rq) value that is a linear value of the test group (severe or mild FSHD) in comparison to controls (healthy), in a manner that is consistent with the widely used 2\hat{ }(-ddCt) method. This method is described by, and incorporated by reference to, Livak K J, & Schmittgen T D. Analysis of relative gene expression data using real-time quantitative PCR and the 2(-Delta Delta C(T)) Method. METHODS, 2001, 25: 402-408.

The terms “polypeptide”, “peptide” and “protein” are used interchangeably herein to refer to polymers of amino acids of any length including peptide fragments such as those disclosed herein. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The enzyme-linked immunosorbent assay (ELISA) is a commonly used analytical biochemistry assay which can be used to detect proteins or protein fragments associated with FSHD status. The assay uses a solid-phase type of enzyme immunoassay (EIA) to detect the presence of a ligand (commonly a protein) in a liquid sample using antibodies directed against the protein to be measured. Antibodies to the proteins or protein fragments described herein are known in the art and commercially available. ELISA methodology and materials are also incorporated by reference to Crowther, J. R. “Chapter 2: Basic Principles of ELISA”. ELISA: Theory and Practice. METHODS IN MOLECULAR BIOLOGY. 1995, 42. Humana Press. pp. 35-62.

As used herein, the term “isolated nucleic acid or oligonucleotide” or “isolated protein or peptide” refers to molecules which are substantially free or functionally free of other cellular material, or culture medium when produced by recombinant techniques, or substantially free of chemical precursors or other chemicals when chemically synthesized.

Treatment,” or “treating,” as used herein, is defined as the application or administration of a therapeutic agent, such as an miRNA, protein or peptide, enzyme, or drug, as disclosed herein deficient in a FSHD patient, or an inhibitor of a miRNA or protein over-expressed in an FSHD patient such as an RNA or oligonucleotide complementary to an over-expressed miRNA, to a patient, or application or administration of a therapeutic agent to an isolated tissue or cell line from a patient, who has or at risk of developing FSHD or a symptom of FSHD, with the purpose to prevent, cure, heal, alleviate, relieve, alter, remedy, ameliorate, improve or affect FSHD or a symptom thereof.

These therapeutic agents can be incorporated into pharmaceutical compositions suitable for administration. Such compositions typically comprise the active agent such as an oligonucleotide that binds to a specific miRNA or an antibody that binds to or neutralizes a specific protein biomarker, and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

A pharmaceutical composition of the invention is formulated to be compatible with its intended route of administration. Examples of routes of administration include parenteral, e.g., intravenous (iv), intradermal, subcutaneous (sc), intraperitoneal, intramuscular (im), oral, respiratory (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). Preferably, the composition should be sterile and stable and may contain inhibitors of nucleases such as RNAses for oligonucleotides or protease inhibitors for proteinaceous agents like antibodies. The carrier can be a solvent or dispersion medium containing, for example, water, ethanol, polyol (for example, glycerol, propylene glycol, and liquid polyethylene glycol, and the like), and suitable mixtures thereof.

Sterile injectable solutions can be prepared by incorporating the active compound in the required amount in an appropriate solvent, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Oligonucleotide agents, such as those complementary to over-expressed miRNAs, can also be administered by transfection or infection using methods known in the art, including but not limited to the methods described in McCaffrey et al., NATURE, 2002, 418(6893), 38-9 (hydrodynamic transfection); Xia et al., NATURE BIOTECHNOL., 2002, 20(10), 1006-10 (viral-mediated delivery); or Putnam, AM. J. HEALTH SYST. PHARM. 1006, 53(2), 151-160, erratum at AM. J. HEALTH SYST. PHARM. 1996, 53(3), 325.

Therapeutic agents can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., CLIN. IMMUNOL. IMMUNOPATHOL., 1998, 88(2), 205-10. Liposomes (e.g., as described in U.S. Pat. No. 6,472,375) and microencapsulation can also be used. Biodegradable targetable microparticle delivery systems can also be used, e.g., as described in U.S. Pat. No. 6,471,996. All of the above documents are incorporated by reference for the methods and reagents they disclose.

In one embodiment, the therapeutic agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted with monoclonal antibodies to tissues affected by FSHD) can also be used as pharmaceutically acceptable carriers. These can be prepared according to methods known to those skilled in the art, for example, as described in, and incorporated by reference to, U.S. Pat. No. 4,522,811.

Toxicity and therapeutic efficacy of a therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a kit, container, pack or dispenser together with equipment and optional instructions for administration.

Any of the methods of treatment disclosed herein may be used to treat a subject at risk of, newly diagnosed as having, or previously diagnosed as having FSHD, mild FSHD or severe FSHD. Alternatively, they may be performed in conjunction with detecting or monitoring FSHD status by the methods disclosed herein.

Unless otherwise required by context, singular terms shall include pluralities and plural terms shall include the singular. All ranges provided within the application are inclusive of the values of the upper and lower ends of the range unless specifically indicated otherwise.

The term “and/or” as used in a phrase such as “A and/or B” herein is intended to include “A and B”, “A or B”, “A”, and “B”.

“About” means either within 10% of the stated value, or within 5% of the stated value, or in some cases within 2.5% or 1% of the stated value, or, “about” can mean rounded to the nearest significant digit.

Reference to properties or compositions that are “substantially the same” or “substantially identical” indicates minor and irrelevant deviations that are not material to the characteristics considered important in the context of the invention. In various embodiments this can mean the properties are within 10%, and preferably within 5%, within 2.5% or within 1% of the reference value.

The materials, methods, and examples are illustrative only and are not intended to be limiting. Other features and advantages of the invention will be apparent from the description and from the claims.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

### REFERENCES

1. van Overveld P G, Lemmers R J, Sandkuijl L A, Enthoven L, Winokur S T, Bakels F, Padberg G W, van Ommen G J, Frants R R, van der Maarel S M. Hypomethylation of D4Z4 in 4q-linked and non-4q-linked facioscapulohumeral muscular dystrophy. Nat Genet 2003; 35: 315-317. Epub 23 Nov. 2023.
2. Dixit M, Ansseau E, Tassin A, Winokur S, Shi R, Qian H, Sauvage S, Matteotti C, van Acker A M, Leo O, Figlewicz D, Barro M, Laoudj-Chenivesse D, Belayew A, Coppee F, Chen Y W. DUX4, a candidate gene of facioscapulohumeral muscular dystrophy, encodes a transcriptional activator of PITX1. Proc Natl Acad Sci USA 2007; 104: 18157-18162. DOI: 10.1073/pnas.0708659104
3. Lemmers R J, van der Vliet P J, Klooster R, Sacconi S, Camano P, Dauwerse J G, Snider L, Straasheijm K R, van Ommen G J, Padberg G W, Miller D G, Tapscott S J, Tawil R, Frants R R, van der Maarel S M. A unifying genetic model for facioscapulohumeral muscular dystrophy. Science 2010; 329: 1650-1653. DOI: 10.1126/science.1189044
4. Lemmers R J, Tawil R, Petek L M, Balog J, Block G J, Santen G W, Amell A M, van der Vliet P J, Almomani R, Straasheijm K R, Krom Y D, Klooster R, Sun Y, den Dunnen J T, Helmer Q, Donlin-Smith C M, Padberg G W, van Engelen B G, de Greef J C, Aartsma-Rus A M, Frants R R, de Visser M, Desnuelle C, Sacconi S, Filippova G N, Bakker B, Bamshad M J, Tapscott S J, Miller D G, van der Maarel S M. Digenic inheritance of an SMCHD1 mutation and an FSHD-permissive D4Z4 allele causes facioscapulohumeral muscular dystrophy type 2. Nat Genet 2012; 44: 1370-1374. DOI: 10.1038/ng.2454
5. van den Boogaard M L, Lemmers R, Balog J, Wohlgemuth M, Auranen M, Mitsuhashi S, van der Vliet P J, Straasheijm K R, van den Akker R F P, Kriek M, Laurense-Bik M E Y, Raz V, van Ostaijen-Ten Dam M M, Hansson K B M, van der Kooi E L, Kiuru-Enari S, Udd B, van Tol M J D, Nishino I, Tawil R, Tapscott S J, van Engelen B G M, van der Maarel S M. Mutations in DNMT3B Modify Epigenetic Repression of the D4Z4 Repeat and the Penetrance of Facioscapulohumeral Dystrophy. Am J Hum Genet 2016; 98: 1020-1029. DOI: 10.1016/j.ajhg.2016.03.013
6. Hamanaka K, Sikrova D, Mitsuhashi S, Masuda H, Sekiguchi Y, Sugiyama A, Shibuya K, Lemmers R, Goossens R, Ogawa M, Nagao K, Obuse C, Noguchi S, Hayashi Y K, Kuwabara S, Balog J, Nishino I, van der Maarel S M. Homozygous nonsense variant in LRIF1 associated with facioscapulohumeral muscular dystrophy. Neurology 2020; 94: e2441-e2447. DOI: 10.1212/WNL.0000000000009617
7. Sharma V, Harafuji N, Belayew A, Chen Y W. DUX4 differentially regulates transcriptomes of human rhabdomyosarcoma and mouse C2C12 cells. PLoS One 2013; 8: e64691. DOI: 10.1371/journal.pone.0064691
8. Geng L N, Yao Z, Snider L, Fong A P, Cech J N, Young J M, van der Maarel S M, Ruzzo W L, Gentleman R C, Tawil R, Tapscott S J. DUX4 Activates Germline Genes, Retroelements, and Immune Mediators: Implications for Facioscapulohumeral Dystrophy. Dev Cell 2012. DOI: S1534-5807(11)00523-5 [pii]10.1016/j.devce1.2011.11.013
9. Vanderplanck C, Ansseau E, Charron S, Stricwant N, Tassin A, Laoudj-Chenivesse D, Wilton S D, Coppee F, Belayew A. The FSHD atrophic myotube phenotype is caused by DUX4 expression. PLoS One 6: e26820. DOI: 10.1371/journal.pone.0026820 PONE-D-11-14845 [pii]
10. Tassin A, Laoudj-Chenivesse D, Vanderplanck C, Barro M, Charron S, Ansseau E, Chen Y W, Mercier J, Coppee F, Belayew A. DUX4 expression in FSHD muscle cells: how could such a rare protein cause a myopathy? Journal of cellular and molecular medicine 2012; 17: 76-89. DOI: 10.1111/j.1582-4934.2012.01647.x
11. Bosnakovski D, Lamb S, Simsek T, Xu Z, Belayew A, Perlingeiro R, Kyba M. DUX4c, an FSHD candidate gene, interferes with myogenic regulators and abolishes myoblast differentiation. Exp Neurol 2008; 214: 87-96. DOI: 10.1016/j.expneuro1.2008.07.022
12. Feng Q, Snider L, Jagannathan S, Tawil R, van der Maarel S M, Tapscott S J, Bradley R K. A feedback loop between nonsense-mediated decay and the retrogene DUX4 in facioscapulohumeral muscular dystrophy. Elife 2015; 4. DOI: 10.7554/eLife.04996
13. Tassin A, Laoudj-Chenivesse D, Vanderplanck C, Barro M, Charron S, Ansseau E, Chen Y W, Mercier J, Coppee F, Belayew A. DUX4 expression in FSHD muscle cells: how could such a rare protein cause a myopathy? J Cell Mot Med 2013; 17: 76-89. DOI:
14. Brouwer O F, Padberg G W, Wijmenga C, Frants R R. Facioscapulohumeral muscular dystrophy in early childhood. Arch Neurol 1994; 51: 387-394. DOI:
15. Lunt P W, Jardine P E, Koch M C, Maynard J, Osborn M, Williams M, Harper P S, Upadhyaya M. Correlation between fragment size at D4F104S1 and age at onset or at wheelchair use, with a possible generational effect, accounts for much phenotypic variation in 4q35-facioscapulohumeral muscular dystrophy (FSHD). Human molecular genetics 1995; 4: 951-958.
16. Tawil R, Forrester J, Griggs R C, Mendell J, Kissel J, McDermott M, King W, Weiffenbach B, Figlewicz D. Evidence for anticipation and association of deletion size with severity in facioscapulohumeral muscular dystrophy. The FSH-DY Group. Ann Neurol 1996; 39: 744-748.
17. Klinge L, Eagle M, Haggerty I D, Roberts C E, Straub V, Bushby K M. Severe phenotype in infantile facioscapulohumeral muscular dystrophy. Neuromuscul Disord 2006; 16: 553-558. DOI: 10.1016/j.nmd.2006.06.008
18. Ricci E, Galluzzi G, Deidda G, Cacurri S, Colantoni L, Merico B, Piazzo N, Servidei S, Vigneti E, Pasceri V, Silvestri G, Mirabella M, Mangiola F, Tonali P, Felicetti L. Progress in the molecular diagnosis of facioscapulohumeral muscular dystrophy and correlation between the number of KpnI repeats at the 4q35 locus and clinical phenotype. Ann Neurol 1999; 45: 751-757. DOI: 10.1002/1531-8249(199906)45:6<751::aid-ana9>3.0.co;2-m
19. Hoffman E P, Connor E M. Orphan drug development in muscular dystrophy: update on two large clinical trials of dystrophin rescue therapies. Discov Med 2013; 16: 233-239.
20. Mercuri E, Messina S, Pane M, Bertini E. Current methodological issues in the study of children with inherited neuromuscular disorders. Dev Med Child Neurol 2008; 50: 417-421. DOI: 10.1111/j.1469-8749.2008.02066.x
21. Califf R M. Biomarker definitions and their applications. Exp Blot Med (Maywood) 2018; 243: 213-221. DOI: 10.1177/1535370217750088
22. Geng L N, Yao Z, Snider L, Fong A P, Cech J N, Young J M, van der Maarel S M, Ruzzo W L, Gentleman R C, Tawil R, Tapscott S J. DUX4 activates germline genes, retroelements, and immune mediators: implications for facioscapulohumeral dystrophy. Dev Cell 2012; 22: 38-51. DOI: 10.1016/j.devce1.2011.11.013
23. Consortium E P. An integrated encyclopedia of DNA elements in the human genome. Nature 2012; 489: 57-74. DOI: 10.1038/nature11247
24. Davis C A, Hitz B C, Sloan C A, Chan E T, Davidson J M, Gabdank I, Hilton J A, Jain K, Baymuradov U K, Narayanan A K, Onate K C, Graham K, Miyasato S R, Dreszer T R, Strattan J S, Jolanki O, Tanaka F Y, Cherry J M. The Encyclopedia of DNA elements (ENCODE): data portal update. Nucleic Acids Res 2018; 46: D794-D801. DOI:
25. Kent W J, Sugnet C W, Furey T S, Roskin K M, Pringle T H, Zahler A M, Haussler D. The human genome browser at UCSC. Genome Res 2002; 12: 996-1006. DOI: 10.1101/gr.229102
26. Mathelier A, Fornes O, Arenillas D J, Chen C Y, Denay G, Lee J, Shi W, Shyr C, Tan G, Worsley-Hunt R, Zhang A W, Parcy F, Lenhard B, Sandelin A, Wasserman W W. JASPAR 2016: a major expansion and update of the open-access database of transcription factor binding profiles. Nucleic Acids Res 2016; 44: D110-115. DOI:
27. Wang J, Zhuang J, Iyer S, Lin X Y, Greven M C, Kim B H, Moore J, Pierce B G, Dong X, Virgil D, Birney E, Hung J H, Weng Z. Factorbook.org: a Wiki-based database for transcription factor-binding data generated by the ENCODE consortium. Nucleic Acids Res 2013; 41: D171-176. DOI: 10.1093/nar/gks1221
28. Mah J K, Feng J, Jacobs M B, Duong T, Carroll K, de Valle K, Carty C L, Morgenroth L P, Guglieri M, Ryan M M, Clemens P R, Thangarajh M, Webster R, Smith E, Connolly A M, McDonald C M, Karachunski P, Tulinius M, Harper A, Cnaan A, Chen Y W, Cooperative International Neuromuscular Research Group I. A multinational study on motor function in early-onset FSHD. Neurology 2018; 90: e1333-e1338. DOI:
29. Batra S K, Heier C R, Diaz-Calderon L, Tully C B, Fiorillo A A, van den Anker J, Conklin L S. Serum miRNAs Are Pharmacodynamic Biomarkers Associated With Therapeutic Response in Pediatric Inflammatory Bowel Disease. Inflamm Bowel Dis 2020. DOI:
30. Heier C R, Fiorillo A A, Chaisson E, Gordish-Dressman H, Hathout Y, Damsker J M, Hoffman E P, Conklin L S. Identification of Pathway-Specific Serum Biomarkers of Response to Glucocorticoid and Infliximab Treatment in Children with Inflammatory Bowel Disease. Clin Transl Gastroenterol 2016; 7: e192. DOI: 10.1038/ctg.2016.49
31. Zahm A M, Thayu M, Hand N J, Horner A, Leonard M B, Friedman J R. Circulating microRNA is a biomarker of pediatric Crohn disease. J Pediatr Gastroenterol Nutr 2011; 53: 26-33. DOI: 10.1097/MPG.0b013e31822200cc
32. Cox J, Mann M. MaxQuant enables high peptide identification rates, individualized p.p.b.-range mass accuracies and proteome-wide protein quantification. Nat Biotechnol 2008; 26: 1367-1372. DOI: 10.1038/nbt.1511
33. Fiorillo A A, Heier C R, Novak J S, Tully C B, Brown K J, Uaesoontrachoon K, Vila M C, Ngheim P P, Bello L, Kornegay J N, Angelini C, Partridge T A, Nagaraju K, Hoffman E P. TNF-alpha-Induced microRNAs Control Dystrophin Expression in Becker Muscular Dystrophy. Cell Rep 2015; 12: 1678-1690. DOI: 10.1016/j.celrep.2015.07.066
34. Fiorillo A A, Tully C B, Damsker J M, Nagaraju K, Hoffman E P, Heier C R. Muscle miRNAome shows suppression of chronic inflammatory miRNAs with both prednisone and vamorolone. Physiol Genomics 2018; 50: 735-745. DOI:
35. Small E M, O'Rourke J R, Moresi V, Sutherland L B, McAnally J, Gerard R D, Richardson J A, Olson E N. Regulation of PI3-kinase/Akt signaling by muscle-enriched microRNA-486. Proc Natl Acad Sci USA 2010; 107: 4218-4223. DOI: 10.1073/pnas.1000300107
36. Yan Y, Shi R, Yu X, Sun C, Zang W, Tian H. Identification of atrial fibrillation-associated microRNAs in left and right atria of rheumatic mitral valve disease patients. Genes Genet Syst 2019; 94: 23-34. DOI: 10.1266/ggs.17-00043
37. Sen A, Ren S, Lerchenmuller C, Sun J, Weiss N, Most P, Peppel K. MicroRNA-138 regulates hypoxia-induced endothelial cell dysfunction by targeting S100A1. PLoS One 2013; 8: e78684. DOI: 10.1371/journal.pone.0078684
38. Yu J, Lu Y, Li Y, Xiao L, Xing Y, Li Y, Wu L. Role of S100A1 in hypoxia-induced inflammatory response in cardiomyocytes via TLR4/ROS/NF-kappaB pathway. J Pharm Pharmacol 2015; 67: 1240-1250. DOI: 10.1111/jphp.12415
39. Duan Y, Zhou M, Xiao J, Wu C, Zhou L, Zhou F, Du C, Song Y. Prediction of key genes and miRNAs responsible for loss of muscle force in patients during an acute exacerbation of chronic obstructive pulmonary disease. Int J Mol Med 2016; 38: 1450-1462. DOI:
40. Yin H, He H, Shen X, Zhao J, Cao X, Han S, Cui C, Chen Y, Wei Y, Xia L, Wang Y, Li D, Zhu Q. miR-9-5p Inhibits Skeletal Muscle Satellite Cell Proliferation and Differentiation by Targeting I G F2BP3 through the IGF2-PI3K/Akt Signaling Pathway. Int J Mol Sci 2020; 21. DOI: 10.3390/ijms21051655
41. Shen J, Xing W, Liu R, Zhang Y, Xie C, Gong F. MiR-32-5p influences high glucose-induced cardiac fibroblast proliferation and phenotypic alteration by inhibiting DUSP1. BMC Mol Biol 2019; 20: 21. DOI: 10.1186/s12867-019-0135-x
42. Liu J, Xiao X, Shen Y, Chen L, Xu C, Zhao H, Wu Y, Zhang Q, Zhong J, Tang Z, Liu C, Zhao Q, Zheng Y, Cao R, Zu X. MicroRNA-32 promotes calcification in vascular smooth muscle cells: Implications as a novel marker for coronary artery calcification. PLoS One 2017; 12: e0174138. DOI: 10.1371/journal.pone.0174138
43. Lee S Y, Yang J, Park J H, Shin H K, Kim W J, Kim S Y, Lee E J, Hwang I, Lee C S, Lee J, Kim H S. The MicroRNA-92a/Sp1/MyoD Axis Regulates Hypoxic Stimulation of Myogenic Lineage Differentiation in Mouse Embryonic Stem Cells. Mol Ther 2020; 28: 142-156. DOI: 10.1016/j.ymthe.2019.08.014
44. Lazzarini R, Caffarini M, Delli Carpini G, Ciavattini A, Di Primio R, Orciani M. From 2646 to 15: differentially regulated microRNAs between progenitors from normal myometrium and leiomyoma. Am J Obstet Gynecol 2020; 222: 596 e591-596 e599. DOI:
45. Kinder T B, Heier C R, Tully C B, Van der Muelen J H, Hoffman E P, Nagaraju K, Fiorillo A A. Muscle Weakness in Myositis: MicroRNA-Mediated Dystrophin Reduction in a Myositis Mouse Model and Human Muscle Biopsies. Arthritis Rheumatol 2020; 72: 1170-1183. DOI: 10.1002/art.41215
46. Liu H L, Zhu J G, Liu Y Q, Fan Z G, Zhu C, Qian L M. Identification of the microRNA expression profile in the regenerative neonatal mouse heart by deep sequencing. Cell Biochem Biophys 2014; 70: 635-642. DOI: 10.1007/s12013-014-9967-7
47. Li J, Chan M C, Yu Y, Bei Y, Chen P, Zhou Q, Cheng L, Chen L, Ziegler O, Rowe G C, Das S, Xiao J. miR-29b contributes to multiple types of muscle atrophy. Nat Commun 2017; 8: 15201. DOI: 10.1038/ncomms15201
48. Li J, Wang L, Hua X, Tang H, Chen R, Yang T, Das S, Xiao J. CRISPR/Cas9-Mediated miR-29b Editing as a Treatment of Different Types of Muscle Atrophy in Mice. Mol Ther 2020; 28: 1359-1372. DOI: 10.1016/j.ymthe.2020.03.005
49. Wang J, Pei Y, Zhong Y, Jiang S, Shao J, Gong J. Altered serum microRNAs as novel diagnostic biomarkers for atypical coronary artery disease. PLoS One 2014; 9: e107012. DOI: 10.1371/journal.pone.0107012
50. Dmitriev P, Barat A, Polesskaya A, O'Connell M J, Robert T, Dessen P, Walsh T A, Lazar V, Turki A, Carnac G, Laoudj-Chenivesse D, Lipinski M, Vassetzky Y S. Simultaneous miRNA and mRNA transcriptome profiling of human myoblasts reveals a novel set of myogenic differentiation-associated miRNAs and their target genes. BMC Genomics 2013; 14: 265. DOI: 10.1186/1471-2164-14-265
51. Kropp J, Degerny C, Morozova N, Pontis J, Harel-Bellan A, Polesskaya A. miR-98 delays skeletal muscle differentiation by down-regulating E2F5. Biochem J 2015; 466: DOI: 10.1042/BJ20141175
52. Ghorbanmehr N, Gharbi S, Korsching E, Tavallaei M, Einollahi B, Mowla S J. miR-21-miR-141-3p, and miR-205-5p levels in urine-promising biomarkers for the identification of prostate and bladder cancer. Prostate 2019; 79: 88-95. DOI:
53. Greco S, Perfetti A, Fasanaro P, Cardani R, Capogrossi M C, Meola G, Martelli F. Deregulated microRNAs in myotonic dystrophy type 2. PLoS One 2012; 7: e39732. DOI:
54. Portilho D M, Alves M R, Kratassiouk G, Roche S, Magdinier F, de Santana E C, Polesskaya A, Harel-Bellan A, Mouly V, Savino W, Butler-Browne G, Dumonceaux J. miRNA expression in control and FSHD fetal human muscle biopsies. PLoS One 2015; e0116853. DOI: 10.1371/journal.pone.0116853
55. Perfetti A, Greco S, Cardani R, Fossati B, Cuomo G, Valaperta R, Ambrogi F, Cortese A, Botta A, Mignarri A, Santoro M, Gaetano C, Costa E, Dotti M T, Silvestri G, Massa R, Meola G, Martelli F. Validation of plasma microRNAs as biomarkers for myotonic dystrophy type 1. Sci Rep 2016; 6: 38174. DOI: 10.1038/srep38174
56. Perfetti A, Greco S, Bugiardini E, Cardani R, Gaia P, Gaetano C, Meola G, Martelli F. Plasma microRNAs as biomarkers for myotonic dystrophy type 1. Neuromuscul Disord 2014; 24: 509-515. DOI: 10.1016/j.nmd.2014.02.005
57. Sylvius N, Bonne G, Straatman K, Reddy T, Gant T W, Shackleton S. MicroRNA expression profiling in patients with lamin A/C-associated muscular dystrophy. FASEB J 2011; 25: 3966-3978. DOI: 10.1096/fj.11-182915
58. Gao Y Q, Chen X, Wang P, Lu L, Zhao W, Chen C, Chen C P, Tao T, Sun J, Zheng Y Y, Du J, Li C J, Gan Z J, Gao X, Chen H Q, Zhu M S. Regulation of DLK1 by the maternally expressed miR-379/miR-544 cluster may underlie callipyge polar overdominance inheritance. Proc Natl Acad Sci USA 2015; 112: 13627-13632. DOI:
59. Jarlborg M, Courvoisier D S, Lamacchia C, Martinez Prat L, Mahler M, Bentow C, Finckh A, Gabay C, Nissen M J, physicians of the Swiss Clinical Quality Management r. Serum calprotectin: a promising biomarker in rheumatoid arthritis and axial spondyloarthritis. Arthritis Res Ther 2020; 22: 105. DOI: 10.1186/s13075-020-02190-3
60. Metz M, Torene R, Kaiser S, Beste M T, Staubach P, Bauer A, Brehler R, Gericke J, Letzkus M, Hartmann N, Erpenbeck V J, Maurer M. Omalizumab normalizes the gene expression signature of lesional skin in patients with chronic spontaneous urticaria: A randomized, double-blind, placebo-controlled study. Allergy 2019; 74: 141-151. DOI:
61. Wang S, Song R, Wang Z, Jing Z, Wang S, Ma J. S100A8/A9 in Inflammation. Front Immunol 2018; 9: 1298. DOI: 10.3389/fimmu.2018.01298
62. Kalla R, Kennedy N A, Ventham N T, Boyapati R K, Adams A T, Nimmo E R, Visconti M R, Drummond H, Ho G T, Pattenden R J, Wilson D C, Satsangi J. Serum Calprotectin: A Novel Diagnostic and Prognostic Marker in Inflammatory Bowel Diseases. Am J Gastroenterol 2016; 111: 1796-1805. DOI: 10.1038/ajg.2016.342
63. Pass H I, Levin S M, Harbut M R, Melamed J, Chiriboga L, Donington J, Huflejt M, Carbone M, Chia D, Goodglick L, Goodman G E, Thornquist M D, Liu G, de Perrot M, Tsao M S, Goparaju C. Fibulin-3 as a blood and effusion biomarker for pleural mesothelioma. N Engl J Med 2012; 367: 1417-1427. DOI: 10.1056/NEJMoa1115050
64. Zhang X, Yin M, Zhang L J. Keratin 6, 16 and 17-Critical Barrier Alarmin Molecules in Skin Wounds and Psoriasis. Cells 2019; 8. DOI: 10.3390/ce11s8080807
65. Rojahn T B, Vorstandlechner V, Krausgruber T, Bauer W M, Alkon N, Bangert C, Thaler F M, Sadeghyar F, Fortelny N, Gernedl V, Rindler K, Elbe-Burger A, Bock C, Mildner M, Brunner P M. Single-cell transcriptomics combined with interstitial fluid proteomics defines cell type-specific immune regulation in atopic dermatitis. J Allergy Clin Immunol 2020. DOI: 10.1016/j.jaci.2020.03.041
66. Zouboulis C C, Nogueira da Costa A, Makrantonaki E, Hou X X, Almansouri D, Dudley J T, Edwards H, Readhead B, Balthasar O, Jemec G B E, Bonitsis N G, Nikolakis G, Trebing D, Zouboulis K C, Hossini A M. Alterations in innate immunity and epithelial cell differentiation are the molecular pillars of hidradenitis suppurativa. J Eur Acad Dermatol Venereol 2020; 34: 846-861. DOI: 10.1111/jdv.16147
67. Mechtcheriakova D, Wlachos A, Sobanov J, Kopp T, Reuschel R, Bornancin F, Cai R, Zemann B, Urtz N, Stingl G, Zlabinger G, Woisetschlager M, Baumruker T, Billich A. Sphingosine 1-phosphate phosphatase 2 is induced during inflammatory responses. Cell Signal 2007; 19: 748-760. DOI: 10.1016/j.cellsig.2006.09.004
68. Vetrano S, Ploplis V A, Sala E, Sandoval-Cooper M, Donahue D L, Correale C, Arena V, Spinelli A, Repici A, Malesci A, Castellino F J, Danese S. Unexpected role of anticoagulant protein C in controlling epithelial barrier integrity and intestinal inflammation. Proc Natl Acad Sci USA 2011; 108: 19830-19835. DOI:
69. Danese S, Vetrano S, Zhang L, Poplis V A, Castellino F J. The protein C pathway in tissue inflammation and injury: pathogenic role and therapeutic implications. Blood 2010; 115: 1121-1130. DOI: 10.1182/blood-2009-09-201616
70. Alquraini A, Garguilo S, D'Souza G, Zhang L X, Schmidt T A, Jay G D, Elsaid K A. The interaction of lubricin/proteoglycan 4 (PRG4) with toll-like receptors 2 and 4: an anti-inflammatory role of PRG4 in synovial fluid. Arthritis Res Ther 2015; 17: 353. DOI:
71. Kosinska M K, Ludwig T E, Liebisch G, Zhang R, Siebert H C, Wilhelm J, Kaesser U, Dettmeyer R B, Klein H, Ishaque B, Rickert M, Schmitz G, Schmidt T A, Steinmeyer J. Articular Joint Lubricants during Osteoarthritis and Rheumatoid Arthritis Display Altered Levels and Molecular Species. PLoS One 2015; 10: e0125192. DOI:
72. Vanderplanck C, Ansseau E, Charron S, Stricwant N, Tassin A, Laoudj-Chenivesse D, Wilton S D, Coppee F, Belayew A. The FSHD atrophic myotube phenotype is caused by DUX4 expression. PLoS One 2011; 6: e26820. DOI: 10.1371/journal.pone.0026820
73. Wallace L M, Garwick-Coppens S E, Tupler R, Harper S Q. RNA interference improves myopathic phenotypes in mice over-expressing FSHD region gene 1 (FRG1). Mol Ther 2011; 19: 2048-2054. DOI: 10.1038/mt.2011.118
74. Pandey S N, Cabotage J, Shi R, Dixit M, Sutherland M, Liu J, Muger S, Harper S Q, Nagaraju K, Chen Y W. Conditional over-expression of PITX1 causes skeletal muscle dystrophy in mice. Biol Open 2012; 1: 629-639. DOI: 10.1242/bio.20121305
75. Block G J, Narayanan D, Amell A M, Petek L M, Davidson K C, Bird T D, Tawil R, Moon R T, Miller D G. Wnt/beta-catenin signaling suppresses DUX4 expression and prevents apoptosis of FSHD muscle cells. Hum Mol Genet 2013; 22: 4661-4672. DOI:
76. Cacchiarelli D, Legnini I, Martone J, Cazzella V, D'Amico A, Bertini E, Bozzoni I. miRNAs as serum biomarkers for Duchenne muscular dystrophy. EMBO Mot Med 2011; 3: 258-265. DOI: 10.1002/emmm.201100133
77. Matsuzaka Y, Kishi S, Aoki Y, Komaki H, Oya Y, Takeda S, Hashido K. Three novel serum biomarkers, miR-1, miR-133a, and miR-206 for Limb-girdle muscular dystrophy, Facioscapulohumeral muscular dystrophy, and Becker muscular dystrophy. Environ Health Prev Med 2014; 19: 452-458. DOI: 10.1007/s12199-014-0405-7
78. Statland J, Donlin-Smith C M, Tapscott S J, van der Maarel S, Tawil R. Multiplex Screen of Serum Biomarkers in Facioscapulohumeral Muscular Dystrophy. J Neuromuscul Dis 2014; 1: 181-190. DOI: 10.3233/JND-140034
79. Petek L M, Rickard A M, Budech C, Poliachik S L, Shaw D, Ferguson M R, Tawil R, Friedman S D, Miller D G. A cross sectional study of two independent cohorts identifies serum biomarkers for facioscapulohumeral muscular dystrophy (FSHD). Neuromuscul Disord 2016; 26: 405-413. DOI: 10.1016/j.nmd.2016.04.012
80. Konikoff M R, Denson L A. Role of fecal calprotectin as a biomarker of intestinal inflammation in inflammatory bowel disease. Inflamm Bowel Dis 2006; 12: 524-534. DOI: 10.1097/00054725-200606000-00013
81. Foell D, Wulffraat N, Wedderburn L R, Wittkowski H, Frosch M, Gerss J, Stanevicha V, Mihaylova D, Ferriani V, Tsakalidou F K, Foeldvari I, Cuttica R, Gonzalez B, Ravelli A, Khubchandani R, Oliveira S, Armbrust W, Garay S, Vojinovic J, Norambuena X, Gamir M L, Garcia-Consuegra J, Lepore L, Susic G, Corona F, Dolezalova P, Pistorio A, Martini A, Ruperto N, Roth J, Paediatric Rheumatology International Trials 0. Methotrexate withdrawal at 6 vs 12 months in juvenile idiopathic arthritis in remission: a randomized clinical trial. AMA 2010; 303: 1266-1273. DOI: 10.1001/jama.2010.375
82. Vogl T, Eisenblatter M, Voller T, Zenker S, Hermann S, van Lent P, Faust A, Geyer C, Petersen B, Roebrock K, Schafers M, Bremer C, Roth J. Alarmin S100A8/S100A9 as a biomarker for molecular imaging of local inflammatory activity. Nat Commun 2014; 5: 4593. DOI: 10.1038/ncomms5593
83. Nistala K, Varsani H, Wittkowski H, Vogl T, Krol P, Shah V, Mamchaoui K, Brogan P A, Roth J, Wedderburn L R. Myeloid related protein induces muscle derived inflammatory mediators in juvenile dermatomyositis. Arthritis Res Ther 2013; 15: R131. DOI:

Table 51 discloses additional information about the biomarkers and their use of diagnosing FSHD. It is described by the following pages and forms an integral part of the specification.

TABLE S1
Transcription Factor ChIP-seq Clusters (161 factors) from ENCODE with ENCODE 3 Motifs (# of bound promoter or enhancer sites for each miRNA gene)
3 databases: UCSC Genes, RefSeq All, UCSC RefSeq

| SUMMARY | ARID3A | ATF3 | BCL3 | CEBPB | CREB1 | EGR1 | ETS1 | ETV6 | EZH2 |
|---|---|---|---|---|---|---|---|---|---|
| Total # of binding | 73 | 93 | 56 | 115 | 61 | 104 | 70 | 28 | 46 |
| sites in all miRNAs | | | | | | | | | |

promoter = within 2 kb
enhancer = within 10 kb

| | | | | | | | | | | | | | |
|---|---|---|---|---|---|---|---|---|---|---|---|---|---|
| microRNA | locus | Home Gene | Promoter/Enhance | Notes | ARID3A | ATF3 | BCL3 | CEBPB | CREB1 | EGR1 | ETS1 | ETV6 | EZH2 |
| hsa-miR-32 | 9q31.3 | TMEM245 | TMEM245 promoter | | 1 | 1 | | | 1 | 2 | 1 | | |
| | | | TMEM245 enhancer | | 5 | 4 | | 5 | 2 | 3 | 1 | | |
| | | | TMEM245 promoter | low H3K4me3 signal | 1 | 1 | | 1 | | | | | |
| | | | TMEM245 enhancer | | 5 | 4 | 1 | 4 | 1 | | | 2 | |
| | | | TMEM245 promoter | low H3K4me3 signal | | | | 1 | | | | | |
| | | | TMEM245 enhancer | | 1 | 1 | 1 | 2 | | | | | |
| | | | miR-32 promoter | low H3K4me3 signal | | | | | | | | | |
| | | | miR-32 enhancer | | 1 | 2 | 1 | 1 | | 1 | 1 | | |
| hsa-miR-138-1 | 3p21.3 | independent | miR-138-1 promoter | low H3K4me3 signal | | | | | | | | | |
| | | | miR-138-1 enhancer | | | 1 | 2 | | 1 | | | | |
| hsa-miR-103-1 | 5q34 | PANK3 | PANK3 promoter | | 1 | 1 | | | 1 | 1 | 1 | | |
| | | | PANK3 enhancer | | 1 | 2 | | 3 | 3 | 1 | 1 | | |
| | | | PANK3 promoter | | 1 | 1 | | | 1 | 1 | 1 | | |
| | | | PANK3 enhancer | | 1 | 2 | | 3 | 3 | 1 | 1 | | |
| | | | miR-103-1 promoter | low H3K4me3 signal | | | | | | | | | |
| | | | miR-103-1 enhancer | | | | | 1 | | | | | |
| hsa-miR-505 | Xq27.1 | ATP11C | ATP11C promoter | | | | | | | 1 | 1 | | |
| | | | ATP11C enhancer | | 1 | | | 1 | | 2 | 1 | | |
| | | | miR-505 promoter | low H3K4me3 signal | | | | 1 | | | | | |
| | | | miR-505 enhancer | | 1 | | | 1 | | 2 | 1 | | |
| hsa-miR-146b | 10q24.3 | independent | miR-146b promoter | | | 2 | | 1 | 1 | 2 | | | 1 |
| | | | miR-146b enhancer | | | 4 | 1 | 3 | 2 | 4 | 1 | | 1 |
| hsa-miR-29b-1 | 7q32.3 | LOC646329 | LOC646329 promoter | | 1 | 1 | 1 | 2 | | 1 | 1 | 1 | |
| | | | LOC646329 enhancer | | 6 | 9 | 5 | 10 | 2 | 3 | 6 | 1 | |
| | | | miR-29b-1 promoter | low H3K4me3 signal | | | | | | | | | |
| | | | miR-29b-1 enhancer | | | 4 | 4 | 4 | | 2 | 3 | | |
| hsa-miR-486 | 8p11.2 | NKX6-3 and | NKX6-3 promoter | low H3K4me3 signal | | | | | | | | | |
| | | ANK1 | | | | | | | | | | | |
| | | | NKX6-3 enhancer | | 1 | 2 | | | | 3 | 1 | | |
| | | | ANK1 promoter | | | | | | | | | | |
| | | | ANK1 enhancer | | | 1 | 1 | | 1 | | | | |
| | | | ANK1 promoter | low H3K4me3 signal | | | | | | | | | |
| | | | ANK1 enhancer | | 3 | 2 | 2 | 2 | | | 1 | 1 | 1 |
| | | | ANK1 promoter | | | 1 | | 2 | | 3 | 2 | | 1 |
| | | | ANK1 enhancer | | 1 | 4 | | 2 | | 6 | 3 | | 1 |
| | | | ANK1 promoter | | | | | | | | | | 2 |
| | | | ANK1 enhancer | | | 1 | | 1 | 2 | | | | 4 |
| | | | miR-486 promoter | low H3K4me3 signal | | | | | | | | | |
| | | | miR-486 enhancer | | | 1 | 1 | | | 1 | | | 1 |
| hsa-miR-34a | 1p36.23 | mir-34aHG | mir-34aHG Promoter | | 1 | | 1 | | | | | | 1 |
| | | | mir-34aHG Enhancer | | 2 | | 2 | 1 | | | | | 1 |
| | | | miR-34a promoter | low H3K4me3 signal | | 1 | | | | | | | |
| | | | miR-34a enhancer | | | 3 | | 3 | 1 | 1 | | | |
| hsa-miR-141 | 12p13.3 | miR-200C HG | miR-200C HG promoter | | | 1 | 1 | 1 | 1 | 2 | 2 | 1 | |
| | | | miR-141 promoter | | | | 1 | 1 | | 1 | 2 | | |
| | | | miR-141/miR-200C HG | miRNAs share | 3 | 2 | 5 | 3 | 2 | 6 | 6 | 4 | |
| | | | enhancer | regulatory elements | | | | | | | | | |
| hsa-miR-98 | Xp11.2 | HUWE1 | HUWE1 promoter | low H3K4me3 signal | | | | 1 | | | | 1 | |
| | | | HUWE1 enhancer | | | | | 1 | | | | 1 | |
| | | | HUWE1 promoter | | | | | | | | | | |
| | | | HUWE1 enhancer | | 1 | | 1 | | 1 | 3 | | | |
| | | | miR-98 promoter | low H3K4me3 signal | | | | | | | | | |
| | | | miR-98 enhancer | | | | | | | | | | |
| hsa-miR-576 | 4q25 | SEC24B | SEC24B promoter | | 2 | | | 1 | 1 | 1 | 1 | | |
| | | | SEC24B enhancer | | 3 | 2 | | 3 | 1 | 3 | 1 | | |
| | | | miR-576 promoter | low H3K4me3 signal | | | | | | | | | |
| | | | miR-576 enhancer | | | | | | | | | | |
| hsa-miR-9-1 | 1q22 | C1orf61 | C1orf61 Promoter | low H3K4me3 signal | | | | | 1 | 2 | | | 2 |
| | | | C1orf61 Enhancer | | | 2 | 1 | 1 | 3 | 3 | | 1 | 5 |
| | | | C1orf61 Promoter | | | 2 | | 1 | 1 | 1 | | | 1 |
| | | | C1orf61 Enhancer | | | 2 | 1 | 1 | 2 | 4 | | | 6 |
| | | | miR-9-1 promoter | | | 1 | | | 1 | 1 | | | 1 |
| | | | miR-9-1 enhancer | | | 2 | 1 | 1 | 2 | 4 | | | 5 |
| hsa-miR-142 | 17q22 | independent | miR-142 promoter | | 1 | 2 | 3 | | | 3 | 3 | 3 | |
| | | | miR-142 enhancer | | 4 | 5 | 9 | 6 | 3 | 9 | 11 | 11 | 3 |
| hsa-miR-92a-1 | 13q31.3 | miR-17HG/ | miR-17 promoter | | 1 | | | | | | 1 | | 1 |
| | | miR-19B1 | | | | | | | | | | | |
| | | | miR-17 HG enhancer | | 5 | 1 | 2 | 3 | 1 | 2 | 4 | | 1 |
| | | | miR-19B1 promoter | | 2 | | 1 | | | 1 | 2 | | |
| | | | miR-92a-1 promoter | | | | | | | | 1 | | |
| | | | miR-19B1/miR-92a-1 | share regulatory | 4 | | 2 | 1 | | 2 | 2 | | 1 |
| | | | | elements | | | | | | | | | |
| hsa-miR-502 | Xp11.2 | CLCN5 | CLCN5 promoter | | | | | | | | | | |
| | | | CLCN5 enhancer | | | | | 1 | | 1 | | | |
| | | | miR-502 promoter | low H3K4me3 signal | | | | | | | | | |
| | | | miR-502 promoter | | | | | | | | | | |
| hsa-miR-140 | 16q22.1 | WWP2 | WWP2 Promoter | | 1 | 1 | | | | 1 | 1 | | |
| | | | WWP2 Enhancer | | 2 | 3 | | 2 | 1 | 4 | 2 | | |
| | | | WWP2 Promoter | low H3K4me3 signal | | | | | | | | | |
| | | | WWP2 Enhancer | | 2 | | | 3 | | | | | |
| | | | WWP2 Promoter | low H3K4me3 signal | | | | | | | | | |
| | | | WWP2 Enhancer | | | | | 1 | | | | | |
| | | | WWP2 Promoter | High H3K4me3 | | | | | | | | | |
| | | | WWP2 Enhancer | | | | | 1 | | | | | |
| | | | miR-140 promoter | low H3K4me3 signal | | | | | | | | | |
| | | | miR-140 enhancer | | | | | 1 | | | 1 | | |
| hsa-miR-100 | 11q24.1 | miR-100HG | miR-100HG Promoter | | | 1 | | | 2 | | | | |
| | | | miR-100HG Enhancer | | | 4 | | 6 | 2 | | | | |
| | | | miR-100HG Promoter | | | 1 | | 2 | | | | | |
| | | | miR-100HG Enhancer | | 3 | 1 | 4 | | 1 | | | | |
| | | | miR-100HG Promoter | low H3K4me3 signal | | | | | | | | | 1 |
| | | | miR-100HG Enhancer | | | | | 1 | | 1 | | | 2 |
| | | | miR-100HG Promoter | | | 1 | | | 1 | 1 | | | |
| | | | miR-100HG Enhancer | | | 1 | | | 1 | 1 | | | 1 |
| | | | miR-100HG Promoter | low H3K4me3 signal | | | | 1 | | | | | |
| | | | miR-100HG Enhancer | | | | | 1 | | | | | |
| | | | miR-100HG Promoter | | | | | | | | | | |
| | | | miR-100HG Promoter | | | | | | | | | | |
| | | | miR-100HG Enhancer | | 1 | | | 4 | | | | | |
| | | | miR-100 promoter | | | | | 1 | | | | | |
| | | | miR-100 enhancer | | 1 | | | 3 | | | | | |
| hsa-miR-329-1 | 14q32.3 | independent | miR-329-1 promoter | low H3K4me3 signal | | | | | 2 | | | | |
| | | | miR-329-1 enhancer | low histone mark | | | | | 3 | | | | |
| | | | | signals | | | | | | | | | |
| hsa-miR-454 | 17q22 | SKA2 | SKA2 promoter | | 1 | 1 | | 1 | 1 | 2 | | | 1 |
| | | | SKA2 enhancer | | 1 | 1 | 1 | 4 | 3 | 4 | 1 | 1 | 1 |
| | | | miR-454 promoter | | | | | 1 | | | | | |
| | | | miR-454 enhancer | | | | | 1 | 2 | | 1 | | |

| | | | | SUMMARY | FOS | HDAC2 | IRF1 | JUN | JUNB | KAT2B | KDM5B |
|---|---|---|---|---|---|---|---|---|---|---|---|
| | | | | Total # of binding | 147 | 96 | 53 | 69 | 41 | 10 | 53 |
| | | | | sites in all miRNAs | | | | | | | |
| promoter = within 2 kb | | | | | | | | | | | |
| enhancer = within 10 kb | | | | | | | | | | | |
| microRNA | locus | Home Gene | Promoter/Enhance | Notes | FOS | HDAC2 | IRF1 | JUN | JUNB | KAT2B | KDM5B |
| hsa-miR-32 | 9q31.3 | TMEM245 | TMEM245 promoter | | 1 | 1 | 1 | 1 | 1 | | 1 |
| | | | TMEM245 enhancer | | 5 | 5 | 5 | 3 | 4 | | 1 |
| | | | TMEM245 promoter | low H3K4me3 signal | 1 | 1 | 1 | | | | |
| | | | TMEM245 enhancer | | 6 | 4 | 4 | 3 | 5 | | |
| | | | TMEM245 promoter | low H3K4me3 signal | 1 | | | | | | |
| | | | TMEM245 enhancer | | 2 | 1 | | | 2 | | |
| | | | miR-32 promoter | low H3K4me3 signal | | | | | | | |
| | | | miR-32 enhancer | | 4 | 1 | | 1 | | | |
| hsa-miR-138-1 | 3p21.3 | independent | miR-138-1 promoter | low H3K4me3 signal | 1 | | | | | 1 | |
| | | | miR-138-1 enhancer | | 3 | | | 1 | 1 | 1 | |
| hsa-miR-103-1 | 5q34 | PANK3 | PANK3 promoter | | 1 | | 2 | 1 | | | 1 |
| | | | PANK3 enhancer | | 3 | 1 | 2 | 2 | 2 | | 2 |
| | | | PANK3 promoter | | 1 | | 2 | 1 | | | 2 |
| | | | PANK3 enhancer | | 3 | 1 | 2 | 2 | 2 | | 2 |
| | | | miR-103-1 promoter | low H3K4me3 signal | | | | | | | |
| | | | miR-103-1 enhancer | | 2 | | | | | | |
| hsa-miR-505 | Xq27.1 | ATP11C | ATP11C promoter | | | | 1 | | | | 1 |
| | | | ATP11C enhancer | | 1 | | 1 | | | | 2 |
| | | | miR-505 promoter | low H3K4me3 signal | | | | | | | |
| | | | miR-505 enhancer | | | | 1 | | | | 2 |
| hsa-miR-146b | 10q24.3 | independent | miR-146b promoter | | 1 | 1 | 1 | | | | 1 |
| | | | miR-146b enhancer | | 3 | 3 | 2 | 1 | | | 3 |
| hsa-miR-29b-1 | 7q32.3 | LOC646329 | LOC646329 promoter | | 3 | 1 | 1 | 3 | 1 | | |
| | | | LOC646329 enhancer | | 9 | 5 | 1 | 8 | 1 | | |
| | | | miR-29b-1 promoter | low H3K4me3 signal | | | | | | | |
| | | | miR-29b-1 enhancer | | 3 | 1 | | 1 | | | |
| hsa-miR-486 | 8p11.2 | NKX6-3 and | NKX6-3 promoter | low H3K4me3 signal | | | | | | | |
| | | ANK1 | | | | | | | | | |
| | | | NKX6-3 enhancer | | 2 | 3 | 1 | 2 | 1 | | 1 |
| | | | ANK1 promoter | | | | | | | | |
| | | | ANK1 enhancer | | 2 | | | 1 | | | |
| | | | ANK1 promoter | low H3K4me3 signal | | | | | | | |
| | | | ANK1 enhancer | | 2 | | | 1 | 1 | | |
| | | | ANK1 promoter | | 2 | 2 | 1 | 2 | 1 | | 2 |
| | | | ANK1 enhancer | | 5 | 7 | 2 | 5 | 2 | | 3 |
| | | | ANK1 promoter | | | | | | | | |
| | | | ANK1 enhancer | | | 2 | | | | | |
| | | | miR-486 promoter | low H3K4me3 signal | | | | | | | |
| | | | miR-486 enhancer | | 2 | | | 1 | | | |
| hsa-miR-34a | 1p36.23 | mir-34aHG | mir-34aHG Promoter | | | | | | | | |
| | | | mir-34aHG Enhancer | | 1 | 1 | | | | 1 | |
| | | | miR-34a promoter | low H3K4me3 signal | | | | | | | |
| | | | miR-34a enhancer | | 1 | | | 1 | 1 | | |
| hsa-miR-141 | 12p13.3 | miR-200C HG | miR-200C HG promoter | | 2 | 2 | | 1 | 1 | | 1 |
| | | | miR-141 promoter | | 1 | 1 | | 1 | | | 1 |
| | | | miR-141/miR-200C HG | miRNAs share | 3 | 6 | 1 | 3 | 3 | | 2 |
| | | | enhancer | regulatory elements | | | | | | | |
| hsa-miR-98 | Xp11.2 | HUWE1 | HUWE1 promoter | low H3K4me3 signal | 1 | | | | | | |
| | | | HUWE1 enhancer | | 2 | | | | 2 | | |
| | | | HUWE1 promoter | | | | | | | | |
| | | | HUWE1 enhancer | | 1 | | 1 | 1 | | | |
| | | | miR-98 promoter | low H3K4me3 signal | | | | | | | |
| | | | miR-98 enhancer | | 1 | | | | | | |
| hsa-miR-576 | 4q25 | SEC24B | SEC24B promoter | | 1 | 1 | 1 | 1 | | 1 | 1 |
| | | | SEC24B enhancer | | 6 | 1 | 1 | 1 | 1 | 1 | 1 |
| | | | miR-576 promoter | low H3K4me3 signal | | | | | | | |
| | | | miR-576 enhancer | | | | | | | | |
| hsa-miR-9-1 | 1q22 | C1orf61 | C1orf61 Promoter | low H3K4me3 signal | | | | | | | |
| | | | C1orf61 Enhancer | | 1 | 1 | 1 | 2 | | | 2 |
| | | | C1orf61 Promoter | | 1 | 1 | 1 | 1 | | | 1 |
| | | | C1orf61 Enhancer | | 1 | 2 | 1 | 2 | | | 2 |
| | | | miR-9-1 promoter | | | | 1 | 2 | | | 2 |
| | | | miR-9-1 enhancer | | 1 | 2 | 1 | 2 | | | 2 |
| hsa-miR-142 | 17q22 | independent | miR-142 promoter | | | 2 | 2 | 2 | 1 | | 2 |
| | | | miR-142 enhancer | | 2 | 8 | 7 | 6 | 4 | | 6 |
| hsa-miR-92a-1 | 13q31.3 | miR-17HG/ | miR-17 promoter | | | 1 | | | 1 | | |
| | | miR-19B1 | | | | | | | | | |
| | | | miR-17 HG enhancer | | 1 | 3 | | | 1 | | |
| | | | miR-19B1 promoter | | | 1 | | | | | |
| | | | miR-92a-1 promoter | | | 1 | | | | | |
| | | | miR-19B1/miR-92a-1 | share regulatory | 1 | 2 | | | 1 | | |
| | | | | elements | | | | | | | |
| hsa-miR-502 | Xp11.2 | CLCN5 | CLCN5 promoter | | | 2 | | | | | |
| | | | CLCN5 enhancer | | | 3 | | | | | |
| | | | miR-502 promoter | low H3K4me3 signal | 1 | | | | | | |
| | | | miR-502 promoter | | 1 | | | | | | |
| hsa-miR-140 | 16q22.1 | WWP2 | WWP2 Promoter | | 1 | 1 | | | | | 1 |
| | | | WWP2 Enhancer | | 1 | 4 | 1 | 1 | | | 2 |
| | | | WWP2 Promoter | low H3K4me3 signal | | | | | | | |
| | | | WWP2 Enhancer | | 2 | 4 | | | | | |
| | | | WWP2 Promoter | low H3K4me3 signal | | | | | | | |
| | | | WWP2 Enhancer | | 1 | | | | | | |
| | | | WWP2 Promoter | High H3K4me3 | | | | | | | |
| | | | WWP2 Enhancer | | 1 | | | | | | |
| | | | miR-140 promoter | low H3K4me3 signal | | | | | | | |
| | | | miR-140 enhancer | | | | | | | | |
| hsa-miR-100 | 11q24.1 | miR-100HG | miR-100HG Promoter | | 1 | | | | | | |
| | | | miR-100HG Enhancer | | 8 | | | | 1 | | |
| | | | miR-100HG Promoter | | 3 | | | | | | |
| | | | miR-100HG Enhancer | | 9 | | | | | | |
| | | | miR-100HG Promoter | low H3K4me3 signal | | | | | | | |
| | | | miR-100HG Enhancer | | | | | | | | |
| | | | miR-100HG Promoter | | 1 | | | | | | |
| | | | miR-100HG Enhancer | | 2 | | | | | | |
| | | | miR-100HG Promoter | low H3K4me3 signal | 1 | | | | | | |
| | | | miR-100HG Enhancer | | 3 | | | | | | |
| | | | miR-100HG Promoter | | 1 | | | | | | |
| | | | miR-100HG Promoter | | 1 | | | | | | |
| | | | miR-100HG Enhancer | | 5 | 1 | | | | | |
| | | | miR-100 promoter | | | | | | | | |
| | | | miR-100 enhancer | | 3 | 1 | | | | | |
| hsa-miR-329-1 | 14q32.3 | independent | miR-329-1 promoter | low H3K4me3 signal | | | | | | | |
| | | | miR-329-1 enhancer | low histone mark | | | | | | | |
| | | | | signals | | | | | | | |
| hsa-miR-454 | 17q22 | SKA2 | SKA2 promoter | | 1 | 1 | 1 | 1 | | | 1 |
| | | | SKA2 enhancer | | 3 | 3 | 2 | 1 | | 1 | 2 |
| | | | miR-454 promoter | | | | | | | | |
| | | | miR-454 enhancer | | | | | | | | |

| | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
|---|---|---|---|---|---|---|---|---|---|---|---|---|---|---|---|---|
| | 30 | 127 | 41 | 2 | 10 | 66 | 6 | 2 | 49 | 52 | 42 | 35 | 12 | 4 | 81 | 23 |
| microRNA | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
| hsa-miR-32 | 1 | 2 | | | | 2 | | | 1 | 1 | 1 | 1 | | | 1 | 1 |
| | 1 | 5 | 1 | | | 2 | | | 2 | 1 | 1 | 3 | | | 4 | 1 |
| | | 3 | 1 | | | | | | 1 | | 1 | 2 | | | 4 | |
| | | | | | | | | | | | 1 | | | | 1 | |
| | | | | | | | | | 1 | | | | | | 2 | |
| hsa-miR-138-1 | | 1 | | | | | | | | | | | | | | |
| | | 2 | | | | | | | | | | | | | | |
| hsa-miR-103-1 | 1 | 1 | 1 | | | | | | | 1 | 1 | 1 | | | 1 | 1 |
| | 1 | 2 | 1 | | 1 | 1 | | | 1 | 1 | 3 | 1 | 1 | | 2 | 1 |
| | 1 | 1 | 1 | | 1 | 1 | | | | 1 | 1 | 1 | 1 | | 1 | 1 |
| | 1 | 2 | 1 | | 1 | 1 | | | 1 | 1 | 3 | 1 | 1 | | 2 | 1 |
| hsa-miR-505 | 1 | 1 | | | | 1 | | | | 1 | | | | | 1 | 1 |
| | 1 | 2 | | | | 1 | | | 2 | 1 | | | | | 1 | |
| | 1 | 2 | | | | 1 | | | 2 | 1 | | | | | 1 | 1 |

| | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
|---|---|---|---|---|---|---|---|---|---|---|---|---|---|---|---|---|
| | 11 | 21 | 7 | 39 | 53 | 18 | 3 | 164 | 16 | 30 | 3 | 49 | 45 | 51 | 1 | 7 |
| microRNA | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
| hsa-miR-32 | | 1 | | | 1 | | | 1 | | 1 | 1 | 2 | 1 | 1 | | |
| | | 1 | 1 | | 1 | 1 | | 6 | | 1 | 1 | 3 | 1 | 1 | | |
| | | 1 | | | | | | 6 | | | | | | 1 | | |
| | | | | | | | | 1 | | | | | | 1 | | |
| | | | | | | | | 1 | 1 | | | | | | | |
| hsa-miR-138-1 | | | | | | | | 1 | | | | | | | | |
| | | | | | | | | 1 | | | | | | | | |
| hsa-miR-103-1 | | | | | | | | 1 | | | | | 1 | 1 | | |
| | | | | | | 1 | | 4 | | | | 1 | 1 | 1 | | |
| | | | | | | 1 | | 2 | | | | | 1 | 1 | | |
| | | | | | | 1 | | 4 | | | | 1 | 1 | 1 | | |
| | | | | | | | | 1 | 1 | | | | | | | |
| hsa-miR-505 | | | | | | | | 1 | | | | | | | | |
| | | | | | | 1 | | 3 | | 1 | | | | 2 | | |
| | | | | | | 1 | | 3 | | 1 | | | | 2 | | |

| SUMMARY | ARID3A | ATF3 | BCL3 | CEBPB | CREB1 | EGR1 | ETS1 | ETV6 | EZH2 |
|---|---|---|---|---|---|---|---|---|---|
| Total # of binding | 73 | 93 | 56 | 115 | 61 | 104 | 70 | 28 | 46 |
| sites in all miRNAs | | | | | | | | | |

promoter = within 2 kb
enhancer = within 10 kb

| microRNA | locus | Home Gene | Promoter/Enhance | Notes | ARID3A | ATF3 | BCL3 | CEBPB | CREB1 | EGR1 | ETS1 | ETV6 | EZH2 |
|---|---|---|---|---|---|---|---|---|---|---|---|---|---|
| hsa-miR-32 | 9q31.3 | TMEM245 | TMEM245 promoter | | 1 | 1 | | | 1 | 2 | 1 | | |
| | | | TMEM245 enhancer | | 5 | 4 | | 5 | 2 | 3 | 1 | | |
| | | | TMEM245 promoter | low H3K4me3 signal | 1 | 1 | | 1 | | | | | |
| | | | TMEM245 enhancer | | 5 | 4 | 1 | 4 | 1 | | | 2 | |
| | | | TMEM245 promoter | low H3K4me3 signal | | | | 1 | | | | | |
| | | | TMEM245 enhancer | | 1 | 1 | 1 | 2 | | | | | |
| | | | miR-32 promoter | low H3K4me3 signal | | | | | | | | | |
| | | | miR-32 enhancer | | 1 | 2 | 1 | 1 | | 1 | 1 | | |
| hsa-miR-138-1 | 3p21.3 | independent | miR-138-1 promoter | low H3K4me3 signal | | | | | | | | | |
| | | | miR-138-1 enhancer | | | 1 | 2 | | 1 | | | | |
| hsa-miR-103-1 | 5q34 | PANK3 | PANK3 promoter | | 1 | 1 | | | 1 | 1 | 1 | | |
| | | | PANK3 enhancer | | 1 | 2 | | 3 | 3 | 1 | 1 | | |
| | | | PANK3 promoter | | 1 | 1 | | | 1 | 1 | 1 | | |
| | | | PANK3 enhancer | | 1 | 2 | | 3 | 3 | 1 | 1 | | |
| | | | miR-103-1 promoter | low H3K4me3 signal | | | | | | | | | |
| | | | miR-103-1 enhancer | | | | | 1 | | | | | |
| hsa-miR-505 | Xq27.1 | ATP11C | ATP11C promoter | | | | | | | 1 | 1 | | |
| | | | ATP11C enhancer | | 1 | | | 1 | | 2 | 1 | | |
| | | | miR-505 promoter | low H3K4me3 signal | | | | 1 | | | | | |
| | | | miR-505 enhancer | | 1 | | | 1 | | 2 | 1 | | |
| hsa-miR-146b | 10q24.3 | independent | miR-146b promoter | | | 2 | | 1 | 1 | 2 | | | 1 |
| | | | miR-146b enhancer | | | 4 | 1 | 3 | 2 | 4 | 1 | | 1 |
| hsa-miR-29b-1 | 7q32.3 | LOC646329 | LOC646329 promoter | | 1 | 1 | 1 | 2 | | 1 | 1 | 1 | |
| | | | LOC646329 enhancer | | 6 | 9 | 5 | 10 | 2 | 3 | 6 | 1 | |
| | | | miR-29b-1 promoter | low H3K4me3 signal | | | | | | | | | |
| | | | miR-29b-1 enhancer | | | 4 | 4 | 4 | | 2 | 3 | | |
| hsa-miR-486 | 8p11.2 | NKX6-3 and | NKX6-3 promoter | low H3K4me3 signal | | | | | | | | | |
| | | ANK1 | | | | | | | | | | | |
| | | | NKX6-3 enhancer | | 1 | 2 | | | | 3 | 1 | | |
| | | | ANK1 promoter | | | | | | | | | | |
| | | | ANK1 enhancer | | | 1 | 1 | | 1 | | | | |
| | | | ANK1 promoter | low H3K4me3 signal | | | | | | | | | |
| | | | ANK1 enhancer | | 3 | 2 | 2 | 2 | | | 1 | 1 | 1 |
| | | | ANK1 promoter | | | 1 | | 2 | | 3 | 2 | | 1 |
| | | | ANK1 enhancer | | 1 | 4 | | 2 | | 6 | 3 | | 1 |
| | | | ANK1 promoter | | | | | | | | | | 2 |
| | | | ANK1 enhancer | | | 1 | | 1 | 2 | | | | 4 |
| | | | miR-486 promoter | low H3K4me3 signal | | | | | | | | | |
| | | | miR-486 enhancer | | | 1 | 1 | | | 1 | | | 1 |
| hsa-miR-34a | 1p36.23 | mir-34aHG | mir-34aHG Promoter | | 1 | | 1 | | | | | | 1 |
| | | | mir-34aHG Enhancer | | 2 | | 2 | 1 | | | | | 1 |
| | | | miR-34a promoter | low H3K4me3 signal | | 1 | | | | | | | |
| | | | miR-34a enhancer | | | 3 | | 3 | 1 | 1 | | | |
| hsa-miR-141 | 12p13.3 | miR-200C HG | miR-200C HG promoter | | | 1 | 1 | 1 | 1 | 2 | 2 | 1 | |
| | | | miR-141 promoter | | | | 1 | 1 | | 1 | 2 | | |
| | | | miR-141/miR-200C HG | miRNAs share | 3 | 2 | 5 | 3 | 2 | 6 | 6 | 4 | |
| | | | enhancer | regulatory elements | | | | | | | | | |
| hsa-miR-98 | Xp11.2 | HUWE1 | HUWE1 promoter | low H3K4me3 signal | | | | 1 | | | | 1 | |
| | | | HUWE1 enhancer | | | | | 1 | | | | 1 | |
| | | | HUWE1 promoter | | | | | | | | | | |
| | | | HUWE1 enhancer | | 1 | | 1 | | 1 | 3 | | | |
| | | | miR-98 promoter | low H3K4me3 signal | | | | | | | | | |
| | | | miR-98 enhancer | | | | | | | | | | |
| hsa-miR-576 | 4q25 | SEC24B | SEC24B promoter | | 2 | | | 1 | 1 | 1 | 1 | | |
| | | | SEC24B enhancer | | 3 | 2 | | 3 | 1 | 3 | 1 | | |
| | | | miR-576 promoter | low H3K4me3 signal | | | | | | | | | |
| | | | miR-576 enhancer | | | | | | | | | | |
| hsa-miR-9-1 | 1q22 | C1orf61 | C1orf61 Promoter | low H3K4me3 signal | | | | | 1 | 2 | | | 2 |
| | | | C1orf61 Enhancer | | | 2 | 1 | 1 | 3 | 3 | | 1 | 5 |
| | | | C1orf61 Promoter | | | 2 | | 1 | 1 | 1 | | | 1 |
| | | | C1orf61 Enhancer | | | 2 | 1 | 1 | 2 | 4 | | | 6 |
| | | | miR-9-1 promoter | | | 1 | | | 1 | 1 | | | 1 |
| | | | miR-9-1 enhancer | | | 2 | 1 | 1 | 2 | 4 | | | 5 |
| hsa-miR-142 | 17q22 | independent | miR-142 promoter | | 1 | 2 | 3 | | | 3 | 3 | 3 | |
| | | | miR-142 enhancer | | 4 | 5 | 9 | 6 | 3 | 9 | 11 | 11 | 3 |
| hsa-miR-92a-1 | 13q31.3 | miR-17HG/ | miR-17 promoter | | 1 | | | | | | 1 | | 1 |
| | | miR-19B1 | | | | | | | | | | | |
| | | | miR-17 HG enhancer | | 5 | 1 | 2 | 3 | 1 | 2 | 4 | | 1 |
| | | | miR-19B1 promoter | | 2 | | 1 | | | 1 | 2 | | |
| | | | miR-92a-1 promoter | | | | | | | | 1 | | |
| | | | miR-19B1/miR-92a-1 | share regulatory | 4 | | 2 | 1 | | 2 | 2 | | 1 |
| | | | | elements | | | | | | | | | |
| hsa-miR-502 | Xp11.2 | CLCN5 | CLCN5 promoter | | | | | | | | | | |
| | | | CLCN5 enhancer | | | | | 1 | | 1 | | | |
| | | | miR-502 promoter | low H3K4me3 signal | | | | | | | | | |
| | | | miR-502 promoter | | | | | | | | | | |
| hsa-miR-140 | 16q22.1 | WWP2 | WWP2 Promoter | | 1 | 1 | | | | 1 | 1 | | |
| | | | WWP2 Enhancer | | 2 | 3 | | 2 | 1 | 4 | 2 | | |
| | | | WWP2 Promoter | low H3K4me3 signal | | | | | | | | | |
| | | | WWP2 Enhancer | | 2 | | | 3 | | | | | |
| | | | WWP2 Promoter | low H3K4me3 signal | | | | | | | | | |
| | | | WWP2 Enhancer | | | | | 1 | | | | | |
| | | | WWP2 Promoter | High H3K4me3 | | | | | | | | | |
| | | | WWP2 Enhancer | | | | | 1 | | | | | |
| | | | miR-140 promoter | low H3K4me3 signal | | | | | | | | | |
| | | | miR-140 enhancer | | | | | 1 | | | 1 | | |
| hsa-miR-100 | 11q24.1 | miR-100HG | miR-100HG Promoter | | | 1 | | | 2 | | | | |
| | | | miR-100HG Enhancer | | | 4 | | 6 | 2 | | | | |
| | | | miR-100HG Promoter | | | 1 | | 2 | | | | | |
| | | | miR-100HG Enhancer | | 3 | 1 | 4 | | 1 | | | | |
| | | | miR-100HG Promoter | low H3K4me3 signal | | | | | | | | | 1 |
| | | | miR-100HG Enhancer | | | | | 1 | | 1 | | | 2 |
| | | | miR-100HG Promoter | | | 1 | | | 1 | 1 | | | |
| | | | miR-100HG Enhancer | | | 1 | | | 1 | 1 | | | 1 |
| | | | miR-100HG Promoter | low H3K4me3 signal | | | | 1 | | | | | |
| | | | miR-100HG Enhancer | | | | | 1 | | | | | |
| | | | miR-100HG Promoter | | | | | | | | | | |
| | | | miR-100HG Promoter | | | | | | | | | | |
| | | | miR-100HG Enhancer | | 1 | | | 4 | | | | | |
| | | | miR-100 promoter | | | | | 1 | | | | | |
| | | | miR-100 enhancer | | 1 | | | 3 | | | | | |
| hsa-miR-329-1 | 14q32.3 | independent | miR-329-1 promoter | low H3K4me3 signal | | | | | 2 | | | | |
| | | | miR-329-1 enhancer | low histone mark | | | | | 3 | | | | |
| | | | | signals | | | | | | | | | |
| hsa-miR-454 | 17q22 | SKA2 | SKA2 promoter | | 1 | 1 | | 1 | 1 | 2 | | | 1 |
| | | | SKA2 enhancer | | 1 | 1 | 1 | 4 | 3 | 4 | 1 | 1 | 1 |
| | | | miR-454 promoter | | | | | 1 | | | | | |
| | | | miR-454 enhancer | | | | | 1 | 2 | | 1 | | |

| | | | | SUMMARY | FOS | HDAC2 | IRF1 | JUN | JUNB | KAT2B | KDM5B |
|---|---|---|---|---|---|---|---|---|---|---|---|
| | | | | Total # of binding | 147 | 96 | 53 | 69 | 41 | 10 | 53 |
| | | | | sites in all miRNAs | | | | | | | |
| promoter = within 2 kb | | | | | | | | | | | |
| enhancer = within 10 kb | | | | | | | | | | | |
| microRNA | locus | Home Gene | Promoter/Enhance | Notes | FOS | HDAC2 | IRF1 | JUN | JUNB | KAT2B | KDM5B |
| hsa-miR-32 | 9q31.3 | TMEM245 | TMEM245 promoter | | 1 | 1 | 1 | 1 | 1 | | 1 |
| | | | TMEM245 enhancer | | 5 | 5 | 5 | 3 | 4 | | 1 |
| | | | TMEM245 promoter | low H3K4me3 signal | 1 | 1 | 1 | | | | |
| | | | TMEM245 enhancer | | 6 | 4 | 4 | 3 | 5 | | |
| | | | TMEM245 promoter | low H3K4me3 signal | 1 | | | | | | |
| | | | TMEM245 enhancer | | 2 | 1 | | | 2 | | |
| | | | miR-32 promoter | low H3K4me3 signal | | | | | | | |
| | | | miR-32 enhancer | | 4 | 1 | | 1 | | | |
| hsa-miR-138-1 | 3p21.3 | independent | miR-138-1 promoter | low H3K4me3 signal | 1 | | | | | 1 | |
| | | | miR-138-1 enhancer | | 3 | | | 1 | 1 | 1 | |
| hsa-miR-103-1 | 5q34 | PANK3 | PANK3 promoter | | 1 | | 2 | 1 | | | 1 |
| | | | PANK3 enhancer | | 3 | 1 | 2 | 2 | 2 | | 2 |
| | | | PANK3 promoter | | 1 | | 2 | 1 | | | 2 |
| | | | PANK3 enhancer | | 3 | 1 | 2 | 2 | 2 | | 2 |
| | | | miR-103-1 promoter | low H3K4me3 signal | | | | | | | |
| | | | miR-103-1 enhancer | | 2 | | | | | | |
| hsa-miR-505 | Xq27.1 | ATP11C | ATP11C promoter | | | | 1 | | | | 1 |
| | | | ATP11C enhancer | | 1 | | 1 | | | | 2 |
| | | | miR-505 promoter | low H3K4me3 signal | | | | | | | |
| | | | miR-505 enhancer | | | | 1 | | | | 2 |
| hsa-miR-146b | 10q24.3 | independent | miR-146b promoter | | 1 | 1 | 1 | | | | 1 |
| | | | miR-146b enhancer | | 3 | 3 | 2 | 1 | | | 3 |
| hsa-miR-29b-1 | 7q32.3 | LOC646329 | LOC646329 promoter | | 3 | 1 | 1 | 3 | 1 | | |
| | | | LOC646329 enhancer | | 9 | 5 | 1 | 8 | 1 | | |
| | | | miR-29b-1 promoter | low H3K4me3 signal | | | | | | | |
| | | | miR-29b-1 enhancer | | 3 | 1 | | 1 | | | |
| hsa-miR-486 | 8p11.2 | NKX6-3 and | NKX6-3 promoter | low H3K4me3 signal | | | | | | | |
| | | ANK1 | | | | | | | | | |
| | | | NKX6-3 enhancer | | 2 | 3 | 1 | 2 | 1 | | 1 |
| | | | ANK1 promoter | | | | | | | | |
| | | | ANK1 enhancer | | 2 | | | 1 | | | |
| | | | ANK1 promoter | low H3K4me3 signal | | | | | | | |
| | | | ANK1 enhancer | | 2 | | | 1 | 1 | | |
| | | | ANK1 promoter | | 2 | 2 | 1 | 2 | 1 | | 2 |
| | | | ANK1 enhancer | | 5 | 7 | 2 | 5 | 2 | | 3 |
| | | | ANK1 promoter | | | | | | | | |
| | | | ANK1 enhancer | | | 2 | | | | | |
| | | | miR-486 promoter | low H3K4me3 signal | | | | | | | |
| | | | miR-486 enhancer | | 2 | | | 1 | | | |
| hsa-miR-34a | 1p36.23 | mir-34aHG | mir-34aHG Promoter | | | | | | | | |
| | | | mir-34aHG Enhancer | | 1 | 1 | | | | 1 | |
| | | | miR-34a promoter | low H3K4me3 signal | | | | | | | |
| | | | miR-34a enhancer | | 1 | | | 1 | 1 | | |
| hsa-miR-141 | 12p13.3 | miR-200C HG | miR-200C HG promoter | | 2 | 2 | | 1 | 1 | | 1 |
| | | | miR-141 promoter | | 1 | 1 | | 1 | | | 1 |
| | | | miR-141/miR-200C HG | miRNAs share | 3 | 6 | 1 | 3 | 3 | | 2 |
| | | | enhancer | regulatory elements | | | | | | | |
| hsa-miR-98 | Xp11.2 | HUWE1 | HUWE1 promoter | low H3K4me3 signal | 1 | | | | | | |
| | | | HUWE1 enhancer | | 2 | | | | 2 | | |
| | | | HUWE1 promoter | | | | | | | | |
| | | | HUWE1 enhancer | | 1 | | 1 | 1 | | | |
| | | | miR-98 promoter | low H3K4me3 signal | | | | | | | |
| | | | miR-98 enhancer | | 1 | | | | | | |
| hsa-miR-576 | 4q25 | SEC24B | SEC24B promoter | | 1 | 1 | 1 | 1 | | 1 | 1 |
| | | | SEC24B enhancer | | 6 | 1 | 1 | 1 | 1 | 1 | 1 |
| | | | miR-576 promoter | low H3K4me3 signal | | | | | | | |
| | | | miR-576 enhancer | | | | | | | | |
| hsa-miR-9-1 | 1q22 | C1orf61 | C1orf61 Promoter | low H3K4me3 signal | | | | | | | |
| | | | C1orf61 Enhancer | | 1 | 1 | 1 | 2 | | | 2 |
| | | | C1orf61 Promoter | | 1 | 1 | 1 | 1 | | | 1 |
| | | | C1orf61 Enhancer | | 1 | 2 | 1 | 2 | | | 2 |
| | | | miR-9-1 promoter | | | | 1 | 2 | | | 2 |
| | | | miR-9-1 enhancer | | 1 | 2 | 1 | 2 | | | 2 |
| hsa-miR-142 | 17q22 | independent | miR-142 promoter | | | 2 | 2 | 2 | 1 | | 2 |
| | | | miR-142 enhancer | | 2 | 8 | 7 | 6 | 4 | | 6 |
| hsa-miR-92a-1 | 13q31.3 | miR-17HG/ | miR-17 promoter | | | 1 | | | 1 | | |
| | | miR-19B1 | | | | | | | | | |
| | | | miR-17 HG enhancer | | 1 | 3 | | | 1 | | |
| | | | miR-19B1 promoter | | | 1 | | | | | |
| | | | miR-92a-1 promoter | | | 1 | | | | | |
| | | | miR-19B1/miR-92a-1 | share regulatory | 1 | 2 | | | 1 | | |
| | | | | elements | | | | | | | |
| hsa-miR-502 | Xp11.2 | CLCN5 | CLCN5 promoter | | | 2 | | | | | |
| | | | CLCN5 enhancer | | | 3 | | | | | |
| | | | miR-502 promoter | low H3K4me3 signal | 1 | | | | | | |
| | | | miR-502 promoter | | 1 | | | | | | |
| hsa-miR-140 | 16q22.1 | WWP2 | WWP2 Promoter | | 1 | 1 | | | | | 1 |
| | | | WWP2 Enhancer | | 1 | 4 | 1 | 1 | | | 2 |
| | | | WWP2 Promoter | low H3K4me3 signal | | | | | | | |
| | | | WWP2 Enhancer | | 2 | 4 | | | | | |
| | | | WWP2 Promoter | low H3K4me3 signal | | | | | | | |
| | | | WWP2 Enhancer | | 1 | | | | | | |
| | | | WWP2 Promoter | High H3K4me3 | | | | | | | |
| | | | WWP2 Enhancer | | 1 | | | | | | |
| | | | miR-140 promoter | low H3K4me3 signal | | | | | | | |
| | | | miR-140 enhancer | | | | | | | | |
| hsa-miR-100 | 11q24.1 | miR-100HG | miR-100HG Promoter | | 1 | | | | | | |
| | | | miR-100HG Enhancer | | 8 | | | | 1 | | |
| | | | miR-100HG Promoter | | 3 | | | | | | |
| | | | miR-100HG Enhancer | | 9 | | | | | | |
| | | | miR-100HG Promoter | low H3K4me3 signal | | | | | | | |
| | | | miR-100HG Enhancer | | | | | | | | |
| | | | miR-100HG Promoter | | 1 | | | | | | |
| | | | miR-100HG Enhancer | | 2 | | | | | | |
| | | | miR-100HG Promoter | low H3K4me3 signal | 1 | | | | | | |
| | | | miR-100HG Enhancer | | 3 | | | | | | |
| | | | miR-100HG Promoter | | 1 | | | | | | |
| | | | miR-100HG Promoter | | 1 | | | | | | |
| | | | miR-100HG Enhancer | | 5 | 1 | | | | | |
| | | | miR-100 promoter | | | | | | | | |
| | | | miR-100 enhancer | | 3 | 1 | | | | | |
| hsa-miR-329-1 | 14q32.3 | independent | miR-329-1 promoter | low H3K4me3 signal | | | | | | | |
| | | | miR-329-1 enhancer | low histone mark | | | | | | | |
| | | | | signals | | | | | | | |
| hsa-miR-454 | 17q22 | SKA2 | SKA2 promoter | | 1 | 1 | 1 | 1 | | | 1 |
| | | | SKA2 enhancer | | 3 | 3 | 2 | 1 | | 1 | 2 |
| | | | miR-454 promoter | | | | | | | | |
| | | | miR-454 enhancer | | | | | | | | |

| | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
|---|---|---|---|---|---|---|---|---|---|---|---|---|---|---|---|---|
| | 30 | 127 | 41 | 2 | 10 | 66 | 6 | 2 | 49 | 52 | 42 | 35 | 12 | 4 | 81 | 23 |
| microRNA | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
| hsa-miR-32 | 1 | 2 | | | | 2 | | | 1 | 1 | 1 | 1 | | | 1 | 1 |
| | 1 | 5 | 1 | | | 2 | | | 2 | 1 | 1 | 3 | | | 4 | 1 |
| | | 3 | 1 | | | | | | 1 | | 1 | 2 | | | 4 | |
| | | | | | | | | | | | 1 | | | | 1 | |
| | | | | | | | | | 1 | | | | | | 2 | |
| hsa-miR-138-1 | | 1 | | | | | | | | | | | | | | |
| | | 2 | | | | | | | | | | | | | | |
| hsa-miR-103-1 | 1 | 1 | 1 | | | | | | | 1 | 1 | 1 | | | 1 | 1 |
| | 1 | 2 | 1 | | 1 | 1 | | | 1 | 1 | 3 | 1 | 1 | | 2 | 1 |
| | 1 | 1 | 1 | | 1 | 1 | | | | 1 | 1 | 1 | 1 | | 1 | 1 |
| | 1 | 2 | 1 | | 1 | 1 | | | 1 | 1 | 3 | 1 | 1 | | 2 | 1 |
| hsa-miR-505 | 1 | 1 | | | | 1 | | | | 1 | | | | | 1 | 1 |
| | 1 | 2 | | | | 1 | | | 2 | 1 | | | | | 1 | |
| | 1 | 2 | | | | 1 | | | 2 | 1 | | | | | 1 | 1 |

| | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
|---|---|---|---|---|---|---|---|---|---|---|---|---|---|---|---|---|
| | 11 | 21 | 7 | 39 | 53 | 18 | 3 | 164 | 16 | 30 | 3 | 49 | 45 | 51 | 1 | 7 |
| microRNA | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
| hsa-miR-32 | | 1 | | | 1 | | | 1 | | 1 | 1 | 2 | 1 | 1 | | |
| | | 1 | 1 | | 1 | 1 | | 6 | | 1 | 1 | 3 | 1 | 1 | | |
| | | 1 | | | | | | 6 | | | | | | 1 | | |
| | | | | | | | | 1 | | | | | | 1 | | |
| | | | | | | | | 1 | 1 | | | | | | | |
| hsa-miR-138-1 | | | | | | | | 1 | | | | | | | | |
| | | | | | | | | 1 | | | | | | | | |
| hsa-miR-103-1 | | | | | | | | 1 | | | | | 1 | 1 | | |
| | | | | | | 1 | | 4 | | | | 1 | 1 | 1 | | |
| | | | | | | 1 | | 2 | | | | | 1 | 1 | | |
| | | | | | | 1 | | 4 | | | | 1 | 1 | 1 | | |
| | | | | | | | | 1 | 1 | | | | | | | |
| hsa-miR-505 | | | | | | | | 1 | | | | | | | | |
| | | | | | | 1 | | 3 | | 1 | | | | 2 | | |
| | | | | | | 1 | | 3 | | 1 | | | | 2 | | |

| SUMMARY | ARID3A | ATF3 | BCL3 | CEBPB | CREB1 | EGR1 | ETS1 | ETV6 | EZH2 |
|---|---|---|---|---|---|---|---|---|---|
| Total # of binding | 73 | 93 | 56 | 115 | 61 | 104 | 70 | 28 | 46 |
| sites in all miRNAs | | | | | | | | | |

promoter = within 2 kb
enhancer = within 10 kb

| microRNA | locus | Home Gene | Promoter/Enhance | Notes | ARID3A | ATF3 | BCL3 | CEBPB | CREB1 | EGR1 | ETS1 | ETV6 | EZH2 |
|---|---|---|---|---|---|---|---|---|---|---|---|---|---|
| hsa-miR-32 | 9q31.3 | TMEM245 | TMEM245 promoter | | 1 | 1 | | | 1 | 2 | 1 | | |
| | | | TMEM245 enhancer | | 5 | 4 | | 5 | 2 | 3 | 1 | | |
| | | | TMEM245 promoter | low H3K4me3 signal | 1 | 1 | | 1 | | | | | |
| | | | TMEM245 enhancer | | 5 | 4 | 1 | 4 | 1 | | | 2 | |
| | | | TMEM245 promoter | low H3K4me3 signal | | | | 1 | | | | | |
| | | | TMEM245 enhancer | | 1 | 1 | 1 | 2 | | | | | |
| | | | miR-32 promoter | low H3K4me3 signal | | | | | | | | | |
| | | | miR-32 enhancer | | 1 | 2 | 1 | 1 | | 1 | 1 | | |
| hsa-miR-138-1 | 3p21.3 | independent | miR-138-1 promoter | low H3K4me3 signal | | | | | | | | | |
| | | | miR-138-1 enhancer | | | 1 | 2 | | 1 | | | | |
| hsa-miR-103-1 | 5q34 | PANK3 | PANK3 promoter | | 1 | 1 | | | 1 | 1 | 1 | | |
| | | | PANK3 enhancer | | 1 | 2 | | 3 | 3 | 1 | 1 | | |
| | | | PANK3 promoter | | 1 | 1 | | | 1 | 1 | 1 | | |
| | | | PANK3 enhancer | | 1 | 2 | | 3 | 3 | 1 | 1 | | |
| | | | miR-103-1 promoter | low H3K4me3 signal | | | | | | | | | |
| | | | miR-103-1 enhancer | | | | | 1 | | | | | |
| hsa-miR-505 | Xq27.1 | ATP11C | ATP11C promoter | | | | | | | 1 | 1 | | |
| | | | ATP11C enhancer | | 1 | | | 1 | | 2 | 1 | | |
| | | | miR-505 promoter | low H3K4me3 signal | | | | 1 | | | | | |
| | | | miR-505 enhancer | | 1 | | | 1 | | 2 | 1 | | |
| hsa-miR-146b | 10q24.3 | independent | miR-146b promoter | | | 2 | | 1 | 1 | 2 | | | 1 |
| | | | miR-146b enhancer | | | 4 | 1 | 3 | 2 | 4 | 1 | | 1 |
| hsa-miR-29b-1 | 7q32.3 | LOC646329 | LOC646329 promoter | | 1 | 1 | 1 | 2 | | 1 | 1 | 1 | |
| | | | LOC646329 enhancer | | 6 | 9 | 5 | 10 | 2 | 3 | 6 | 1 | |
| | | | miR-29b-1 promoter | low H3K4me3 signal | | | | | | | | | |
| | | | miR-29b-1 enhancer | | | 4 | 4 | 4 | | 2 | 3 | | |
| hsa-miR-486 | 8p11.2 | NKX6-3 and | NKX6-3 promoter | low H3K4me3 signal | | | | | | | | | |
| | | ANK1 | | | | | | | | | | | |
| | | | NKX6-3 enhancer | | 1 | 2 | | | | 3 | 1 | | |
| | | | ANK1 promoter | | | | | | | | | | |
| | | | ANK1 enhancer | | | 1 | 1 | | 1 | | | | |
| | | | ANK1 promoter | low H3K4me3 signal | | | | | | | | | |
| | | | ANK1 enhancer | | 3 | 2 | 2 | 2 | | | 1 | 1 | 1 |
| | | | ANK1 promoter | | | 1 | | 2 | | 3 | 2 | | 1 |
| | | | ANK1 enhancer | | 1 | 4 | | 2 | | 6 | 3 | | 1 |
| | | | ANK1 promoter | | | | | | | | | | 2 |
| | | | ANK1 enhancer | | | 1 | | 1 | 2 | | | | 4 |
| | | | miR-486 promoter | low H3K4me3 signal | | | | | | | | | |
| | | | miR-486 enhancer | | | 1 | 1 | | | 1 | | | 1 |
| hsa-miR-34a | 1p36.23 | mir-34aHG | mir-34aHG Promoter | | 1 | | 1 | | | | | | 1 |
| | | | mir-34aHG Enhancer | | 2 | | 2 | 1 | | | | | 1 |
| | | | miR-34a promoter | low H3K4me3 signal | | 1 | | | | | | | |
| | | | miR-34a enhancer | | | 3 | | 3 | 1 | 1 | | | |
| hsa-miR-141 | 12p13.3 | miR-200C HG | miR-200C HG promoter | | | 1 | 1 | 1 | 1 | 2 | 2 | 1 | |
| | | | miR-141 promoter | | | | 1 | 1 | | 1 | 2 | | |
| | | | miR-141/miR-200C HG | miRNAs share | 3 | 2 | 5 | 3 | 2 | 6 | 6 | 4 | |
| | | | enhancer | regulatory elements | | | | | | | | | |
| hsa-miR-98 | Xp11.2 | HUWE1 | HUWE1 promoter | low H3K4me3 signal | | | | 1 | | | | 1 | |
| | | | HUWE1 enhancer | | | | | 1 | | | | 1 | |
| | | | HUWE1 promoter | | | | | | | | | | |
| | | | HUWE1 enhancer | | 1 | | 1 | | 1 | 3 | | | |
| | | | miR-98 promoter | low H3K4me3 signal | | | | | | | | | |
| | | | miR-98 enhancer | | | | | | | | | | |
| hsa-miR-576 | 4q25 | SEC24B | SEC24B promoter | | 2 | | | 1 | 1 | 1 | 1 | | |
| | | | SEC24B enhancer | | 3 | 2 | | 3 | 1 | 3 | 1 | | |
| | | | miR-576 promoter | low H3K4me3 signal | | | | | | | | | |
| | | | miR-576 enhancer | | | | | | | | | | |
| hsa-miR-9-1 | 1q22 | C1orf61 | C1orf61 Promoter | low H3K4me3 signal | | | | | 1 | 2 | | | 2 |
| | | | C1orf61 Enhancer | | | 2 | 1 | 1 | 3 | 3 | | 1 | 5 |
| | | | C1orf61 Promoter | | | 2 | | 1 | 1 | 1 | | | 1 |
| | | | C1orf61 Enhancer | | | 2 | 1 | 1 | 2 | 4 | | | 6 |
| | | | miR-9-1 promoter | | | 1 | | | 1 | 1 | | | 1 |
| | | | miR-9-1 enhancer | | | 2 | 1 | 1 | 2 | 4 | | | 5 |
| hsa-miR-142 | 17q22 | independent | miR-142 promoter | | 1 | 2 | 3 | | | 3 | 3 | 3 | |
| | | | miR-142 enhancer | | 4 | 5 | 9 | 6 | 3 | 9 | 11 | 11 | 3 |
| hsa-miR-92a-1 | 13q31.3 | miR-17HG/ | miR-17 promoter | | 1 | | | | | | 1 | | 1 |
| | | miR-19B1 | | | | | | | | | | | |
| | | | miR-17 HG enhancer | | 5 | 1 | 2 | 3 | 1 | 2 | 4 | | 1 |
| | | | miR-19B1 promoter | | 2 | | 1 | | | 1 | 2 | | |
| | | | miR-92a-1 promoter | | | | | | | | 1 | | |
| | | | miR-19B1/miR-92a-1 | share regulatory | 4 | | 2 | 1 | | 2 | 2 | | 1 |
| | | | | elements | | | | | | | | | |
| hsa-miR-502 | Xp11.2 | CLCN5 | CLCN5 promoter | | | | | | | | | | |
| | | | CLCN5 enhancer | | | | | 1 | | 1 | | | |
| | | | miR-502 promoter | low H3K4me3 signal | | | | | | | | | |
| | | | miR-502 promoter | | | | | | | | | | |
| hsa-miR-140 | 16q22.1 | WWP2 | WWP2 Promoter | | 1 | 1 | | | | 1 | 1 | | |
| | | | WWP2 Enhancer | | 2 | 3 | | 2 | 1 | 4 | 2 | | |
| | | | WWP2 Promoter | low H3K4me3 signal | | | | | | | | | |
| | | | WWP2 Enhancer | | 2 | | | 3 | | | | | |
| | | | WWP2 Promoter | low H3K4me3 signal | | | | | | | | | |
| | | | WWP2 Enhancer | | | | | 1 | | | | | |
| | | | WWP2 Promoter | High H3K4me3 | | | | | | | | | |
| | | | WWP2 Enhancer | | | | | 1 | | | | | |
| | | | miR-140 promoter | low H3K4me3 signal | | | | | | | | | |
| | | | miR-140 enhancer | | | | | 1 | | | 1 | | |
| hsa-miR-100 | 11q24.1 | miR-100HG | miR-100HG Promoter | | | 1 | | | 2 | | | | |
| | | | miR-100HG Enhancer | | | 4 | | 6 | 2 | | | | |
| | | | miR-100HG Promoter | | | 1 | | 2 | | | | | |
| | | | miR-100HG Enhancer | | 3 | 1 | 4 | | 1 | | | | |
| | | | miR-100HG Promoter | low H3K4me3 signal | | | | | | | | | 1 |
| | | | miR-100HG Enhancer | | | | | 1 | | 1 | | | 2 |
| | | | miR-100HG Promoter | | | 1 | | | 1 | 1 | | | |
| | | | miR-100HG Enhancer | | | 1 | | | 1 | 1 | | | 1 |
| | | | miR-100HG Promoter | low H3K4me3 signal | | | | 1 | | | | | |
| | | | miR-100HG Enhancer | | | | | 1 | | | | | |
| | | | miR-100HG Promoter | | | | | | | | | | |
| | | | miR-100HG Promoter | | | | | | | | | | |
| | | | miR-100HG Enhancer | | 1 | | | 4 | | | | | |
| | | | miR-100 promoter | | | | | 1 | | | | | |
| | | | miR-100 enhancer | | 1 | | | 3 | | | | | |
| hsa-miR-329-1 | 14q32.3 | independent | miR-329-1 promoter | low H3K4me3 signal | | | | | 2 | | | | |
| | | | miR-329-1 enhancer | low histone mark | | | | | 3 | | | | |
| | | | | signals | | | | | | | | | |
| hsa-miR-454 | 17q22 | SKA2 | SKA2 promoter | | 1 | 1 | | 1 | 1 | 2 | | | 1 |
| | | | SKA2 enhancer | | 1 | 1 | 1 | 4 | 3 | 4 | 1 | 1 | 1 |
| | | | miR-454 promoter | | | | | 1 | | | | | |
| | | | miR-454 enhancer | | | | | 1 | 2 | | 1 | | |

| | | | | SUMMARY | FOS | HDAC2 | IRF1 | JUN | JUNB | KAT2B | KDM5B |
|---|---|---|---|---|---|---|---|---|---|---|---|
| | | | | Total # of binding | 147 | 96 | 53 | 69 | 41 | 10 | 53 |
| | | | | sites in all miRNAs | | | | | | | |
| promoter = within 2 kb | | | | | | | | | | | |
| enhancer = within 10 kb | | | | | | | | | | | |
| microRNA | locus | Home Gene | Promoter/Enhance | Notes | FOS | HDAC2 | IRF1 | JUN | JUNB | KAT2B | KDM5B |
| hsa-miR-32 | 9q31.3 | TMEM245 | TMEM245 promoter | | 1 | 1 | 1 | 1 | 1 | | 1 |
| | | | TMEM245 enhancer | | 5 | 5 | 5 | 3 | 4 | | 1 |
| | | | TMEM245 promoter | low H3K4me3 signal | 1 | 1 | 1 | | | | |
| | | | TMEM245 enhancer | | 6 | 4 | 4 | 3 | 5 | | |
| | | | TMEM245 promoter | low H3K4me3 signal | 1 | | | | | | |
| | | | TMEM245 enhancer | | 2 | 1 | | | 2 | | |
| | | | miR-32 promoter | low H3K4me3 signal | | | | | | | |
| | | | miR-32 enhancer | | 4 | 1 | | 1 | | | |
| hsa-miR-138-1 | 3p21.3 | independent | miR-138-1 promoter | low H3K4me3 signal | 1 | | | | | 1 | |
| | | | miR-138-1 enhancer | | 3 | | | 1 | 1 | 1 | |
| hsa-miR-103-1 | 5q34 | PANK3 | PANK3 promoter | | 1 | | 2 | 1 | | | 1 |
| | | | PANK3 enhancer | | 3 | 1 | 2 | 2 | 2 | | 2 |
| | | | PANK3 promoter | | 1 | | 2 | 1 | | | 2 |
| | | | PANK3 enhancer | | 3 | 1 | 2 | 2 | 2 | | 2 |
| | | | miR-103-1 promoter | low H3K4me3 signal | | | | | | | |
| | | | miR-103-1 enhancer | | 2 | | | | | | |
| hsa-miR-505 | Xq27.1 | ATP11C | ATP11C promoter | | | | 1 | | | | 1 |
| | | | ATP11C enhancer | | 1 | | 1 | | | | 2 |
| | | | miR-505 promoter | low H3K4me3 signal | | | | | | | |
| | | | miR-505 enhancer | | | | 1 | | | | 2 |
| hsa-miR-146b | 10q24.3 | independent | miR-146b promoter | | 1 | 1 | 1 | | | | 1 |
| | | | miR-146b enhancer | | 3 | 3 | 2 | 1 | | | 3 |
| hsa-miR-29b-1 | 7q32.3 | LOC646329 | LOC646329 promoter | | 3 | 1 | 1 | 3 | 1 | | |
| | | | LOC646329 enhancer | | 9 | 5 | 1 | 8 | 1 | | |
| | | | miR-29b-1 promoter | low H3K4me3 signal | | | | | | | |
| | | | miR-29b-1 enhancer | | 3 | 1 | | 1 | | | |
| hsa-miR-486 | 8p11.2 | NKX6-3 and | NKX6-3 promoter | low H3K4me3 signal | | | | | | | |
| | | ANK1 | | | | | | | | | |
| | | | NKX6-3 enhancer | | 2 | 3 | 1 | 2 | 1 | | 1 |
| | | | ANK1 promoter | | | | | | | | |
| | | | ANK1 enhancer | | 2 | | | 1 | | | |
| | | | ANK1 promoter | low H3K4me3 signal | | | | | | | |
| | | | ANK1 enhancer | | 2 | | | 1 | 1 | | |
| | | | ANK1 promoter | | 2 | 2 | 1 | 2 | 1 | | 2 |
| | | | ANK1 enhancer | | 5 | 7 | 2 | 5 | 2 | | 3 |
| | | | ANK1 promoter | | | | | | | | |
| | | | ANK1 enhancer | | | 2 | | | | | |
| | | | miR-486 promoter | low H3K4me3 signal | | | | | | | |
| | | | miR-486 enhancer | | 2 | | | 1 | | | |
| hsa-miR-34a | 1p36.23 | mir-34aHG | mir-34aHG Promoter | | | | | | | | |
| | | | mir-34aHG Enhancer | | 1 | 1 | | | | 1 | |
| | | | miR-34a promoter | low H3K4me3 signal | | | | | | | |
| | | | miR-34a enhancer | | 1 | | | 1 | 1 | | |
| hsa-miR-141 | 12p13.3 | miR-200C HG | miR-200C HG promoter | | 2 | 2 | | 1 | 1 | | 1 |
| | | | miR-141 promoter | | 1 | 1 | | 1 | | | 1 |
| | | | miR-141/miR-200C HG | miRNAs share | 3 | 6 | 1 | 3 | 3 | | 2 |
| | | | enhancer | regulatory elements | | | | | | | |
| hsa-miR-98 | Xp11.2 | HUWE1 | HUWE1 promoter | low H3K4me3 signal | 1 | | | | | | |
| | | | HUWE1 enhancer | | 2 | | | | 2 | | |
| | | | HUWE1 promoter | | | | | | | | |
| | | | HUWE1 enhancer | | 1 | | 1 | 1 | | | |
| | | | miR-98 promoter | low H3K4me3 signal | | | | | | | |
| | | | miR-98 enhancer | | 1 | | | | | | |
| hsa-miR-576 | 4q25 | SEC24B | SEC24B promoter | | 1 | 1 | 1 | 1 | | 1 | 1 |
| | | | SEC24B enhancer | | 6 | 1 | 1 | 1 | 1 | 1 | 1 |
| | | | miR-576 promoter | low H3K4me3 signal | | | | | | | |
| | | | miR-576 enhancer | | | | | | | | |
| hsa-miR-9-1 | 1q22 | C1orf61 | C1orf61 Promoter | low H3K4me3 signal | | | | | | | |
| | | | C1orf61 Enhancer | | 1 | 1 | 1 | 2 | | | 2 |
| | | | C1orf61 Promoter | | 1 | 1 | 1 | 1 | | | 1 |
| | | | C1orf61 Enhancer | | 1 | 2 | 1 | 2 | | | 2 |
| | | | miR-9-1 promoter | | | | 1 | 2 | | | 2 |
| | | | miR-9-1 enhancer | | 1 | 2 | 1 | 2 | | | 2 |
| hsa-miR-142 | 17q22 | independent | miR-142 promoter | | | 2 | 2 | 2 | 1 | | 2 |
| | | | miR-142 enhancer | | 2 | 8 | 7 | 6 | 4 | | 6 |
| hsa-miR-92a-1 | 13q31.3 | miR-17HG/ | miR-17 promoter | | | 1 | | | 1 | | |
| | | miR-19B1 | | | | | | | | | |
| | | | miR-17 HG enhancer | | 1 | 3 | | | 1 | | |
| | | | miR-19B1 promoter | | | 1 | | | | | |
| | | | miR-92a-1 promoter | | | 1 | | | | | |
| | | | miR-19B1/miR-92a-1 | share regulatory | 1 | 2 | | | 1 | | |
| | | | | elements | | | | | | | |
| hsa-miR-502 | Xp11.2 | CLCN5 | CLCN5 promoter | | | 2 | | | | | |
| | | | CLCN5 enhancer | | | 3 | | | | | |
| | | | miR-502 promoter | low H3K4me3 signal | 1 | | | | | | |
| | | | miR-502 promoter | | 1 | | | | | | |
| hsa-miR-140 | 16q22.1 | WWP2 | WWP2 Promoter | | 1 | 1 | | | | | 1 |
| | | | WWP2 Enhancer | | 1 | 4 | 1 | 1 | | | 2 |
| | | | WWP2 Promoter | low H3K4me3 signal | | | | | | | |
| | | | WWP2 Enhancer | | 2 | 4 | | | | | |
| | | | WWP2 Promoter | low H3K4me3 signal | | | | | | | |
| | | | WWP2 Enhancer | | 1 | | | | | | |
| | | | WWP2 Promoter | High H3K4me3 | | | | | | | |
| | | | WWP2 Enhancer | | 1 | | | | | | |
| | | | miR-140 promoter | low H3K4me3 signal | | | | | | | |
| | | | miR-140 enhancer | | | | | | | | |
| hsa-miR-100 | 11q24.1 | miR-100HG | miR-100HG Promoter | | 1 | | | | | | |
| | | | miR-100HG Enhancer | | 8 | | | | 1 | | |
| | | | miR-100HG Promoter | | 3 | | | | | | |
| | | | miR-100HG Enhancer | | 9 | | | | | | |
| | | | miR-100HG Promoter | low H3K4me3 signal | | | | | | | |
| | | | miR-100HG Enhancer | | | | | | | | |
| | | | miR-100HG Promoter | | 1 | | | | | | |
| | | | miR-100HG Enhancer | | 2 | | | | | | |
| | | | miR-100HG Promoter | low H3K4me3 signal | 1 | | | | | | |
| | | | miR-100HG Enhancer | | 3 | | | | | | |
| | | | miR-100HG Promoter | | 1 | | | | | | |
| | | | miR-100HG Promoter | | 1 | | | | | | |
| | | | miR-100HG Enhancer | | 5 | 1 | | | | | |
| | | | miR-100 promoter | | | | | | | | |
| | | | miR-100 enhancer | | 3 | 1 | | | | | |
| hsa-miR-329-1 | 14q32.3 | independent | miR-329-1 promoter | low H3K4me3 signal | | | | | | | |
| | | | miR-329-1 enhancer | low histone mark | | | | | | | |
| | | | | signals | | | | | | | |
| hsa-miR-454 | 17q22 | SKA2 | SKA2 promoter | | 1 | 1 | 1 | 1 | | | 1 |
| | | | SKA2 enhancer | | 3 | 3 | 2 | 1 | | 1 | 2 |
| | | | miR-454 promoter | | | | | | | | |
| | | | miR-454 enhancer | | | | | | | | |

| | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
|---|---|---|---|---|---|---|---|---|---|---|---|---|---|---|---|---|
| | 30 | 127 | 41 | 2 | 10 | 66 | 6 | 2 | 49 | 52 | 42 | 35 | 12 | 4 | 81 | 23 |
| microRNA | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
| hsa-miR-32 | 1 | 2 | | | | 2 | | | 1 | 1 | 1 | 1 | | | 1 | 1 |
| | 1 | 5 | 1 | | | 2 | | | 2 | 1 | 1 | 3 | | | 4 | 1 |
| | | 3 | 1 | | | | | | 1 | | 1 | 2 | | | 4 | |
| | | | | | | | | | | | 1 | | | | 1 | |
| | | | | | | | | | 1 | | | | | | 2 | |
| hsa-miR-138-1 | | 1 | | | | | | | | | | | | | | |
| | | 2 | | | | | | | | | | | | | | |
| hsa-miR-103-1 | 1 | 1 | 1 | | | | | | | 1 | 1 | 1 | | | 1 | 1 |
| | 1 | 2 | 1 | | 1 | 1 | | | 1 | 1 | 3 | 1 | 1 | | 2 | 1 |
| | 1 | 1 | 1 | | 1 | 1 | | | | 1 | 1 | 1 | 1 | | 1 | 1 |
| | 1 | 2 | 1 | | 1 | 1 | | | 1 | 1 | 3 | 1 | 1 | | 2 | 1 |
| hsa-miR-505 | 1 | 1 | | | | 1 | | | | 1 | | | | | 1 | 1 |
| | 1 | 2 | | | | 1 | | | 2 | 1 | | | | | 1 | |
| | 1 | 2 | | | | 1 | | | 2 | 1 | | | | | 1 | 1 |

| | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
|---|---|---|---|---|---|---|---|---|---|---|---|---|---|---|---|---|
| | 11 | 21 | 7 | 39 | 53 | 18 | 3 | 164 | 16 | 30 | 3 | 49 | 45 | 51 | 1 | 7 |
| microRNA | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
| hsa-miR-32 | | 1 | | | 1 | | | 1 | | 1 | 1 | 2 | 1 | 1 | | |
| | | 1 | 1 | | 1 | 1 | | 6 | | 1 | 1 | 3 | 1 | 1 | | |
| | | 1 | | | | | | 6 | | | | | | 1 | | |
| | | | | | | | | 1 | | | | | | 1 | | |
| | | | | | | | | 1 | 1 | | | | | | | |
| hsa-miR-138-1 | | | | | | | | 1 | | | | | | | | |
| | | | | | | | | 1 | | | | | | | | |
| hsa-miR-103-1 | | | | | | | | 1 | | | | | 1 | 1 | | |
| | | | | | | 1 | | 4 | | | | 1 | 1 | 1 | | |
| | | | | | | 1 | | 2 | | | | | 1 | 1 | | |
| | | | | | | 1 | | 4 | | | | 1 | 1 | 1 | | |
| | | | | | | | | 1 | 1 | | | | | | | |
| hsa-miR-505 | | | | | | | | 1 | | | | | | | | |
| | | | | | | 1 | | 3 | | 1 | | | | 2 | | |
| | | | | | | 1 | | 3 | | 1 | | | | 2 | | |

| microRNA | locus | Home Gene | Promoter/Enhance | Notes | ARID3A | ATF3 | BCL3 | CEBPB | CREB1 | EGR1 | ETS1 | ETV6 | EZH2 |
|---|---|---|---|---|---|---|---|---|---|---|---|---|---|
| hsa-miR-146b | 10q24.3 | independent | miR-146b promoter | | | 2 | | 1 | 1 | 2 | | | 1 |
| | | | miR-146b enhancer | | | 4 | 1 | 3 | 2 | 4 | 1 | | 1 |
| hsa-miR-29b-1 | 7q32.3 | LOC646329 | LOC646329 promoter | | 1 | 1 | 1 | 2 | | 1 | 1 | 1 | |
| | | | LOC646329 enhancer | | 6 | 9 | 5 | 10 | 2 | 3 | 6 | 1 | |
| | | | miR-29b-1 promoter | low H3K4me3 signal | | | | | | | | | |
| | | | miR-29b-1 enhancer | | | 4 | 4 | 4 | | 2 | 3 | | |
| hsa-miR-486 | 8p11.2 | NKX6-3 and | NKX6-3 promoter | low H3K4me3 signal | | | | | | | | | |
| | | ANK1 | | | | | | | | | | | |
| | | | NKX6-3 enhancer | | 1 | 2 | | | | 3 | 1 | | |
| | | | ANK1 promoter | | | | | | | | | | |
| | | | ANK1 enhancer | | | 1 | 1 | | 1 | | | | |
| | | | ANK1 promoter | low H3K4me3 signal | | | | | | | | | |
| | | | ANK1 enhancer | | 3 | 2 | 2 | 2 | | | 1 | 1 | 1 |
| | | | ANK1 promoter | | | 1 | | 2 | | 3 | 2 | | 1 |
| | | | ANK1 enhancer | | 1 | 4 | | 2 | | 6 | 3 | | 1 |
| | | | ANK1 promoter | | | | | | | | | | 2 |
| | | | ANK1 enhancer | | | 1 | | 1 | 2 | | | | 4 |
| | | | miR-486 promoter | low H3K4me3 signal | | | | | | | | | |
| | | | miR-486 enhancer | | | 1 | 1 | | | 1 | | | 1 |
| hsa-miR-34a | 1p36.23 | mir-34aHG | mir-34aHG Promoter | | 1 | | 1 | | | | | | 1 |
| | | | mir-34aHG Enhancer | | 2 | | 2 | 1 | | | | | 1 |
| | | | miR-34a promoter | low H3K4me3 signal | | 1 | | | | | | | |
| | | | miR-34a enhancer | | | 3 | | 3 | 1 | 1 | | | |
| hsa-miR-141 | 12p13.3 | miR-200C HG | miR-200C HG promoter | | | 1 | 1 | 1 | 1 | 2 | 2 | 1 | |
| | | | miR-141 promoter | | | | 1 | 1 | | 1 | 2 | | |
| | | | miR-141/miR-200C HG | miRNAs share | 3 | 2 | 5 | 3 | 2 | 6 | 6 | 4 | |
| | | | enhancer | regulatory elements | | | | | | | | | |
| hsa-miR-98 | Xp11.2 | HUWE1 | HUWE1 promoter | low H3K4me3 signal | | | | 1 | | | | 1 | |
| | | | HUWE1 enhancer | | | | | 1 | | | | 1 | |
| | | | HUWE1 promoter | | | | | | | | | | |
| | | | HUWE1 enhancer | | 1 | | 1 | | 1 | 3 | | | |
| | | | miR-98 promoter | low H3K4me3 signal | | | | | | | | | |
| | | | miR-98 enhancer | | | | | | | | | | |
| hsa-miR-576 | 4q25 | SEC24B | SEC24B promoter | | 2 | | | 1 | 1 | 1 | 1 | | |
| | | | SEC24B enhancer | | 3 | 2 | | 3 | 1 | 3 | 1 | | |
| | | | miR-576 promoter | low H3K4me3 signal | | | | | | | | | |
| | | | miR-576 enhancer | | | | | | | | | | |
| hsa-miR-9-1 | 1q22 | C1orf61 | C1orf61 Promoter | low H3K4me3 signal | | | | | 1 | 2 | | | 2 |
| | | | C1orf61 Enhancer | | | 2 | 1 | 1 | 3 | 3 | | 1 | 5 |
| | | | C1orf61 Promoter | | | 2 | | 1 | 1 | 1 | | | 1 |
| | | | C1orf61 Enhancer | | | 2 | 1 | 1 | 2 | 4 | | | 6 |
| | | | miR-9-1 promoter | | | 1 | | | 1 | 1 | | | 1 |
| | | | miR-9-1 enhancer | | | 2 | 1 | 1 | 2 | 4 | | | 5 |
| hsa-miR-142 | 17q22 | independent | miR-142 promoter | | 1 | 2 | 3 | | | 3 | 3 | 3 | |
| | | | miR-142 enhancer | | 4 | 5 | 9 | 6 | 3 | 9 | 11 | 11 | 3 |
| hsa-miR-92a-1 | 13q31.3 | miR-17HG/ | miR-17 promoter | | 1 | | | | | | 1 | | 1 |
| | | miR-19B1 | | | | | | | | | | | |
| | | | miR-17 HG enhancer | | 5 | 1 | 2 | 3 | 1 | 2 | 4 | | 1 |
| | | | miR-19B1 promoter | | 2 | | 1 | | | 1 | 2 | | |
| | | | miR-92a-1 promoter | | | | | | | | 1 | | |
| | | | miR-19B1/miR-92a-1 | share regulatory | 4 | | 2 | 1 | | 2 | 2 | | 1 |
| | | | | elements | | | | | | | | | |
| hsa-miR-502 | Xp11.2 | CLCN5 | CLCN5 promoter | | | | | | | | | | |
| | | | CLCN5 enhancer | | | | | 1 | | 1 | | | |
| | | | miR-502 promoter | low H3K4me3 signal | | | | | | | | | |
| | | | miR-502 promoter | | | | | | | | | | |
| hsa-miR-140 | 16q22.1 | WWP2 | WWP2 Promoter | | 1 | 1 | | | | 1 | 1 | | |
| | | | WWP2 Enhancer | | 2 | 3 | | 2 | 1 | 4 | 2 | | |
| | | | WWP2 Promoter | low H3K4me3 signal | | | | | | | | | |
| | | | WWP2 Enhancer | | 2 | | | 3 | | | | | |
| | | | WWP2 Promoter | low H3K4me3 signal | | | | | | | | | |
| | | | WWP2 Enhancer | | | | | 1 | | | | | |
| | | | WWP2 Promoter | High H3K4me3 | | | | | | | | | |
| | | | WWP2 Enhancer | | | | | 1 | | | | | |
| | | | miR-140 promoter | low H3K4me3 signal | | | | | | | | | |
| | | | miR-140 enhancer | | | | | 1 | | | 1 | | |
| hsa-miR-100 | 11q24.1 | miR-100HG | miR-100HG Promoter | | | 1 | | | 2 | | | | |
| | | | miR-100HG Enhancer | | | 4 | | 6 | 2 | | | | |
| | | | miR-100HG Promoter | | | 1 | | 2 | | | | | |
| | | | miR-100HG Enhancer | | 3 | 1 | 4 | | 1 | | | | |
| | | | miR-100HG Promoter | low H3K4me3 signal | | | | | | | | | 1 |
| | | | miR-100HG Enhancer | | | | | 1 | | 1 | | | 2 |
| | | | miR-100HG Promoter | | | 1 | | | 1 | 1 | | | |
| | | | miR-100HG Enhancer | | | 1 | | | 1 | 1 | | | 1 |
| | | | miR-100HG Promoter | low H3K4me3 signal | | | | 1 | | | | | |
| | | | miR-100HG Enhancer | | | | | 1 | | | | | |
| | | | miR-100HG Promoter | | | | | | | | | | |
| | | | miR-100HG Promoter | | | | | | | | | | |
| | | | miR-100HG Enhancer | | 1 | | | 4 | | | | | |
| | | | miR-100 promoter | | | | | 1 | | | | | |
| | | | miR-100 enhancer | | 1 | | | 3 | | | | | |
| hsa-miR-329-1 | 14q32.3 | independent | miR-329-1 promoter | low H3K4me3 signal | | | | | 2 | | | | |
| | | | miR-329-1 enhancer | low histone mark | | | | | 3 | | | | |
| | | | | signals | | | | | | | | | |
| hsa-miR-454 | 17q22 | SKA2 | SKA2 promoter | | 1 | 1 | | 1 | 1 | 2 | | | 1 |
| | | | SKA2 enhancer | | 1 | 1 | 1 | 4 | 3 | 4 | 1 | 1 | 1 |
| | | | miR-454 promoter | | | | | 1 | | | | | |
| | | | miR-454 enhancer | | | | | 1 | 2 | | 1 | | |

| microRNA | locus | Home Gene | Promoter/Enhance | Notes | FOS | HDAC2 | IRF1 | JUN | JUNB | KAT2B | KDM5B |
|---|---|---|---|---|---|---|---|---|---|---|---|
| hsa-miR-146b | 10q24.3 | independent | miR-146b promoter | | 1 | 1 | 1 | | | | 1 |
| | | | miR-146b enhancer | | 3 | 3 | 2 | 1 | | | 3 |
| hsa-miR-29b-1 | 7q32.3 | LOC646329 | LOC646329 promoter | | 3 | 1 | 1 | 3 | 1 | | |
| | | | LOC646329 enhancer | | 9 | 5 | 1 | 8 | 1 | | |
| | | | miR-29b-1 promoter | low H3K4me3 signal | | | | | | | |
| | | | miR-29b-1 enhancer | | 3 | 1 | | 1 | | | |
| hsa-miR-486 | 8p11.2 | NKX6-3 and | NKX6-3 promoter | low H3K4me3 signal | | | | | | | |
| | | ANK1 | | | | | | | | | |
| | | | NKX6-3 enhancer | | 2 | 3 | 1 | 2 | 1 | | 1 |
| | | | ANK1 promoter | | | | | | | | |
| | | | ANK1 enhancer | | 2 | | | 1 | | | |
| | | | ANK1 promoter | low H3K4me3 signal | | | | | | | |
| | | | ANK1 enhancer | | 2 | | | 1 | 1 | | |
| | | | ANK1 promoter | | 2 | 2 | 1 | 2 | 1 | | 2 |
| | | | ANK1 enhancer | | 5 | 7 | 2 | 5 | 2 | | 3 |
| | | | ANK1 promoter | | | | | | | | |
| | | | ANK1 enhancer | | | 2 | | | | | |
| | | | miR-486 promoter | low H3K4me3 signal | | | | | | | |
| | | | miR-486 enhancer | | 2 | | | 1 | | | |
| hsa-miR-34a | 1p36.23 | mir-34aHG | mir-34aHG Promoter | | | | | | | | |
| | | | mir-34aHG Enhancer | | 1 | 1 | | | | 1 | |
| | | | miR-34a promoter | low H3K4me3 signal | | | | | | | |
| | | | miR-34a enhancer | | 1 | | | 1 | 1 | | |
| hsa-miR-141 | 12p13.3 | miR-200C HG | miR-200C HG promoter | | 2 | 2 | | 1 | 1 | | 1 |
| | | | miR-141 promoter | | 1 | 1 | | 1 | | | 1 |
| | | | miR-141/miR-200C HG | miRNAs share | 3 | 6 | 1 | 3 | 3 | | 2 |
| | | | enhancer | regulatory elements | | | | | | | |
| hsa-miR-98 | Xp11.2 | HUWE1 | HUWE1 promoter | low H3K4me3 signal | 1 | | | | | | |
| | | | HUWE1 enhancer | | 2 | | | | 2 | | |
| | | | HUWE1 promoter | | | | | | | | |
| | | | HUWE1 enhancer | | 1 | | 1 | 1 | | | |
| | | | miR-98 promoter | low H3K4me3 signal | | | | | | | |
| | | | miR-98 enhancer | | 1 | | | | | | |
| hsa-miR-576 | 4q25 | SEC24B | SEC24B promoter | | 1 | 1 | 1 | 1 | | 1 | 1 |
| | | | SEC24B enhancer | | 6 | 1 | 1 | 1 | 1 | 1 | 1 |
| | | | miR-576 promoter | low H3K4me3 signal | | | | | | | |
| | | | miR-576 enhancer | | | | | | | | |
| hsa-miR-9-1 | 1q22 | C1orf61 | C1orf61 Promoter | low H3K4me3 signal | | | | | | | |
| | | | C1orf61 Enhancer | | 1 | 1 | 1 | 2 | | | 2 |
| | | | C1orf61 Promoter | | 1 | 1 | 1 | 1 | | | 1 |
| | | | C1orf61 Enhancer | | 1 | 2 | 1 | 2 | | | 2 |
| | | | miR-9-1 promoter | | | | 1 | 2 | | | 2 |
| | | | miR-9-1 enhancer | | 1 | 2 | 1 | 2 | | | 2 |
| hsa-miR-142 | 17q22 | independent | miR-142 promoter | | | 2 | 2 | 2 | 1 | | 2 |
| | | | miR-142 enhancer | | 2 | 8 | 7 | 6 | 4 | | 6 |
| hsa-miR-92a-1 | 13q31.3 | miR-17HG/ | miR-17 promoter | | | 1 | | | 1 | | |
| | | miR-19B1 | | | | | | | | | |
| | | | miR-17 HG enhancer | | 1 | 3 | | | 1 | | |
| | | | miR-19B1 promoter | | | 1 | | | | | |
| | | | miR-92a-1 promoter | | | 1 | | | | | |
| | | | miR-19B1/miR-92a-1 | share regulatory | 1 | 2 | | | 1 | | |
| | | | | elements | | | | | | | |
| hsa-miR-502 | Xp11.2 | CLCN5 | CLCN5 promoter | | | 2 | | | | | |
| | | | CLCN5 enhancer | | | 3 | | | | | |
| | | | miR-502 promoter | low H3K4me3 signal | 1 | | | | | | |
| | | | miR-502 promoter | | 1 | | | | | | |
| hsa-miR-140 | 16q22.1 | WWP2 | WWP2 Promoter | | 1 | 1 | | | | | 1 |
| | | | WWP2 Enhancer | | 1 | 4 | 1 | 1 | | | 2 |
| | | | WWP2 Promoter | low H3K4me3 signal | | | | | | | |
| | | | WWP2 Enhancer | | 2 | 4 | | | | | |
| | | | WWP2 Promoter | low H3K4me3 signal | | | | | | | |
| | | | WWP2 Enhancer | | 1 | | | | | | |
| | | | WWP2 Promoter | High H3K4me3 | | | | | | | |
| | | | WWP2 Enhancer | | 1 | | | | | | |
| | | | miR-140 promoter | low H3K4me3 signal | | | | | | | |
| | | | miR-140 enhancer | | | | | | | | |
| hsa-miR-100 | 11q24.1 | miR-100HG | miR-100HG Promoter | | 1 | | | | | | |
| | | | miR-100HG Enhancer | | 8 | | | | 1 | | |
| | | | miR-100HG Promoter | | 3 | | | | | | |
| | | | miR-100HG Enhancer | | 9 | | | | | | |
| | | | miR-100HG Promoter | low H3K4me3 signal | | | | | | | |
| | | | miR-100HG Enhancer | | | | | | | | |
| | | | miR-100HG Promoter | | 1 | | | | | | |
| | | | miR-100HG Enhancer | | 2 | | | | | | |
| | | | miR-100HG Promoter | low H3K4me3 signal | 1 | | | | | | |
| | | | miR-100HG Enhancer | | 3 | | | | | | |
| | | | miR-100HG Promoter | | 1 | | | | | | |
| | | | miR-100HG Promoter | | 1 | | | | | | |
| | | | miR-100HG Enhancer | | 5 | 1 | | | | | |
| | | | miR-100 promoter | | | | | | | | |
| | | | miR-100 enhancer | | 3 | 1 | | | | | |
| hsa-miR-329-1 | 14q32.3 | independent | miR-329-1 promoter | low H3K4me3 signal | | | | | | | |
| | | | miR-329-1 enhancer | low histone mark | | | | | | | |
| | | | | signals | | | | | | | |
| hsa-miR-454 | 17q22 | SKA2 | SKA2 promoter | | 1 | 1 | 1 | 1 | | | 1 |
| | | | SKA2 enhancer | | 3 | 3 | 2 | 1 | | 1 | 2 |
| | | | miR-454 promoter | | | | | | | | |
| | | | miR-454 enhancer | | | | | | | | |

| | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
|---|---|---|---|---|---|---|---|---|---|---|---|---|---|---|---|---|
| | 30 | 127 | 41 | 2 | 10 | 66 | 6 | 2 | 49 | 52 | 42 | 35 | 12 | 4 | 81 | 23 |
| microRNA | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
| hsa-miR-32 | 1 | 2 | | | | 2 | | | 1 | 1 | 1 | 1 | | | 1 | 1 |
| | 1 | 5 | 1 | | | 2 | | | 2 | 1 | 1 | 3 | | | 4 | 1 |
| | | 3 | 1 | | | | | | 1 | | 1 | 2 | | | 4 | |
| | | | | | | | | | | | 1 | | | | 1 | |
| | | | | | | | | | 1 | | | | | | 2 | |
| hsa-miR-138-1 | | 1 | | | | | | | | | | | | | | |
| | | 2 | | | | | | | | | | | | | | |
| hsa-miR-103-1 | 1 | 1 | 1 | | | | | | | 1 | 1 | 1 | | | 1 | 1 |
| | 1 | 2 | 1 | | 1 | 1 | | | 1 | 1 | 3 | 1 | 1 | | 2 | 1 |
| | 1 | 1 | 1 | | 1 | 1 | | | | 1 | 1 | 1 | 1 | | 1 | 1 |
| | 1 | 2 | 1 | | 1 | 1 | | | 1 | 1 | 3 | 1 | 1 | | 2 | 1 |
| hsa-miR-505 | 1 | 1 | | | | 1 | | | | 1 | | | | | 1 | 1 |
| | 1 | 2 | | | | 1 | | | 2 | 1 | | | | | 1 | |
| | 1 | 2 | | | | 1 | | | 2 | 1 | | | | | 1 | 1 |
| hsa-miR-146b | 1 | | | | | 1 | | | 1 | 1 | | | | | 2 | |
| | 2 | 2 | | | 1 | 2 | | | 1 | 2 | 1 | | 1 | | 3 | 1 |
| hsa-miR-29b-1 | | 1 | 1 | | | 1 | | | | 1 | 1 | 1 | | | 1 | |
| | 1 | 5 | 5 | | | 3 | | | 3 | 2 | 1 | 1 | | | 9 | 3 |
| | | 3 | 2 | | | | | | | | | | | | 2 | 1 |
| hsa-miR-486 | | | | | | | | | | | | | | | | |
| | | 1 | 1 | | | 2 | | | 4 | | 1 | | | | | |
| | | 1 | | | | | | | 1 | | 3 | | | | | |
| | 2 | 3 | 1 | | 1 | 1 | | | | | | 1 | 1 | 1 | | |
| | 2 | 5 | 2 | | 1 | 3 | | | 4 | | 1 | 1 | 1 | 1 | | |
| | | | | | | 1 | | | | | | | | | | |
| | | 1 | | | | 2 | | | 2 | | | | | | 1 | |
| | | | | | | | | | 1 | | | | | | | |
| hsa-miR-34a | | 1 | | | | | | | | 1 | | | | | | |
| | | 1 | | | | | | | | 2 | 1 | | 1 | | 2 | |
| | | 1 | | | | | | | | | | 1 | | | | |
| hsa-miR-141 | | 3 | 2 | | | 1 | | | | 1 | 1 | | | | 1 | |
| | 1 | 5 | 3 | | | 3 | | | | 2 | 4 | 1 | 1 | | 7 | 1 |
| hsa-miR-98 | | | | | | | | | | | | | | | | |
| | | | | | | | | | | | | 1 | | | | |
| | | | | | | 1 | | | 1 | 1 | | | | | | |
| | 1 | 2 | 2 | | | 2 | | | 3 | 1 | 1 | | 1 | | 1 | |
| hsa-miR-576 | | 1 | | | | 1 | | | 1 | 1 | | | | | 1 | |
| | | 3 | | | | 1 | | | 1 | 1 | 1 | | | | | 2 |
| hsa-miR-9-1 | | | | | | | | | | | | | | | | |
| | | 1 | | | | 3 | | | 1 | 1 | 1 | 1 | | | | |
| | | 1 | | | | 1 | | | 1 | | | 1 | | | | |
| | | 1 | | | | 2 | | | 1 | | 1 | 1 | | | | |
| | | 1 | | | | 2 | | | 1 | | 1 | 1 | | | | |
| | | 1 | | | | 2 | | | 1 | | 1 | 1 | | | | |
| hsa-miR-142 | 1 | 2 | | | | 2 | 2 | 1 | | 1 | | 1 | | | | |
| | 7 | 12 | | | 2 | 4 | 4 | 1 | 1 | 8 | 6 | 5 | 3 | 2 | 3 | 4 |
| hsa-miR-92a-1 | | 1 | | | | 1 | | | | 1 | | 1 | | | | |
| | | 3 | | | | 2 | | | | 2 | | 1 | | | 3 | |
| | | 2 | | | | 1 | | | | 1 | | | | | 1 | |
| | | 1 | | | | | | | | | | | | | | |
| | | 3 | | | | 2 | | | | 2 | | 1 | | | 1 | |
| hsa-miR-502 | | 2 | | | | 1 | | | | | | | | | | |
| | | 2 | | | | 1 | | | | | | | | | | |
| hsa-miR-140 | | 1 | 1 | | | 1 | | | 1 | 1 | | | | | 1 | 1 |
| | 1 | 2 | 3 | | | 2 | | | 3 | 2 | 1 | 1 | | | 2 | 2 |
| | | | | | | | | | | | | | | | 3 | |
| | | 1 | 1 | | | | | | | 1 | | | | | 1 | |
| | | 1 | 1 | | | | | | | 2 | | | | | 1 | |
| | | | | | | | | | | 2 | | | | | 2 | |
| hsa-miR-100 | | | | | | | | | | | | | | | 1 | |
| | | 3 | 2 | | | | | | | | | | | | 4 | |
| | | | 1 | | | | | | | | | | | | | |
| | | 1 | 3 | | | | | | | | | 1 | | | 1 | |
| | | | 1 | | | | | | | | | | | | | |
| | | 1 | | | | | | | | | | | | | 1 | |
| | | 2 | | | | | | | | | | | | | 1 | |
| | | 1 | | | | | | | | | | | | | | |
| | | 2 | | | | | | | | | | | | | 1 | |
| | | 1 | | | | | | | | | | | | | | |
| | | 1 | | | | | | | | | | | | | | |
| | | 3 | | | | | | | | | | | | | | |
| | | 2 | | | | | | | | | | | | | | |
| hsa-miR-329-1 | | | | | | | | | | | | | | | | |
| | | | | | | | | | 1 | | | | | | | |
| hsa-miR-454 | 1 | 1 | | 1 | 1 | 1 | | | 1 | 1 | | 1 | | | 1 | |
| | 1 | 4 | 1 | 1 | 1 | 2 | | | 2 | 1 | 2 | 1 | | | 1 | |
| | | 1 | 1 | | | | | | 1 | | | | | | 1 | |

| | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
|---|---|---|---|---|---|---|---|---|---|---|---|---|---|---|---|---|
| | 11 | 21 | 7 | 39 | 53 | 18 | 3 | 164 | 16 | 30 | 3 | 49 | 45 | 51 | 1 | 7 |
| microRNA | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
| hsa-miR-32 | | 1 | | | 1 | | | 1 | | 1 | 1 | 2 | 1 | 1 | | |
| | | 1 | 1 | | 1 | 1 | | 6 | | 1 | 1 | 3 | 1 | 1 | | |
| | | 1 | | | | | | 6 | | | | | | 1 | | |
| | | | | | | | | 1 | | | | | | 1 | | |
| | | | | | | | | 1 | 1 | | | | | | | |
| hsa-miR-138-1 | | | | | | | | 1 | | | | | | | | |
| | | | | | | | | 1 | | | | | | | | |
| hsa-miR-103-1 | | | | | | | | 1 | | | | | 1 | 1 | | |
| | | | | | | 1 | | 4 | | | | 1 | 1 | 1 | | |
| | | | | | | 1 | | 2 | | | | | 1 | 1 | | |
| | | | | | | 1 | | 4 | | | | 1 | 1 | 1 | | |
| | | | | | | | | 1 | 1 | | | | | | | |
| hsa-miR-505 | | | | | | | | 1 | | | | | | | | |
| | | | | | | 1 | | 3 | | 1 | | | | 2 | | |
| | | | | | | 1 | | 3 | | 1 | | | | 2 | | |
| hsa-miR-146b | | | | | | | | 3 | | | | | | 1 | | |
| | 1 | | | 1 | 1 | 1 | | 3 | | | 1 | 1 | 1 | 1 | | 1 |
| hsa-miR-29b-1 | | | | | | | | 1 | 1 | | | | 1 | | | |
| | | | 1 | | | | | 7 | 3 | 1 | | 4 | 1 | 1 | | |
| | | | | | 1 | | | 4 | | | | | | | | |
| hsa-miR-486 | | | | | | | | | | | | | | | | |
| | | | | | 1 | | | 1 | | | | 3 | 1 | 1 | | |
| | 1 | | | | | | | | | | | | | | | |
| | | | | | 4 | | | 2 | | 1 | | 1 | 1 | 3 | | |
| | | | 1 | | | 1 | | 3 | | 1 | | | | | | |
| | | | 1 | | 1 | 1 | | 4 | 2 | 1 | | 5 | 1 | 1 | | |
| | | | | | | | | 1 | | | | | | | | |
| | | | | 1 | | | | 1 | | | | 1 | | | | |
| | | | | | | | | 1 | | | | 1 | | | | |
| hsa-miR-34a | | | | | | | | 2 | | | | | | 1 | | |
| | | | | | 1 | | | 2 | 1 | 1 | | 2 | 1 | 1 | | |
| | | | | | 1 | | | 1 | | | | 1 | | | | |
| hsa-miR-141 | | | | 1 | 1 | | | 3 | | | | 1 | | 1 | | |
| | | 3 | 1 | 3 | 6 | 1 | | 11 | | 3 | | 1 | 3 | 4 | 1 | 1 |
| hsa-miR-98 | | | | | | | | | | | | | | | | |
| | | | | 1 | | | | 1 | 1 | | | 1 | | | | |
| | | | | 1 | | | | 3 | 1 | 1 | | 1 | 1 | | | |
| hsa-miR-576 | | 1 | | 1 | 1 | 1 | | 1 | | | | | 1 | 1 | | |
| | | 1 | | 1 | 2 | 1 | | 3 | | | | | 1 | 1 | | |
| | | | | | | | | 1 | | | | 1 | | | | |
| hsa-miR-9-1 | | | | | | | | | | | | | | | | |
| | | | | 1 | 1 | | | 2 | | | | 1 | 1 | 1 | | |
| | | | | 1 | | | | 1 | | | | | | 1 | | |
| | | | | 1 | 1 | | | 4 | | | | 1 | 1 | 1 | | |
| | | | | 1 | | | | 1 | | | | 1 | | 1 | | |
| | | | | 1 | 1 | | | 4 | | | | 1 | 1 | 1 | | |
| hsa-miR-142 | 3 | | | 1 | 2 | | | 4 | | 1 | | | 1 | | | |
| | 6 | 3 | | 3 | 7 | 3 | 1 | 13 | 2 | 4 | | 3 | 8 | 3 | | |
| hsa-miR-92a-1 | | | | 1 | 1 | | | 1 | | | | | 1 | 1 | | |
| | | 1 | | 5 | 2 | | | 3 | | | | | 2 | 2 | | |
| | | 1 | | 2 | 1 | | | 1 | | | | | 1 | 1 | | |
| | | 1 | | | 1 | | | 1 | | | | | | | | |
| | | 1 | | 5 | 2 | | | 2 | | | | | 2 | 2 | | |
| hsa-miR-502 | | | | | | | | 1 | | | | | | | | |
| | | | | | 2 | | | 1 | | | | | 1 | | | |
| hsa-miR-140 | | 1 | | 1 | 1 | | 1 | 1 | | | | 1 | | 1 | | 1 |
| | | 2 | | 2 | 2 | 1 | 1 | 2 | 1 | 1 | | 3 | 1 | 2 | | 1 |
| | | | | | 1 | | | 1 | 1 | 1 | | 1 | 1 | | | |
| | | | | | | | | 1 | | | | | | | | |
| | | | | | 1 | | | 1 | | | | | | | | |
| hsa-miR-100 | | 1 | | | 1 | | | 1 | | | | | | | | |
| | | 1 | | | 1 | | | 4 | | | | | | | | |
| | | | | | | | | 1 | | | | | | | | |
| | | | | | | | | 3 | | | | | | | | |
| | | | | | | | | 1 | | | | | | | | |
| | | | | | | | | 1 | | | | | | | | |
| | | | | | | | | 1 | | | | | | | | |
| | | | | | | | | 1 | | | | | | | | |
| | | | | | | | | 2 | | | | | | | | |
| | | | | | | | | 2 | | | | | | | | |
| hsa-miR-329-1 | | | | | | | | | | | | | | | | |
| | | | | | | | | | | 1 | | | | | | |
| hsa-miR-454 | | | 1 | 1 | | | | 2 | | 2 | | 1 | 1 | 2 | | |
| | | 1 | 1 | 2 | 1 | 2 | | 2 | 1 | 5 | | 2 | 3 | 2 | | 1 |
| | | | | | | | | | | 2 | | 1 | 1 | 1 | | 2 |

| SUMMARY | ARID3A | ATF3 | BCL3 | CEBPB | CREB1 | EGR1 | ETS1 | ETV6 | EZH2 |
|---|---|---|---|---|---|---|---|---|---|
| Total # of binding | 73 | 93 | 56 | 115 | 61 | 104 | 70 | 28 | 46 |
| sites in all miRNAs | | | | | | | | | |

promoter = within 2 kb
enhancer = within 10 kb

| microRNA | locus | Home Gene | Promoter/Enhance | Notes | ARID3A | ATF3 | BCL3 | CEBPB | CREB1 | EGR1 | ETS1 | ETV6 | EZH2 |
|---|---|---|---|---|---|---|---|---|---|---|---|---|---|
| hsa-miR-32 | 9q31.3 | TMEM245 | TMEM245 promoter | | 1 | 1 | | | 1 | 2 | 1 | | |
| | | | TMEM245 enhancer | | 5 | 4 | | 5 | 2 | 3 | 1 | | |
| | | | TMEM245 promoter | low H3K4me3 signal | 1 | 1 | | 1 | | | | | |
| | | | TMEM245 enhancer | | 5 | 4 | 1 | 4 | 1 | | | 2 | |
| | | | TMEM245 promoter | low H3K4me3 signal | | | | 1 | | | | | |
| | | | TMEM245 enhancer | | 1 | 1 | 1 | 2 | | | | | |
| | | | miR-32 promoter | low H3K4me3 signal | | | | | | | | | |
| | | | miR-32 enhancer | | 1 | 2 | 1 | 1 | | 1 | 1 | | |
| hsa-miR-138-1 | 3p21.3 | independent | miR-138-1 promoter | low H3K4me3 signal | | | | | | | | | |
| | | | miR-138-1 enhancer | | | 1 | 2 | | 1 | | | | |
| hsa-miR-103-1 | 5q34 | PANK3 | PANK3 promoter | | 1 | 1 | | | 1 | 1 | 1 | | |
| | | | PANK3 enhancer | | 1 | 2 | | 3 | 3 | 1 | 1 | | |
| | | | PANK3 promoter | | 1 | 1 | | | 1 | 1 | 1 | | |
| | | | PANK3 enhancer | | 1 | 2 | | 3 | 3 | 1 | 1 | | |
| | | | miR-103-1 promoter | low H3K4me3 signal | | | | | | | | | |
| | | | miR-103-1 enhancer | | | | | 1 | | | | | |
| hsa-miR-505 | Xq27.1 | ATP11C | ATP11C promoter | | | | | | | 1 | 1 | | |
| | | | ATP11C enhancer | | 1 | | | 1 | | 2 | 1 | | |
| | | | miR-505 promoter | low H3K4me3 signal | | | | 1 | | | | | |
| | | | miR-505 enhancer | | 1 | | | 1 | | 2 | 1 | | |
| hsa-miR-146b | 10q24.3 | independent | miR-146b promoter | | | 2 | | 1 | 1 | 2 | | | 1 |
| | | | miR-146b enhancer | | | 4 | 1 | 3 | 2 | 4 | 1 | | 1 |
| hsa-miR-29b-1 | 7q32.3 | LOC646329 | LOC646329 promoter | | 1 | 1 | 1 | 2 | | 1 | 1 | 1 | |
| | | | LOC646329 enhancer | | 6 | 9 | 5 | 10 | 2 | 3 | 6 | 1 | |
| | | | miR-29b-1 promoter | low H3K4me3 signal | | | | | | | | | |
| | | | miR-29b-1 enhancer | | | 4 | 4 | 4 | | 2 | 3 | | |
| hsa-miR-486 | 8p11.2 | NKX6-3 and | NKX6-3 promoter | low H3K4me3 signal | | | | | | | | | |
| | | ANK1 | | | | | | | | | | | |
| | | | NKX6-3 enhancer | | 1 | 2 | | | | 3 | 1 | | |
| | | | ANK1 promoter | | | | | | | | | | |
| | | | ANK1 enhancer | | | 1 | 1 | | 1 | | | | |
| | | | ANK1 promoter | low H3K4me3 signal | | | | | | | | | |
| | | | ANK1 enhancer | | 3 | 2 | 2 | 2 | | | 1 | 1 | 1 |
| | | | ANK1 promoter | | | 1 | | 2 | | 3 | 2 | | 1 |
| | | | ANK1 enhancer | | 1 | 4 | | 2 | | 6 | 3 | | 1 |
| | | | ANK1 promoter | | | | | | | | | | 2 |
| | | | ANK1 enhancer | | | 1 | | 1 | 2 | | | | 4 |
| | | | miR-486 promoter | low H3K4me3 signal | | | | | | | | | |
| | | | miR-486 enhancer | | | 1 | 1 | | | 1 | | | 1 |
| hsa-miR-34a | 1p36.23 | mir-34aHG | mir-34aHG Promoter | | 1 | | 1 | | | | | | 1 |
| | | | mir-34aHG Enhancer | | 2 | | 2 | 1 | | | | | 1 |
| | | | miR-34a promoter | low H3K4me3 signal | | 1 | | | | | | | |
| | | | miR-34a enhancer | | | 3 | | 3 | 1 | 1 | | | |
| hsa-miR-141 | 12p13.3 | miR-200C HG | miR-200C HG promoter | | | 1 | 1 | 1 | 1 | 2 | 2 | 1 | |
| | | | miR-141 promoter | | | | 1 | 1 | | 1 | 2 | | |
| | | | miR-141/miR-200C HG | miRNAs share | 3 | 2 | 5 | 3 | 2 | 6 | 6 | 4 | |
| | | | enhancer | regulatory elements | | | | | | | | | |
| hsa-miR-98 | Xp11.2 | HUWE1 | HUWE1 promoter | low H3K4me3 signal | | | | 1 | | | | 1 | |
| | | | HUWE1 enhancer | | | | | 1 | | | | 1 | |
| | | | HUWE1 promoter | | | | | | | | | | |
| | | | HUWE1 enhancer | | 1 | | 1 | | 1 | 3 | | | |
| | | | miR-98 promoter | low H3K4me3 signal | | | | | | | | | |
| | | | miR-98 enhancer | | | | | | | | | | |
| hsa-miR-576 | 4q25 | SEC24B | SEC24B promoter | | 2 | | | 1 | 1 | 1 | 1 | | |
| | | | SEC24B enhancer | | 3 | 2 | | 3 | 1 | 3 | 1 | | |
| | | | miR-576 promoter | low H3K4me3 signal | | | | | | | | | |
| | | | miR-576 enhancer | | | | | | | | | | |
| hsa-miR-9-1 | 1q22 | C1orf61 | C1orf61 Promoter | low H3K4me3 signal | | | | | 1 | 2 | | | 2 |
| | | | C1orf61 Enhancer | | | 2 | 1 | 1 | 3 | 3 | | 1 | 5 |
| | | | C1orf61 Promoter | | | 2 | | 1 | 1 | 1 | | | 1 |
| | | | C1orf61 Enhancer | | | 2 | 1 | 1 | 2 | 4 | | | 6 |
| | | | miR-9-1 promoter | | | 1 | | | 1 | 1 | | | 1 |
| | | | miR-9-1 enhancer | | | 2 | 1 | 1 | 2 | 4 | | | 5 |
| hsa-miR-142 | 17q22 | independent | miR-142 promoter | | 1 | 2 | 3 | | | 3 | 3 | 3 | |
| | | | miR-142 enhancer | | 4 | 5 | 9 | 6 | 3 | 9 | 11 | 11 | 3 |
| hsa-miR-92a-1 | 13q31.3 | miR-17HG/ | miR-17 promoter | | 1 | | | | | | 1 | | 1 |
| | | miR-19B1 | | | | | | | | | | | |
| | | | miR-17 HG enhancer | | 5 | 1 | 2 | 3 | 1 | 2 | 4 | | 1 |
| | | | miR-19B1 promoter | | 2 | | 1 | | | 1 | 2 | | |
| | | | miR-92a-1 promoter | | | | | | | | 1 | | |
| | | | miR-19B1/miR-92a-1 | share regulatory | 4 | | 2 | 1 | | 2 | 2 | | 1 |
| | | | | elements | | | | | | | | | |
| hsa-miR-502 | Xp11.2 | CLCN5 | CLCN5 promoter | | | | | | | | | | |
| | | | CLCN5 enhancer | | | | | 1 | | 1 | | | |
| | | | miR-502 promoter | low H3K4me3 signal | | | | | | | | | |
| | | | miR-502 promoter | | | | | | | | | | |
| hsa-miR-140 | 16q22.1 | WWP2 | WWP2 Promoter | | 1 | 1 | | | | 1 | 1 | | |
| | | | WWP2 Enhancer | | 2 | 3 | | 2 | 1 | 4 | 2 | | |
| | | | WWP2 Promoter | low H3K4me3 signal | | | | | | | | | |
| | | | WWP2 Enhancer | | 2 | | | 3 | | | | | |
| | | | WWP2 Promoter | low H3K4me3 signal | | | | | | | | | |
| | | | WWP2 Enhancer | | | | | 1 | | | | | |
| | | | WWP2 Promoter | High H3K4me3 | | | | | | | | | |
| | | | WWP2 Enhancer | | | | | 1 | | | | | |
| | | | miR-140 promoter | low H3K4me3 signal | | | | | | | | | |
| | | | miR-140 enhancer | | | | | 1 | | | 1 | | |
| hsa-miR-100 | 11q24.1 | miR-100HG | miR-100HG Promoter | | | 1 | | | 2 | | | | |
| | | | miR-100HG Enhancer | | | 4 | | 6 | 2 | | | | |
| | | | miR-100HG Promoter | | | 1 | | 2 | | | | | |
| | | | miR-100HG Enhancer | | 3 | 1 | 4 | | 1 | | | | |
| | | | miR-100HG Promoter | low H3K4me3 signal | | | | | | | | | 1 |
| | | | miR-100HG Enhancer | | | | | 1 | | 1 | | | 2 |
| | | | miR-100HG Promoter | | | 1 | | | 1 | 1 | | | |
| | | | miR-100HG Enhancer | | | 1 | | | 1 | 1 | | | 1 |
| | | | miR-100HG Promoter | low H3K4me3 signal | | | | 1 | | | | | |
| | | | miR-100HG Enhancer | | | | | 1 | | | | | |
| | | | miR-100HG Promoter | | | | | | | | | | |
| | | | miR-100HG Promoter | | | | | | | | | | |
| | | | miR-100HG Enhancer | | 1 | | | 4 | | | | | |
| | | | miR-100 promoter | | | | | 1 | | | | | |
| | | | miR-100 enhancer | | 1 | | | 3 | | | | | |
| hsa-miR-329-1 | 14q32.3 | independent | miR-329-1 promoter | low H3K4me3 signal | | | | | 2 | | | | |
| | | | miR-329-1 enhancer | low histone mark | | | | | 3 | | | | |
| | | | | signals | | | | | | | | | |
| hsa-miR-454 | 17q22 | SKA2 | SKA2 promoter | | 1 | 1 | | 1 | 1 | 2 | | | 1 |
| | | | SKA2 enhancer | | 1 | 1 | 1 | 4 | 3 | 4 | 1 | 1 | 1 |
| | | | miR-454 promoter | | | | | 1 | | | | | |
| | | | miR-454 enhancer | | | | | 1 | 2 | | 1 | | |

| | | | | SUMMARY | FOS | HDAC2 | IRF1 | JUN | JUNB | KAT2B | KDM5B |
|---|---|---|---|---|---|---|---|---|---|---|---|
| | | | | Total # of binding | 147 | 96 | 53 | 69 | 41 | 10 | 53 |
| | | | | sites in all miRNAs | | | | | | | |
| promoter = within 2 kb | | | | | | | | | | | |
| enhancer = within 10 kb | | | | | | | | | | | |
| microRNA | locus | Home Gene | Promoter/Enhance | Notes | FOS | HDAC2 | IRF1 | JUN | JUNB | KAT2B | KDM5B |
| hsa-miR-32 | 9q31.3 | TMEM245 | TMEM245 promoter | | 1 | 1 | 1 | 1 | 1 | | 1 |
| | | | TMEM245 enhancer | | 5 | 5 | 5 | 3 | 4 | | 1 |
| | | | TMEM245 promoter | low H3K4me3 signal | 1 | 1 | 1 | | | | |
| | | | TMEM245 enhancer | | 6 | 4 | 4 | 3 | 5 | | |
| | | | TMEM245 promoter | low H3K4me3 signal | 1 | | | | | | |
| | | | TMEM245 enhancer | | 2 | 1 | | | 2 | | |
| | | | miR-32 promoter | low H3K4me3 signal | | | | | | | |
| | | | miR-32 enhancer | | 4 | 1 | | 1 | | | |
| hsa-miR-138-1 | 3p21.3 | independent | miR-138-1 promoter | low H3K4me3 signal | 1 | | | | | 1 | |
| | | | miR-138-1 enhancer | | 3 | | | 1 | 1 | 1 | |
| hsa-miR-103-1 | 5q34 | PANK3 | PANK3 promoter | | 1 | | 2 | 1 | | | 1 |
| | | | PANK3 enhancer | | 3 | 1 | 2 | 2 | 2 | | 2 |
| | | | PANK3 promoter | | 1 | | 2 | 1 | | | 2 |
| | | | PANK3 enhancer | | 3 | 1 | 2 | 2 | 2 | | 2 |
| | | | miR-103-1 promoter | low H3K4me3 signal | | | | | | | |
| | | | miR-103-1 enhancer | | 2 | | | | | | |
| hsa-miR-505 | Xq27.1 | ATP11C | ATP11C promoter | | | | 1 | | | | 1 |
| | | | ATP11C enhancer | | 1 | | 1 | | | | 2 |
| | | | miR-505 promoter | low H3K4me3 signal | | | | | | | |
| | | | miR-505 enhancer | | | | 1 | | | | 2 |
| hsa-miR-146b | 10q24.3 | independent | miR-146b promoter | | 1 | 1 | 1 | | | | 1 |
| | | | miR-146b enhancer | | 3 | 3 | 2 | 1 | | | 3 |
| hsa-miR-29b-1 | 7q32.3 | LOC646329 | LOC646329 promoter | | 3 | 1 | 1 | 3 | 1 | | |
| | | | LOC646329 enhancer | | 9 | 5 | 1 | 8 | 1 | | |
| | | | miR-29b-1 promoter | low H3K4me3 signal | | | | | | | |
| | | | miR-29b-1 enhancer | | 3 | 1 | | 1 | | | |
| hsa-miR-486 | 8p11.2 | NKX6-3 and | NKX6-3 promoter | low H3K4me3 signal | | | | | | | |
| | | ANK1 | | | | | | | | | |
| | | | NKX6-3 enhancer | | 2 | 3 | 1 | 2 | 1 | | 1 |
| | | | ANK1 promoter | | | | | | | | |
| | | | ANK1 enhancer | | 2 | | | 1 | | | |
| | | | ANK1 promoter | low H3K4me3 signal | | | | | | | |
| | | | ANK1 enhancer | | 2 | | | 1 | 1 | | |
| | | | ANK1 promoter | | 2 | 2 | 1 | 2 | 1 | | 2 |
| | | | ANK1 enhancer | | 5 | 7 | 2 | 5 | 2 | | 3 |
| | | | ANK1 promoter | | | | | | | | |
| | | | ANK1 enhancer | | | 2 | | | | | |
| | | | miR-486 promoter | low H3K4me3 signal | | | | | | | |
| | | | miR-486 enhancer | | 2 | | | 1 | | | |
| hsa-miR-34a | 1p36.23 | mir-34aHG | mir-34aHG Promoter | | | | | | | | |
| | | | mir-34aHG Enhancer | | 1 | 1 | | | | 1 | |
| | | | miR-34a promoter | low H3K4me3 signal | | | | | | | |
| | | | miR-34a enhancer | | 1 | | | 1 | 1 | | |
| hsa-miR-141 | 12p13.3 | miR-200C HG | miR-200C HG promoter | | 2 | 2 | | 1 | 1 | | 1 |
| | | | miR-141 promoter | | 1 | 1 | | 1 | | | 1 |
| | | | miR-141/miR-200C HG | miRNAs share | 3 | 6 | 1 | 3 | 3 | | 2 |
| | | | enhancer | regulatory elements | | | | | | | |
| hsa-miR-98 | Xp11.2 | HUWE1 | HUWE1 promoter | low H3K4me3 signal | 1 | | | | | | |
| | | | HUWE1 enhancer | | 2 | | | | 2 | | |
| | | | HUWE1 promoter | | | | | | | | |
| | | | HUWE1 enhancer | | 1 | | 1 | 1 | | | |
| | | | miR-98 promoter | low H3K4me3 signal | | | | | | | |
| | | | miR-98 enhancer | | 1 | | | | | | |
| hsa-miR-576 | 4q25 | SEC24B | SEC24B promoter | | 1 | 1 | 1 | 1 | | 1 | 1 |
| | | | SEC24B enhancer | | 6 | 1 | 1 | 1 | 1 | 1 | 1 |
| | | | miR-576 promoter | low H3K4me3 signal | | | | | | | |
| | | | miR-576 enhancer | | | | | | | | |
| hsa-miR-9-1 | 1q22 | C1orf61 | C1orf61 Promoter | low H3K4me3 signal | | | | | | | |
| | | | C1orf61 Enhancer | | 1 | 1 | 1 | 2 | | | 2 |
| | | | C1orf61 Promoter | | 1 | 1 | 1 | 1 | | | 1 |
| | | | C1orf61 Enhancer | | 1 | 2 | 1 | 2 | | | 2 |
| | | | miR-9-1 promoter | | | | 1 | 2 | | | 2 |
| | | | miR-9-1 enhancer | | 1 | 2 | 1 | 2 | | | 2 |
| hsa-miR-142 | 17q22 | independent | miR-142 promoter | | | 2 | 2 | 2 | 1 | | 2 |
| | | | miR-142 enhancer | | 2 | 8 | 7 | 6 | 4 | | 6 |
| hsa-miR-92a-1 | 13q31.3 | miR-17HG/ | miR-17 promoter | | | 1 | | | 1 | | |
| | | miR-19B1 | | | | | | | | | |
| | | | miR-17 HG enhancer | | 1 | 3 | | | 1 | | |
| | | | miR-19B1 promoter | | | 1 | | | | | |
| | | | miR-92a-1 promoter | | | 1 | | | | | |
| | | | miR-19B1/miR-92a-1 | share regulatory | 1 | 2 | | | 1 | | |
| | | | | elements | | | | | | | |
| hsa-miR-502 | Xp11.2 | CLCN5 | CLCN5 promoter | | | 2 | | | | | |
| | | | CLCN5 enhancer | | | 3 | | | | | |
| | | | miR-502 promoter | low H3K4me3 signal | 1 | | | | | | |
| | | | miR-502 promoter | | 1 | | | | | | |
| hsa-miR-140 | 16q22.1 | WWP2 | WWP2 Promoter | | 1 | 1 | | | | | 1 |
| | | | WWP2 Enhancer | | 1 | 4 | 1 | 1 | | | 2 |
| | | | WWP2 Promoter | low H3K4me3 signal | | | | | | | |
| | | | WWP2 Enhancer | | 2 | 4 | | | | | |
| | | | WWP2 Promoter | low H3K4me3 signal | | | | | | | |
| | | | WWP2 Enhancer | | 1 | | | | | | |
| | | | WWP2 Promoter | High H3K4me3 | | | | | | | |
| | | | WWP2 Enhancer | | 1 | | | | | | |
| | | | miR-140 promoter | low H3K4me3 signal | | | | | | | |
| | | | miR-140 enhancer | | | | | | | | |
| hsa-miR-100 | 11q24.1 | miR-100HG | miR-100HG Promoter | | 1 | | | | | | |
| | | | miR-100HG Enhancer | | 8 | | | | 1 | | |
| | | | miR-100HG Promoter | | 3 | | | | | | |
| | | | miR-100HG Enhancer | | 9 | | | | | | |
| | | | miR-100HG Promoter | low H3K4me3 signal | | | | | | | |
| | | | miR-100HG Enhancer | | | | | | | | |
| | | | miR-100HG Promoter | | 1 | | | | | | |
| | | | miR-100HG Enhancer | | 2 | | | | | | |
| | | | miR-100HG Promoter | low H3K4me3 signal | 1 | | | | | | |
| | | | miR-100HG Enhancer | | 3 | | | | | | |
| | | | miR-100HG Promoter | | 1 | | | | | | |
| | | | miR-100HG Promoter | | 1 | | | | | | |
| | | | miR-100HG Enhancer | | 5 | 1 | | | | | |
| | | | miR-100 promoter | | | | | | | | |
| | | | miR-100 enhancer | | 3 | 1 | | | | | |
| hsa-miR-329-1 | 14q32.3 | independent | miR-329-1 promoter | low H3K4me3 signal | | | | | | | |
| | | | miR-329-1 enhancer | low histone mark | | | | | | | |
| | | | | signals | | | | | | | |
| hsa-miR-454 | 17q22 | SKA2 | SKA2 promoter | | 1 | 1 | 1 | 1 | | | 1 |
| | | | SKA2 enhancer | | 3 | 3 | 2 | 1 | | 1 | 2 |
| | | | miR-454 promoter | | | | | | | | |
| | | | miR-454 enhancer | | | | | | | | |

| | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
|---|---|---|---|---|---|---|---|---|---|---|---|---|---|---|---|---|
| | 30 | 127 | 41 | 2 | 10 | 66 | 6 | 2 | 49 | 52 | 42 | 35 | 12 | 4 | 81 | 23 |
| microRNA | MBD2 | MYC | NFE2L2 | NFYA | PHF21A | RBBP5 | RBM14 | RBM15 | RBM25 | RBM39 | RELB | RFX5 | RLF | RUNX1 | RXRA | SIX5 |
| hsa-miR-32 | 1 | 2 | | | | 2 | | | 1 | 1 | 1 | 1 | | | 1 | 1 |
| | 1 | 5 | 1 | | | 2 | | | 2 | 1 | 1 | 3 | | | 4 | 1 |
| | | 3 | 1 | | | | | | 1 | | 1 | 2 | | | 4 | |
| | | | | | | | | | | | 1 | | | | 1 | |
| | | | | | | | | | 1 | | | | | | 2 | |
| hsa-miR-138-1 | | 1 | | | | | | | | | | | | | | |
| | | 2 | | | | | | | | | | | | | | |
| hsa-miR-103-1 | 1 | 1 | 1 | | | | | | | 1 | 1 | 1 | | | 1 | 1 |
| | 1 | 2 | 1 | | 1 | 1 | | | 1 | 1 | 3 | 1 | 1 | | 2 | 1 |
| | 1 | 1 | 1 | | 1 | 1 | | | | 1 | 1 | 1 | 1 | | 1 | 1 |
| | 1 | 2 | 1 | | 1 | 1 | | | 1 | 1 | 3 | 1 | 1 | | 2 | 1 |
| hsa-miR-505 | 1 | 1 | | | | 1 | | | | 1 | | | | | 1 | 1 |
| | 1 | 2 | | | | 1 | | | 2 | 1 | | | | | 1 | |
| | 1 | 2 | | | | 1 | | | 2 | 1 | | | | | 1 | 1 |
| hsa-miR-146b | 1 | | | | | 1 | | | 1 | 1 | | | | | 2 | |
| | 2 | 2 | | | 1 | 2 | | | 1 | 2 | 1 | | 1 | | 3 | 1 |
| hsa-miR-29b-1 | | 1 | 1 | | | 1 | | | | 1 | 1 | 1 | | | 1 | |
| | 1 | 5 | 5 | | | 3 | | | 3 | 2 | 1 | 1 | | | 9 | 3 |
| | | 3 | 2 | | | | | | | | | | | | 2 | 1 |
| hsa-miR-486 | | | | | | | | | | | | | | | | |
| | | 1 | 1 | | | 2 | | | 4 | | 1 | | | | | |
| | | 1 | | | | | | | 1 | | 3 | | | | | |
| | 2 | 3 | 1 | | 1 | 1 | | | | | | 1 | 1 | 1 | | |
| | 2 | 5 | 2 | | 1 | 3 | | | 4 | | 1 | 1 | 1 | 1 | | |
| | | | | | | 1 | | | | | | | | | | |
| | | 1 | | | | 2 | | | 2 | | | | | | 1 | |
| | | | | | | | | | 1 | | | | | | | |
| hsa-miR-34a | | 1 | | | | | | | | 1 | | | | | | |
| | | 1 | | | | | | | | 2 | 1 | | 1 | | 2 | |
| | | 1 | | | | | | | | | | 1 | | | | |
| hsa-miR-141 | | 3 | 2 | | | 1 | | | | 1 | 1 | | | | 1 | |
| | 1 | 5 | 3 | | | 3 | | | | 2 | 4 | 1 | 1 | | 7 | 1 |
| hsa-miR-98 | | | | | | | | | | | | | | | | |
| | | | | | | | | | | | | 1 | | | | |
| | | | | | | 1 | | | 1 | 1 | | | | | | |
| | 1 | 2 | 2 | | | 2 | | | 3 | 1 | 1 | | 1 | | 1 | |
| hsa-miR-576 | | 1 | | | | 1 | | | 1 | 1 | | | | | 1 | |
| | | 3 | | | | 1 | | | 1 | 1 | 1 | | | | | 2 |
| hsa-miR-9-1 | | | | | | | | | | | | | | | | |
| | | 1 | | | | 3 | | | 1 | 1 | 1 | 1 | | | | |
| | | 1 | | | | 1 | | | 1 | | | 1 | | | | |
| | | 1 | | | | 2 | | | 1 | | 1 | 1 | | | | |
| | | 1 | | | | 2 | | | 1 | | 1 | 1 | | | | |
| | | 1 | | | | 2 | | | 1 | | 1 | 1 | | | | |
| hsa-miR-142 | 1 | 2 | | | | 2 | 2 | 1 | | 1 | | 1 | | | | |
| | 7 | 12 | | | 2 | 4 | 4 | 1 | 1 | 8 | 6 | 5 | 3 | 2 | 3 | 4 |
| hsa-miR-92a-1 | | 1 | | | | 1 | | | | 1 | | 1 | | | | |
| | | 3 | | | | 2 | | | | 2 | | 1 | | | 3 | |
| | | 2 | | | | 1 | | | | 1 | | | | | 1 | |
| | | 1 | | | | | | | | | | | | | | |
| | | 3 | | | | 2 | | | | 2 | | 1 | | | 1 | |
| hsa-miR-502 | | 2 | | | | 1 | | | | | | | | | | |
| | | 2 | | | | 1 | | | | | | | | | | |
| hsa-miR-140 | | 1 | 1 | | | 1 | | | 1 | 1 | | | | | 1 | 1 |
| | 1 | 2 | 3 | | | 2 | | | 3 | 2 | 1 | 1 | | | 2 | 2 |
| | | | | | | | | | | | | | | | 3 | |
| | | 1 | 1 | | | | | | | 1 | | | | | 1 | |
| | | 1 | 1 | | | | | | | 2 | | | | | 1 | |
| | | | | | | | | | | 2 | | | | | 2 | |
| hsa-miR-100 | | | | | | | | | | | | | | | 1 | |
| | | 3 | 2 | | | | | | | | | | | | 4 | |
| | | | 1 | | | | | | | | | | | | | |
| | | 1 | 3 | | | | | | | | | 1 | | | 1 | |
| | | | 1 | | | | | | | | | | | | | |
| | | 1 | | | | | | | | | | | | | 1 | |
| | | 2 | | | | | | | | | | | | | 1 | |
| | | 1 | | | | | | | | | | | | | | |
| | | 2 | | | | | | | | | | | | | 1 | |
| | | 1 | | | | | | | | | | | | | | |
| | | 1 | | | | | | | | | | | | | | |
| | | 3 | | | | | | | | | | | | | | |
| | | 2 | | | | | | | | | | | | | | |
| hsa-miR-329-1 | | | | | | | | | | | | | | | | |
| | | | | | | | | | 1 | | | | | | | |
| hsa-miR-454 | 1 | 1 | | 1 | 1 | 1 | | | 1 | 1 | | 1 | | | 1 | |
| | 1 | 4 | 1 | 1 | 1 | 2 | | | 2 | 1 | 2 | 1 | | | 1 | |
| | | 1 | 1 | | | | | | 1 | | | | | | 1 | |

| | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
|---|---|---|---|---|---|---|---|---|---|---|---|---|---|---|---|---|
| | 11 | 21 | 7 | 39 | 53 | 18 | 3 | 164 | 16 | 30 | 3 | 49 | 45 | 51 | 1 | 7 |
| microRNA | SNRNP70 | STAT1 | STAT2 | TAF7 | TRIM22 | U2AF1 | U2AF2 | YY1 | ZFP91 | ZKSCAN1 | ZMIZ1 | ZMYM3 | ZNF207 | ZNF217 | ZNF280A | ZNF574 |
| hsa-miR-32 | | 1 | | | 1 | | | 1 | | 1 | 1 | 2 | 1 | 1 | | |
| | | 1 | 1 | | 1 | 1 | | 6 | | 1 | 1 | 3 | 1 | 1 | | |
| | | 1 | | | | | | 6 | | | | | | 1 | | |
| | | | | | | | | 1 | | | | | | 1 | | |
| | | | | | | | | 1 | 1 | | | | | | | |
| hsa-miR-138-1 | | | | | | | | 1 | | | | | | | | |
| | | | | | | | | 1 | | | | | | | | |
| hsa-miR-103-1 | | | | | | | | 1 | | | | | 1 | 1 | | |
| | | | | | | 1 | | 4 | | | | 1 | 1 | 1 | | |
| | | | | | | 1 | | 2 | | | | | 1 | 1 | | |
| | | | | | | 1 | | 4 | | | | 1 | 1 | 1 | | |
| | | | | | | | | 1 | 1 | | | | | | | |
| hsa-miR-505 | | | | | | | | 1 | | | | | | | | |
| | | | | | | 1 | | 3 | | 1 | | | | 2 | | |
| | | | | | | 1 | | 3 | | 1 | | | | 2 | | |
| hsa-miR-146b | | | | | | | | 3 | | | | | | 1 | | |
| | 1 | | | 1 | 1 | 1 | | 3 | | | 1 | 1 | 1 | 1 | | 1 |
| hsa-miR-29b-1 | | | | | | | | 1 | 1 | | | | 1 | | | |
| | | | 1 | | | | | 7 | 3 | 1 | | 4 | 1 | 1 | | |
| | | | | | 1 | | | 4 | | | | | | | | |
| hsa-miR-486 | | | | | | | | | | | | | | | | |
| | | | | | 1 | | | 1 | | | | 3 | 1 | 1 | | |
| | 1 | | | | | | | | | | | | | | | |
| | | | | | 4 | | | 2 | | 1 | | 1 | 1 | 3 | | |
| | | | 1 | | | 1 | | 3 | | 1 | | | | | | |
| | | | 1 | | 1 | 1 | | 4 | 2 | 1 | | 5 | 1 | 1 | | |
| | | | | | | | | 1 | | | | | | | | |
| | | | | 1 | | | | 1 | | | | 1 | | | | |
| | | | | | | | | 1 | | | | 1 | | | | |
| hsa-miR-34a | | | | | | | | 2 | | | | | | 1 | | |
| | | | | | 1 | | | 2 | 1 | 1 | | 2 | 1 | 1 | | |
| | | | | | 1 | | | 1 | | | | 1 | | | | |
| hsa-miR-141 | | | | 1 | 1 | | | 3 | | | | 1 | | 1 | | |
| | | 3 | 1 | 3 | 6 | 1 | | 11 | | 3 | | 1 | 3 | 4 | 1 | 1 |
| hsa-miR-98 | | | | | | | | | | | | | | | | |
| | | | | 1 | | | | 1 | 1 | | | 1 | | | | |
| | | | | 1 | | | | 3 | 1 | 1 | | 1 | 1 | | | |
| hsa-miR-576 | | 1 | | 1 | 1 | 1 | | 1 | | | | | 1 | 1 | | |
| | | 1 | | 1 | 2 | 1 | | 3 | | | | | 1 | 1 | | |
| | | | | | | | | 1 | | | | 1 | | | | |
| hsa-miR-9-1 | | | | | | | | | | | | | | | | |
| | | | | 1 | 1 | | | 2 | | | | 1 | 1 | 1 | | |
| | | | | 1 | | | | 1 | | | | | | 1 | | |
| | | | | 1 | 1 | | | 4 | | | | 1 | 1 | 1 | | |
| | | | | 1 | | | | 1 | | | | 1 | | 1 | | |
| | | | | 1 | 1 | | | 4 | | | | 1 | 1 | 1 | | |
| hsa-miR-142 | 3 | | | 1 | 2 | | | 4 | | 1 | | | 1 | | | |
| | 6 | 3 | | 3 | 7 | 3 | 1 | 13 | 2 | 4 | | 3 | 8 | 3 | | |
| hsa-miR-92a-1 | | | | 1 | 1 | | | 1 | | | | | 1 | 1 | | |
| | | 1 | | 5 | 2 | | | 3 | | | | | 2 | 2 | | |
| | | 1 | | 2 | 1 | | | 1 | | | | | 1 | 1 | | |
| | | 1 | | | 1 | | | 1 | | | | | | | | |
| | | 1 | | 5 | 2 | | | 2 | | | | | 2 | 2 | | |
| hsa-miR-502 | | | | | | | | 1 | | | | | | | | |
| | | | | | 2 | | | 1 | | | | | 1 | | | |
| hsa-miR-140 | | 1 | | 1 | 1 | | 1 | 1 | | | | 1 | | 1 | | 1 |
| | | 2 | | 2 | 2 | 1 | 1 | 2 | 1 | 1 | | 3 | 1 | 2 | | 1 |
| | | | | | 1 | | | 1 | 1 | 1 | | 1 | 1 | | | |
| | | | | | | | | 1 | | | | | | | | |
| | | | | | 1 | | | 1 | | | | | | | | |
| hsa-miR-100 | | 1 | | | 1 | | | 1 | | | | | | | | |
| | | 1 | | | 1 | | | 4 | | | | | | | | |
| | | | | | | | | 1 | | | | | | | | |
| | | | | | | | | 3 | | | | | | | | |
| | | | | | | | | 1 | | | | | | | | |
| | | | | | | | | 1 | | | | | | | | |
| | | | | | | | | 1 | | | | | | | | |
| | | | | | | | | 1 | | | | | | | | |
| | | | | | | | | 2 | | | | | | | | |
| | | | | | | | | 2 | | | | | | | | |
| hsa-miR-329-1 | | | | | | | | | | | | | | | | |
| hsa-miR-454 | | | 1 | 1 | | | | 2 | | 2 | | 1 | 1 | 2 | | |
| | | 1 | 1 | 2 | 1 | 2 | | 2 | 1 | 5 | | 2 | 3 | 2 | | 1 |
| | | | | | | | | | | 2 | | 1 | 1 | 1 | | 2 |

1. A method for detecting or monitoring FSHD in a subject comprising:
detecting at least one of miR-100, miR-29b, miR-34a, miR-505 or miR-576;
detecting at least one of S100A8, F13A1, IGF1, PFN1, FBLN1, CFL1, TMSB4X, TPM4, EFEMP1, KRT16, SPP2, PROC, or PRG4; or detecting at least one other biomarker for FSHD present in plasma or another biofluid or liquid biopsy sample of the subject;
comparing quantity of the at least one biomarker in the subject to a control value of the quantity of the biomarker in an age and gender matched subject who does not have FSHD, or to the quantity of the biomarker in a serially collected sample from the same subject at a different point in time;
selecting a subject with FSHD when at least one of said biomarkers, is elevated or depressed compared to the control value for said at least one biomarker,
and, optionally,
treating the subject for FSHD.

2. The method of claim 1, wherein the biofluid or liquid biopsy sample is blood, plasma, serum, urine, stool, saliva, or combinations thereof; and/or wherein the biomarker is RNA, peptide, protein, or combinations thereof, and monitoring disease progression of FSHD, monitoring treatment response of FSHD, or predicting FSHD prognosis based on elevation or depression of the at least one biomarker.

3. The method of claim 1, wherein the biofluid or liquid biopsy sample comprises urine, sweat, tears, breast milk, bile, interstitial fluid, cytosol, peritoneal fluid, pleural fluid, amniotic fluid, semen, synovial (joint) fluid, CSF (cerebrospinal fluid), lymph, mucous, saliva, or other bodily fluids, stool or fecal matter, epithelium, hair follicles, mucosal cells or secretions (such as from bronchial, nasal, buccal, or cheek swabs), or biopsy, such as a muscle biopsy.

4. The method of claim 1, wherein the method comprises a step of selecting a subject having, or being at risk of having FSHD, for further testing or treatment when the quantity of said biomarker is significantly elevated or decreased compared to the control value.

5. The method of claim 1, wherein the at least one biomarker is elevated or depressed in a subject having FSHD.

6. The method of claim 1 for detecting or monitoring FSHD, wherein the biomarker is one or more miRNA biomarkers selected from the group consisting of miR-9, miR-29b, miR-32, miR-34a, miR-92a, miR-98, miR-100, miR-103, miR-138, miR-140-3p, miR-141, miR-142-3p, miR-146b, miR-329, miR-454, miR-486, miR-502-3p, miR-505 and miR-576.

7. The method of claim 1 for detecting or monitoring FSHD, wherein the biomarker is selected from the group consisting of at least one of miR-100, miR-29b, miR-34a, miR-505 and miR-576.

8. The method for detecting or monitoring mild FSHD of claim 1,
wherein the biomarker is at least one miRNA selected from the group consisting miR-92a, miR-138, and miR-486, wherein the subject is selected as having or as at risk of having FSHD when one or more of these miRNAs is decreased or downregulated compared to a control value; and/or
wherein the biomarker is at least one miRNA selected from the group consisting of miR-9, miR-29b, miR-32, miR-142-3p, miR-146b, miR-505 and miR-576, wherein the subject is selected as having or as at risk of having FSHD when one or more of these miRNAs is increased or upregulated compared to a control value.

9. The method for detecting or monitoring severe FSHD of claim 1,
wherein the biomarker is one or more miRNA biomarkers selected from the group consisting of miR-140-3p and miR-502-3p, wherein the subject is selected as having or as at risk of having FSHD when a level of one or more of these miRNAs is decreased or its expression is down-regulated compared to a control value; and/or
wherein the biomarker is at least one miRNA selected from the group consisting wherein the biomarker is one or more miRNA biomarkers selected from the group consisting of miR-29b, miR-32, miR-34a, miR-98, miR-100, miR-103, miR-141, miR-329, miR-454, and miR-505, wherein the subject is selected as having or as at risk of having FSHD when one or more of these miRNAs is increased or its expression is up-regulated compared to a control value.

10. The method for detecting or monitoring FSHD of claim 1, wherein the biomarker comprises miR-100.

11. The method for detecting or monitoring FSHD of claim 1, wherein the at least one biomarker comprises miR95, miR886-3p, and/or miR-502-3p, wherein when quantities of miR-95 and/or miR886-3p are increased in comparison to a control value from a subject having mild FSHD, then the subject is selected as having severe FSHD, and wherein when a quantity of miR-502-3p is decreased in comparison to a control value from a normal subject or from a subject having mild FSHD, then the subject is selected as having severe FSHD.

12. The method for detecting or monitoring FSHD of claim 1, wherein the biomarker comprises one or more protein biomarkers selected from the group consisting of S100A8, F13A1, IGF1, PFN1, FBLN1, CFL1, TMSB4X, TPM4, EFEMP1, KRT16, SPP2, PROC, and PRG4.

13. The method for detecting or monitoring FSHD of claim 1, wherein the biomarker comprises one or more protein biomarkers selected from the group consisting of S100A8, F13A1, IGF1, PFN1, FBLN1, CFL1, TMSB4X, TPM4, EFEMP1, KRT16, and SPP2, S100A8 and wherein said comparing comprises detecting an increase in expression of said protein biomarker(s) in a subject having or at risk of developing FSHD; and/or wherein the biomarker is one or more protein biomarkers selected from the group consisting of PROC and PRG4 and wherein said comparing comprises detecting an decrease in a level of or in expression of said protein biomarker(s) in a subject having or at risk of developing FSHD.

14. The method for detecting or monitoring FSHD of claim 1, wherein the biomarker comprises at least one selected from the group consisting of S100A8, IGF1, PRG4, PFN1, and TPM4 and wherein said comparing comprises detecting an increase in a level of or in expression of said at least one biomarker(s) in a subject having or at risk of developing FSHD.

15. The method for detecting or monitoring FSHD of claim 1, wherein the biomarker comprises S100A8 and wherein said comparing comprises detecting an increase in a level of or in expression of S100A8 protein and/or calprotectin in a subject having or at risk of developing FSHD.

16. The method of claim 1 for detecting or monitoring FSHD, wherein two or more biomarkers are detected which are selected from the groups consisting of miR-9, miR-29b, miR-32, miR-34a, miR-92a, miR-98, miR-100, miR-103, miR-138, miR-140-3p, miR-141, miR-142-3p, miR-146b, miR-329, miR-454, miR-486, miR-502-3p, miR-505 and miR-576, and S100A8, F13A1, IGF1, PFN1, FBLN1, CFL1, TMSB4X, TPM4, EFEMP1, KRT16, SPP2, PROC, and PRG4.

17. The method of claim 1 for detecting or monitoring FSHD, wherein at least two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, or thirty two, biomarkers are detected.

18. The method of claim 1, further comprising administering to the selected subject an antisense oligonucleotide including those described by U.S. Ser. No. 16/649,122 or EU 18859092.1.

19. The method of claim 1, further comprising administering at least one miRNA selected from the group consisting of miR-9, miR-29b, miR-32, miR-34a, miR-92a, miR-98, miR-100, miR-103, miR-138, miR-140-3p, miR-141, miR-142-3p, miR-146b, miR-329, miR-454, miR-486, miR-502-3p, miR-505 and miR-576; or at least one inhibitor of said miRNAs such as oligonucleotides that hybridize to mature miRNAs and inhibit their function including RNA oligonucleotides comprising 2′-O-methyl residues that confer increased binding affinity to RNA targets and resistance to endonuclease degradation or ZEN (naphthyl-azo) modifications that block exonuclease degradation.

20. The method of claim 1, further comprising administering to the selected subject an agent that enhances epigenetic repression of D4Z4, targets DUX4 mRNA, blocks activity of the DUX4 protein or inhibits DUX4-induced processes leading to pathology.

21. The method of claim 1, further comprising administering to the selected subject Losmapimod or other selective inhibitor of p38α/β mitogen-activated protein kinases, antisense oligonucleotides that reduce DUX4 expression, or gene therapy, such as administration of miRNAs directed against DUX4.

22. The method of claim 1, further comprising administering to the selected subject an inhibitor of hyaluronic acid biosynthesis such as 4-methylumbellifoerone, a BET inhibitor, a casein kinase 1 inhibitor and/or vitamin C, vitamin E, acetylcysteine, zinc gluconate, selenomethionine or other antioxidants.

23. The method of claim 1, further comprising treating the selected subject for FSHD with surgical correction of facial weakness, scapular bracing, scapular fusion, scapuloplexy, tendon transfer such as pectoralis major transfer or Eden-Lange procedure or for the foot the Bridle procedure, correction of foot drop, ankle-foot orthoses, physiotherapy, occupational therapy, an assistive device, aerobic exercise, strength training, or cognitive behavioral therapy (CBT).

24. The method of claim 1, further comprising conducting an eye exam to identify retinal abnormalities, a hearing test to identify hearing loss, or pulmonary function testing to establish a baseline pulmonary function or changes from a prior established baseline.

25. A method for diagnosing a subject as having FSHD comprising obtaining a biological sample from the subject, and detecting a quantity of one or more miRNA and/or one or more protein biomarkers for FSHD in the sample, and
diagnosing the subject as having FSHD when a quantity of said one or more miRNA and/or protein biomarkers is altered compared to the quantity of said one or more biomarkers in a control subject.

26. The method of claim 25, wherein the biomarkers are selected from the group consisting of miR-138, miR-486, miR-9, miR-32, miR-146b, miR-92a, miR-576, miR-142-3p, miR-505, miR-29b, miR-502-3p, miR-103, miR-98, miR-141, miR-34a, miR-140-3p, miR-100, miR-329, miR-454, miR-95, and miR-886-3p.

27. The method of claim 25, wherein the biomarkers are selected from the group consisting of S100A8, F13A1, IGF1, PFN1, FBLN1, CFL1, TMSB4X, TPM4, EFEMP1, KRT16, SPP2, PROC, and PRG4.

28. The method of claim 25, wherein both miRNA biomarkers and protein biomarkers are detected.

29. The method of claim 25, wherein the biological sample comprises blood, plasma or serum, urine, stool, saliva, or combinations thereof.

30. The method of claim 25, wherein the biological sample comprises urine, sweat, tears, breast milk, bile, interstitial fluid, cytosol, peritoneal fluid, pleural fluid, amniotic fluid, semen, synovial (joint) fluid, CSF (cerebrospinal fluid), lymph, mucous, saliva, or other bodily fluids, stool or fecal matter, epithelium, hair follicles, mucosal cells or secretions (such as from bronchial, nasal, buccal, or cheek swabs), or biopsy, such as a muscle biopsy.

31. The method of claim 25 comprising:
comparing quantity of the at least one biomarker in the subject to a control value, to the quantity in an age and gender matched subject who does not have FSHD; to the quantity in a serially collected sample from the same subject at a different point in time; or to an other suitable control,
selecting a subject having, or at risk of having FSHD, when the quantity of said biomarker is significantly elevated or decreased compared to the control value;
and, optionally,
treating the subject for FSHD.

32. A method for diagnosing a subject as having FSHD comprising obtaining a biological sample from the subject, and detecting a quantity of one or more miRNA and/or one or more protein biomarkers for FSHD in the sample, and
diagnosing the subject as having FSHD when a quantity of said one or more miRNA and/or protein biomarkers is altered compared to the quantity of said one or more biomarkers in a control subject.

33. The method of claim 32, wherein the biomarkers are selected from the group consisting of miR-138, miR-486, miR-9, miR-32, miR-146b, miR-92a, miR-576, miR-142-3p, miR-505, miR-29b, miR-502-3p, miR-103, miR-98, miR-141, miR-34a, miR-140-3p, miR-100, miR-329, miR-454, miR-95, and miR-886-3p.

34. The method of claim 32, wherein the biomarkers are selected from the group consisting of S100A8, F13A1, IGF1, PFN1, FBLN1, CFL1, TMSB4X, TPM4, EFEMP1, KRT16, SPP2, PROC, and PRG4.

35. The method of claim 32, wherein both miRNA biomarkers and protein biomarkers are detected.

36. The method of claim 32, wherein the biological sample comprises blood, plasma, or serum.

37. The method of claim 32, wherein the biological sample comprises urine, sweat, tears, breast milk, bile, interstitial fluid, cytosol, peritoneal fluid, pleural fluid, amniotic fluid, semen, synovial (joint) fluid, CSF (cerebrospinal fluid), lymph, mucous, saliva, or other bodily fluids, stool or fecal matter, epithelium, hair follicles, mucosal cells or secretions (such as from bronchial, nasal, buccal, or cheek swabs), or biopsy, such as a muscle biopsy.

38. A kit for diagnosing or monitoring FSHD comprising reagents suitable for detection of the specific miRNAs disclosed herein and/or with reagents suitable for detecting the biomarker proteins disclosed herein.

39. The kit of claim 38, wherein the reagents suitable for detection of the specific miRNAs disclosed herein are oligonucleotides complementary to said miRNAs.

40. The kit of claim 38, wherein the reagents suitable for detection of the specific miRNAs disclosed herein are oligonucleotides complementary to said miRNAs which are selected from the group consisting of one or more of miR-138, miR-486, miR-9, miR-32, miR-146b, miR-92a, miR-576, miR-142-3p, miR-505, miR-29b, miR-502-3p, miR-103, miR-98, miR-141, miR-34a, miR-140-3p, miR-100, miR-329, miR-454, miR-95, and miR-886-3p.

41. The kit of claim 38, wherein the reagents suitable for detection of the specific miRNAs disclosed herein are oligonucleotides complementary to said miRNAs which are selected from the group consisting of one or more of miR95, miR886-3p, and/or miR-502-3p.

42. The kit of claim 38, wherein the reagents suitable for detecting the biomarker proteins disclosed herein are antibodies that bind to the biomarker proteins.

43. The kit of claim 38, wherein the reagents suitable for detecting the biomarker proteins disclosed herein are antibodies that bind to the biomarker proteins which are at least one selected from the group consisting of S100A8, F13A1, IGF1, PFN1, FBLN1, CFL1, TMSB4X, TPM4, EFEMP1, KRT16, SPP2, PROC, and PRG4.
